(19) United States
(12) Patent Application Publication
(10) Pub. No.: US 2019/0154692 A1
(43) Pub. Date: May 23, 2019

(54) COMPOSITIONS AND METHODS FOR SCREENING, MONITORING AND TREATING GASTROINTESTINAL DISEASES

(71) Applicant: BioCrypton Inc., South San Francisco, CA (US)

(72) Inventors: Valeria S. Ossovskaya, San Francisco, CA (US); Olga Potapova, San Francisco, CA (US); Ilya Mazo, Rockville, MD (US)

(21) Appl. No.: 16/092,798

(22) PCT Filed: Apr. 12, 2017
(86) PCT No.: PCT/US2017/027196

§ 371 Date: Oct. 11, 2018

Related U.S. Application Data
(60) Provisional application No. 62/321,294, filed on Apr. 12, 2016.

(51) Int. Cl. G01N 33/574; G01N 33/543

(52) U.S. Cl. CPC G01N 33/57446; G01N 33/54306; G01N 33/57438; G01N 33/57419

(57) ABSTRACT

Provided are compositions and methods for screening, monitoring and treating a gastrointestinal (GI) cancer patient. In the methods, the patient's sample is screened for a profile of abnormal pathways and glycosylated protein biomarkers indicative of a GI cancer.

### CROSS-REFERENCE TO RELATED APPLICATIONS

This application claims a benefit of priority to U.S. patent application Ser. No. 62/321,294, filed Apr. 12, 2016, the entire disclosure of which is incorporated herein by reference.

### TECHNICAL FIELD

This invention relates to compositions and methods for screening, diagnosing, monitoring and treating gastrointestinal (GI) diseases, including colorectal cancer, gastric cancer, liver cancer, and pancreatic cancer.

### BACKGROUND OF THE INVENTION

Gastrointestinal (GI) diseases are complex chronic human disorders. GI diseases include colorectal cancer, gastric cancer, liver cancer, and pancreatic cancer. GI cancers account for a large percentage of cancer mortalities.

Colorectal cancer (CRC) is the third most commonly diagnosed cancer and the third leading cause of cancer death in both men and women in the United States. Diet, environmental, genetic and inflammation factors contribute in the CRC etiology. Colorectal cancer usually develops over a period of 10 to 20 years. A significant progress has been made in the past decade in reducing the CRC incidence and death rates in the United States, largely due to prevention and early detection of colorectal cancer.

About 25,000 new stomach (gastric) cancer cases are reported in the United States annually. Before a stomach cancer develops, pre-cancerous changes often occur in the inner lining, mucosa, of the stomach. These early changes rarely cause symptoms and therefore often go undetected. The overall 5-year survival rate for patients with stomach cancer is 29% as most patients with stomach cancer are diagnosed after the cancer has already spread to other parts of the body. If stomach cancer is diagnosed and treated before it has spread outside the stomach, the 5-year survival rate is 65%. This data supports a high unmet need for developing a molecular test for detecting stomach cancer at early stages while the patient has not developed symptoms and the cancer has not spread outside the stomach.

Liver cancer is the 10th most common cancer and the 5th most common cause of cancer death among men. It is also the 8th most common cause of cancer death among women. The overall 5-year survival rate for patients with liver cancer is 18%. For 43% of people who are diagnosed at an early stage, the 5-year survival rate is 31%.

Pancreatic cancer (PC) is a lethal malignancy with a very high mortality rate. Pancreatic cancer is a group of heterogeneous diseases and includes cancer of the endocrine (islet cell carcinoma, neuroendocrine carcinoma and carcinoma of carcinoid tumors) and exocrine (pancreatic ductal adenocarcinoma and acinar) pancreas. Among these pathologies, pancreatic ductal adenocarcinoma accounts for approximately 90% of all cases. Notably, a significantly better treatment outcome has been reported in cases where a tumor was detected at an early stage.

Table A lists methods currently available for diagnosing pancreatic cancer.

TABLE A
Current Pancreatic Cancer Diagnostic Tests

| Modality | Issues |
|---|---|
| CA19-9 test | Low selectivity and specificity |
| | Detect mostly late stage of cancer |
| | Limited accuracy in identifying patients with small tumors |
| | Not recommended as a screening test |
| CT Scan | Suboptimal for early pancreatic neoplasm |
| | High concern for repeat radiation exposure |
| | High cost |
| MRI/MRCP | High cost |
| | No published data on accuracy or yield |
| ERCP | High cost |
| | Invasive procedure with high risk of pancreatitis |
| | The patient has to be sedated or anaesthetized |

As shown in Table A, detection of pancreatic cancer relies heavily on procedures, notably imaging. Advances in the imaging technology have allowed improved detection of small lesions. However, these advances have also led to increases in false-positive findings, necessitating invasive procedures to make a definitive diagnosis. Given the probability of false-positive findings associated with the CT screening, there is a substantial need for additional test methods to discriminate between benign vs malignant nodules. There are similar challenges in imaging-based screening for other GI malignancies and a high unmet need for highly sensitive and non-invasive diagnostic tests.

More than 2% of adults harbor a pancreatic cyst, a subset of which progresses to invasive lesions with lethal consequences. As the result of the increasing use of imaging technologies in standard medical practice, pancreatic cysts are being identified with an increasing frequency. Management of these cysts is concomitantly becoming a major clinical problem. Cystic lesions occur in more than 20% of patients examined at autopsy, in as many as 19.6% of patients evaluated by MRI, and in as many as 2.6% of patients evaluated by computed tomography. In the vast majority of cases, the cysts are identified as incidental findings in patients undergoing imaging for symptoms unrelated to pancreatic pathology. However, once a cyst is identified, it poses a challenging life-long management problem. Some cyst types are virtually always benign, some are low-grade malignant, and others are precursors to invasive pancreatic ductal adenocarcinomas. The distinction among cyst types is therefore critical for the effective management of patients with pancreatic cysts.

The potential for malignant transformation varies among pancreatic cystic neoplasms (PCN) subtypes. Imaging and a cyst fluid analysis are sometimes used to identify premalignant or malignant cases that should undergo operative resection. Therefore, there is a critical need to develop an efficient and noninvasive liquid biopsy test which can be used to distinguish a patient with a benign, non-premalignant disease from a patient with malignant pancreatic cysts.

A cancer is associated with major changes in biopathways, including upregulation of fucosyltransferases, sialyltransferases, mannosyl (α-1,6-)-glycoprotein β-1,6-N-acetyl-glucosaminyltransferase. Changes in the expression of glycosyltransferases result in altered glycan assembly, which occurs in the endoplasmic reticulum and Golgi. Accordingly, the glycoprotein products of tumor cells carry aberrant carbohydrate structures compared with their normal counterparts. Typical changes include increased levels of fucose and sialic acid, the addition of polylactosamine units and N-acetylglucosamine, and higher-ordered branching of N-linked glycans. O-linked glycans are also affected in cancer, typically carrying incomplete or prematurely truncated structures relative to those found on normal cells. After secretion or proteolytic cleavage, glycosylated molecules and/or their cleavage products can be released into the interstitial space, where they can enter the circulation. (Drake et al. 2010, Clin Chem, 56(2): 223-236)

Tumors produce glycoproteins that carry oligosaccharides with structures that are markedly different from the same protein produced by a normal cell. A single protein can have many glycosylation sites that greatly amplify the signals they generate compared with their protein backbones, thus tumor glycoproteins can serve as cancer biomarkers. The glycosylation machinery appears to be particularly sensitive to malignant transformation; as a result, the saccharide structures that are added to normal cellular proteins change, resulting in neoglycoforms that can be released from the cell through conventional secretory pathways, or as the result of enhanced proteinase activity. (Drake et al. 2010, Clin Chem, 56(2): 223-236)

Carbohydrates and their associated glycoproteins represent a rich, underexplored source of biomarkers. Glycoproteins with complex glycans are membrane bound or secreted. There is a substantial evidence that cancer cells exhibit altered glycans relative to normal cells. The potential of targeting glycoproteins to identify biomarkers was investigated by enriching N-linked glycopeptides from tissues, cells, and plasma and identifying corresponding peptide sequences and proteins by mass spectrometry. A significant overlap was observed between glycoproteins identified in tissues and cells and glycoproteins identified in plasma, leading to the conclusion that extracellular glycoproteins originating from tissues and cells are released into the blood at concentrations that are detectable by mass spectrometry. See U.S. Patent Publication 2007/0099251.

It has been demonstrated that in pancreatic cancer glucose metabolism pathways and glycosylation levels are changing throughout disease progression, specifically on a background of hypoxia. Hypoxia promotes selective pressure on malignant cells that must develop adaptive metabolic responses to reach their energetic and biosynthetic demands. In a mouse model of pancreatic cancer, it was demonstrated that hypoxic areas from pancreatic ductal adenocarcinoma are mainly composed of epithelial cells harboring epithelial-mesenchymal transition features and expressing glycolytic markers, two characteristics associated with tumor aggressiveness. In this model, it has been also shown that hypoxia increases the “glycolytic” switch of pancreatic cancer cells from oxidative phosphorylation to lactate production and demonstrated that increased lactate efflux from hypoxic cancer cells favors the growth of normoxic cancer cells. (Guillaumond et al. 2013, PNAS, 110(10): 3919-3924).

Metabolized glucose and glutamine converge toward a common pathway, termed the hexosamine biosynthetic pathway, which allows O-linked N-acetylglucosamine modifications of proteins. Importantly, it was reported that hypoxia increases transcription of hexosamine biosynthetic pathway genes as well as levels of O-glycosylated proteins and that O-linked N-acetylglucosaminylation of proteins is a process required for hypoxic pancreatic cancer cell survival. Hypoxia-driven metabolic adaptive processes, such as high glycolytic rate and the hexosamine biosynthetic pathway activation, favor hypoxic and normoxic cancer cell survival and correlate with pancreatic cancer aggressiveness. (Guillaumond et al. 2013, PNAS, Mr 5; 110(10): 3919-3924).

In other studies, it was demonstrated that mucins, specifically, MUC1 and MUC4, are differentially glycosylated as the disease progressed from the early stage to metastatic disease. De novo expression of several mucins correlated with increased metastasis, indicating a potentially more invasive tumor phenotype. (Remmers et al. 2013, Clin Cancer Res. April 15: 19(8)).

There remains the need for an accurate and non-invasive test that can be used to detect and monitor a GI cancer.

### SUMMARY

Provided is a method for screening, monitoring and/or treating a gastrointestinal (GI) cancer patient, wherein the GI cancer is selected from the group consisting of colorectal cancer, gastric cancer, liver cancer, and pancreatic cancer. A sample from the patient is obtained and glycosylated proteins are isolated from the sample. The isolated glycoproteins are then analyzed for the presence of any of biomarkers from Tables 1A, 2A, 3A, 4A, 5A, 6A, 7A, and any combination thereof. The presence of at least some of the biomarkers in the sample being indicative a GI cancer. The isolated glycosylated proteins can be also grouped into a profile of pathways, and matched with at least one profile selected from the group of profiles of Tables 1, 2, 3, 4, 5, 6, 7, 8, and any combination thereof. At least a partial match with at least one profile from Tables 1, 2, 3, 4, 5, 6, 7, 8 being indicative of a GI cancer.

The sample can be selected from the group consisting of a human tissue biopsy or biosample including pancreas biopsy sample, gastrointestinal sample, blood sample, plasma sample, serum sample, circulating tumor cells sample, tear sample, saliva sample, sperm sample, urine sample, fecal sample and hair sample. Blood or plasma samples are particularly preferred.

The sample can be analyzed using one or more techniques selected from the group consisting of chromatography, gas chromatography, liquid chromatography, mass spectrometry, ELISA, antibody linkage, immunoassay, biochip assay, microarray, nanoassay, spectroscopy, a multiplex molecular assay or techniques which utilize a fluorescent, enzyme, radioactive, metallic, biotin, chemiluminescent, bioluminescent molecule assay. The sample can be analyzed using a combination of a detection techniques of nucleic acids and proteins or peptides.

In the further embodiments of the method, any of biomarkers of Tables 1A, 1, 2A, 2, 3A, 3, 4A, 4, 5A, 5, 6A, 6, 7A and 7 are immobilized on a solid support.

The method can be conducted by reacting the patient's sample with at least one anybody or protein chemistry based reagent specific to at least one biomarker and/or glycobiomarker of Tables 1A, 1, 2A, 2, 3A, 3, 4A, 4, 5A, 6A, 6, 7A or 7. In further embodiments, the method can be conducted by reacting the patient's sample with a synthetic compound or probe which react with at least one protein specific to at least one biomarker and/or glycobiomarker of Tables 1A, 1, 2A, 2, 3A, 3, 4A, 4, 5A, 6A, 6, 7A or 7.

Further embodiments in provide a panel comprising a profile of biomarkers selected from the group consisting of Tables 1A, 1, 2A, 2, 3A, 3, 4A, 4, 5A, 6A, 6, 7A, 7 or8, and any combination thereof. Kits comprising the panels are provided as well.

Further embodiments provide a method for detecting or monitoring a disorder of the pancreas, the method comprising obtaining a sample from a patient and testing the sample for at least one or more biomarker and/or glycobiomarker selected from Tables 5A, 5, 6A, 6 and 9. The disorder of the pancreas is selected from the group consisting of acute pancreatitis, chronic pancreatitis, hereditary pancreatitis, pancreatic neoplasm, and pancreatic cancer. The testing can be conducted by reacting the patient's sample with at least one anybody or protein chemistry based reagent specific to at least one biomarker and/or glycobiomarker of Tables 5A, 5, 6A, 6 and 9. The testing is conducted by reacting the patient's sample with at least one protein specific to at least one biomarker and/or glycobiomarker of Tables 5A, 5, 6A, 6 and 9. The testing can be also conducted by reacting the patient's sample with a synthetic compound or probe which react with at least one protein specific to at least one biomarker and/or glycobiomarker of Tables 5A, 5, 6A, 6 and 9.

Further embodiments provide a method for treating a disorder of the pancreas, the method comprising obtaining a sample from a mammal in need of the treatment and testing the sample for at least one or more biomarker and/or glycobiomarker selected from Tables 5A, 5, 6A, 6 and 9.

### BRIEF DESCRIPTION OF THE DRAWINGS

FIG. 1 shows the relationship between 33 proteins in the Adherens Junction Assembly (Nectin) pathway. This figure was generated with ELSEVIER PATHWAY STUDIO®.

FIG. 2 shows the relationship between 66 proteins in the Bradykinin Effects in Inflammation pathway. This figure was generated with ELSEVIER PATHWAY STUDIO®.

FIG. 3 shows the relationship between 38 proteins in the coagulation cascade pathway. This figure was generated with ELSEVIER PATHWAY STUDIO®.

FIG. 4 shows the relationship between 25 proteins in the complement activation pathway by lectin. This figure was generated with ELSEVIER PATHWAY STUDIO®.

FIG. 5 shows the relationship between 45 proteins in the complement activation in macular degeneration pathway. This figure was generated with ELSEVIER PATHWAY STUDIO®.

FIG. 6 shows the relationship between 30 proteins in the complement alternative pathway. This figure was generated with ELSEVIER PATHWAY STUDIO®.

FIG. 7 shows the relationship between 29 proteins in the complement cascade activation by pentraxin pathway. This figure was generated with ELSEVIER PATHWAY STUDIO®.

FIG. 8 shows the relationship between 28 proteins in the complement classical pathway. This figure was generated with ELSEVIER PATHWAY STUDIO®.

FIG. 9 shows the relationship between 37 proteins in the focal junction assembly pathway. This figure was generated with ELSEVIER PATHWAY STUDIO®.

FIG. 10 shows the relationship between 36 proteins in the glycolysis pathway. This figure was generated with ELSEVIER PATHWAY STUDIO®.

FIG. 11 shows the relationship between 33 proteins in the histidine-rich glycoprotein (HRG) pathway. This figure was generated with ELSEVIER PATHWAY STUDIO®.

FIG. 12 shows the relationship between 44 proteins in the lipogenesis regulation pathway. This figure was generated with ELSEVIER PATHWAY STUDIO®.

FIG. 13 shows the relationship between 26 proteins in the microtubule cytoskeleton pathway. This figure was generated with ELSEVIER PATHWAY STUDIO®.

FIG. 14 shows the relationship between proteins in the Neutrophil Activation via Adherence on Endothelial Cells pathway. This figure was generated with ELSEVIER PATHWAY STUDIO®.

FIG. 15 shows the relationship between proteins in the Plasmin Effects in Inflammation pathway. This figure was generated with ELSEVIER PATHWAY STUDIO®.

FIG. 16 shows the relationship between proteins in the Platelet Activation via Adhesion Molecules pathway. This figure was generated with ELSEVIER PATHWAY STUDIO®.

FIG. 17 shows the relationship between proteins in the Platelet Activation via GPCR Signaling pathway. This figure was generated with ELSEVIER PATHWAY STUDIO®.

FIG. 18 shows the relationship between proteins in the Positive Acute Phase Proteins Synthesis pathway. This figure was generated with ELSEVIER PATHWAY STUDIO®.

FIG. 19 shows the relationship between proteins in the Protein Folding pathway. This figure was generated with ELSEVIER PATHWAY STUDIO®.

FIG. 20 shows the relationship between proteins in the Scavenger Receptors in Platelet Activation pathway. This figure was generated with ELSEVIER PATHWAY STUDIO®.

FIG. 21 shows the relationship between proteins in the Scavenger Receptors in Platelet Aggregation pathway. This figure was generated with ELSEVIER PATHWAY STUDIO®.

FIG. 22 shows the relationship between proteins in the TAM Receptors in Platelet Aggregation pathway. This figure was generated with ELSEVIER PATHWAY STUDIO®.

FIG. 23 shows the relationship between proteins in the Vascular Endothelial Cell Activation by Blood Coagulation Factors pathway. This figure was generated with ELSEVIER PATHWAY STUDIO®.

### DETAILED DESCRIPTION

This invention provides compositions and methods for detection, screening, monitoring and treatment of gastrointestinal (GI) cancers, including colorectal cancer, gastric cancer, liver cancer, and pancreatic cancer.

Provided is a method by which a patient's protein expression profile is obtained by isolating glycosylated proteins from the patient's liquid biopsy sample. Suitable liquid biopsy samples include blood, plasma, serum or urine. The glycosylated proteins in the profile are grouped into pathways and analyzed for deviations from a profile of a healthy individual. A deviation in a number of the glycosylated proteins in at least one pathway is indicative of the patient's GI cancer. This analysis can be used for developing a treatment plan for a patient with a particular emphasis on using drugs suitable for targeting the affected pathways and/or proteins.

A patient's profile of glycosylated proteins can be also obtained to evaluate results of cancer treatment, including a surgery, chemotherapy, radiation and/or immunotherapy. In this embodiment of the method, a patient's profile of glycosylated proteins after the cancer treatment is comparted to the patient's profile of glycosylated proteins before the cancer treatment. A decrease in the number of abnormally glycosylated proteins means that the treatment is beneficial to the patient. No changes or an increase in the number of abnormally glycosylated proteins means that the treatment plan needs to be modified or cancelled.

A patient's profile of glycosylated proteins can be also obtained to monitor the patient for an onset of a GI cancer. Many patients, including patients with a hereditary history of a GI cancer in a family, can benefit from this procedure which monitors the patient's profile of glycosylated proteins and pathways, and detects any changes in the profile over a period of time.

In one embodiment of the present methods, a patient's profile of glycosylated proteins is prepared by obtaining a blood, plasma, or serum sample from the patient. Glycosylated proteins are then isolated from the sample. Mass spectrometry of protein expression profile is performed to identify the glycosylated proteins in the sample. The patient's profile is then compared to a profile of a healthy individual in order to diagnose a GI cancer. This method can be used to diagnose a GI cancer. In alternative, a patient's profile of glycosylated proteins in a blood, plasma, or serum sample is monitored over a period of time by periodically repeating the analysis in order to detect an early onset of GI cancer or to determine if a particular cancer treatment is beneficial to the patient.

In alternative to the mass spectrometry analysis, a patient's profile of glycosylated proteins and affected pathways can be analyzed with a chip which comprises a set of biomarkers of a GI cancer. In further embodiments, the profiling of glycosylated proteins may comprise identifying affected pathways.

Table 1A discloses glycoproteins differentially expressed in plasma of colorectal cancer (CRC) female patients. These glycoproteins can be used for diagnosing, monitoring and treating a CRC patient by the present methods. In these methods, glycoproteins of Table 1A are used as a set of biomarkers indicative of CRC.

TABLE 1A
Glycoproteins differentially expressed in plasma of CRC female patients

| Identified Proteins (739) | Accession Number |
|---|---|
| 14-3-3 protein sigma OS = Homo sapiens GN = SFN PE = 1 SV = 1 | sp\|P31947\|1433S_HUMAN |
| 14-3-3 protein zeta/delta OS = Homo sapiens GN = YWHAZ PE = 1 SV = 1 | sp\|P63104\|1433Z_HUMAN |
| 78 kDa glucose-regulated protein OS = Homo sapiens GN = HSPA5 PE = 1 SV = 2 | sp\|P11021\|GRP78_HUMAN |
| Actin, cytoplasmic 2 OS = Homo sapiens GN = ACTG1 PE = 1 SV = 1 | sp\|P63261\|ACTG_HUMAN |
| Actin-related protein 2/3 complex subunit 2 OS = Homo sapiens GN = ARPC2 PE = 1 | sp\|O15144\|ARPC2_HUMAN |
| SV = 1 | |
| Adenylyl cyclase-associated protein 1 OS = Homo sapiens GN = CAP1 PE = 1 SV = 5 | sp\|Q01518\|CAP1_HUMAN |
| ADP/ATP translocase 2 OS = Homo sapiens GN = SLC25A5 PE = 1 SV = 7 | sp\|P05141\|ADT2_HUMAN |
| ADP-ribosylation factor 3 OS = Homo sapiens GN = ARF3 PE = 1 SV = 2 | sp\|P61204\|ARF3_HUMAN |
| | (+1) |
| Alpha-1-antitrypsin OS = Homo sapiens GN = SERPINA1 PE = 1 SV = 3 | sp\|P01009\|A1AT_HUMAN |
| Alpha-1B-glycoprotein OS = Homo sapiens GN = A1BG PE = 1 SV = 4 | sp\|P04217\|A1BG_HUMAN |
| Alpha-2-HS-glycoprotein OS = Homo sapiens GN = AHSG PE = 1 SV = 1 | sp\|P02765\|FETUA_HUMAN |
| Alpha-actinin-1 OS = Homo sapiens GN = ACTN1 PE = 1 SV = 2 | sp\|P12814\|ACTN1_HUMAN |
| Alpha-enolase OS = Homo sapiens GN = ENO1 PE = 1 SV = 2 | sp\|P06733\|ENOA_HUMAN |
| Angiotensinogen OS = Homo sapiens GN = AGT PE = 1 SV = 1 | sp\|P01019\|ANGT_HUMAN |
| Apolipoprotein A-I OS = Homo sapiens GN = APOA1 PE = 1 SV = 1 | sp\|P02647\|APOA1_HUMAN |
| Apolipoprotein E OS = Homo sapiens GN = APOE PE = 1 SV = 1 | sp\|P02649\|APOE_HUMAN |
| Apolipoprotein L1 OS = Homo sapiens GN = APOL1 PE = 1 SV = 5 | sp\|O14791\|APOL1_HUMAN |
| Apolipoprotein(a) OS = Homo sapiens GN = LPA PE = 1 SV = 1 | sp\|P08519\|APOA_HUMAN |
| Arachidonate 12-lipoxygenase, 12S-type OS = Homo sapiens GN = ALOX12 PE = 1 SV = 4 | sp\|P18054\|LOX12_HUMAN |
| ATP synthase subunit beta, mitochondrial OS = Homo sapiens GN = ATP5B PE = 1 SV = 3 | sp\|P06576\|ATPB_HUMAN |
| Attractin OS = Homo sapiens GN = ATRN PE = 1 SV = 2 | sp\|O75882\|ATRN_HUMAN |
| Beta-parvin OS = Homo sapiens GN = PARVB PE = 1 SV = 1 | sp\|Q9HBI1\|PARVB_HUMAN |
| Calpain-1 catalytic subunit OS = Homo sapiens GN = CAPN1 PE = 1 SV = 1 | sp\|P07384\|CAN1_HUMAN |
| Carbonic anhydrase 1 OS = Homo sapiens GN = CA1 PE = 1 SV = 2 | sp\|P00915\|CAH1_HUMAN |
| Carboxypeptidase N catalytic chain OS = Homo sapiens GN = CPN1 PE = 1 SV = 1 | sp\|P15169\|CBPN_HUMAN |
| Cathepsin G OS = Homo sapiens GN = CTSG PE = 1 SV = 2 | sp\|P08311\|CATG_HUMAN |
| Clathrin heavy chain 1 OS = Homo sapiens GN = CLTC PE = 1 SV = 5 | sp\|Q00610\|CLH1_HUMAN |
| Clusterin OS = Homo sapiens GN = CLU PE = 1 SV = 1 | sp\|P10909\|CLUS_HUMAN |
| Coagulation factor IX OS = Homo sapiens GN = F9 PE = 1 SV = 2 | sp\|P00740\|FA9_HUMAN |
| Coagulation factor V OS = Homo sapiens GN = F5 PE = 1 SV = 4 | sp\|P12259\|FA5_HUMAN |
| Coagulation factor X OS = Homo sapiens GN = F10 PE = 1 SV = 2 | sp\|P00742\|FA10_HUMAN |
| Coagulation factor XIII B chain OS = Homo sapiens GN = F13B PE = 1 SV = 3 | sp\|P05160\|F13B_HUMAN |
| Complement C1q subcomponent subunit C OS = Homo sapiens GN = C1QC PE = 1 SV = 3 | sp\|P02747\|C1QC_HUMAN |
| Complement C1r subcomponent OS = Homo sapiens GN = C1R PE = 1 SV = 2 | sp\|P00736\|C1R_HUMAN |
| Complement C1s subcomponent OS = Homo sapiens GN = C1S PE = 1 SV = 1 | sp\|P09871\|C1S_HUMAN |
| Complement C2 OS = Homo sapiens GN = C2 PE = 1 SV = 2 | sp\|P06681\|CO2_HUMAN |
| Complement component C9 OS = Homo sapiens GN = C9 PE = 1 SV = 2 | sp\|P02748\|CO9_HUMAN |
| Complement factor B OS = Homo sapiens GN = CFB PE = 1 SV = 2 | sp\|P00751\|CFAB_HUMAN |
| Complement factor I OS = Homo sapiens GN = CFI PE = 1 SV = 2 | sp\|P05156\|CFAI_HUMAN |
| Corticosteroid-binding globulin OS = Homo sapiens GN = SERPINA6 PE = 1 SV = 1 | sp\|P08185\|CBG_HUMAN |
| C-reactive protein OS = Homo sapiens GN = CRP PE = 1 SV = 1 | sp\|P02741\|CRP_HUMAN |
| EGF-containing fibulin-like extracellular matrix protein 1 OS = Homo sapiens | |
| GN = EFEMP1 PE = 1 SV = 2 | sp\|Q12805\|FBLN3_HUMAN |
| Endoplasmin OS = Homo sapiens GN = HSP90B1 PE = 1 SV = 1 | sp\|P14625\|ENPL_HUMAN |
| Erythrocyte band 7 integral membrane protein OS = Homo sapiens GN = STOM PE = 1 | sp\|P27105\|STOM_HUMAN |
| SV = 3 | |
| Ezrin OS = Homo sapiens GN = EZR PE = 1 SV = 4 | sp\|P15311\|EZRI_HUMAN |
| Fermitin family homolog 3 OS = Homo sapiens GN = FERMT3 PE = 1 SV = 1 | sp\|Q86UX7\|URP2_HUMAN |
| Fetuin-B OS = Homo sapiens GN = FETUB PE = 1 SV = 2 | sp\|Q9UGM5\|FETUB_HUMAN |
| Fibrinogen beta chain OS = Homo sapiens GN = FGB PE = 1 SV = 2 | sp\|P02675\|FIBB_HUMAN |
| Fibrinogen gamma chain OS = Homo sapiens GN = FGG PE = 1 SV = 3 | sp\|P02679\|FIBG_HUMAN |
| Fibronectin OS = Homo sapiens GN = FN1 PE = 1 SV = 4 | sp\|P02751\|FINC_HUMAN |
| Filamin-A OS = Homo sapiens GN = FLNA PE = 1 SV = 4 | sp\|P21333\|FLNA_HUMAN |
| Fructose-bisphosphate aldolase A OS = Homo sapiens GN = ALDOA PE = 1 SV = 2 | sp\|P04075\|ALDOA_HUMAN |
| Galectin-3-binding protein OS = Homo sapiens GN = LGALS3BP PE = 1 SV = 1 | sp\|Q08380\|LG3BP_HUMAN |
| Gelsolin OS = Homo sapiens GN = GSN PE = 1 SV = 1 | sp\|P06396\|GELS_HUMAN |
| Glyceraldehyde-3-phosphate dehydrogenase OS = Homo sapiens GN = GAPDH PE = 1 | sp\|P04406\|G3P_HUMAN |
| SV = 3 | |
| Haptoglobin OS = Homo sapiens GN = HP PE = 1 SV = 1 | sp\|P00738\|HPT_HUMAN |
| Haptoglobin-related protein OS = Homo sapiens GN = HPR PE = 2 SV = 2 | sp\|P00739\|HPTR_HUMAN |
| Heat shock cognate 71 kDa protein OS = Homo sapiens GN = HSPA8 PE = 1 SV = 1 | sp\|P11142\|HSP7C_HUMAN |
| Heat shock protein HSP 90-beta OS = Homo sapiens GN = HSP90AB1 PE = 1 SV = 4 | sp\|P08238\|HS90B_HUMAN |
| Hemoglobin subunit alpha OS = Homo sapiens GN = HBA1 PE = 1 SV = 2 | sp\|P69905\|HBA_HUMAN |
| Heterogeneous nuclear ribonucleoprotein K OS = Homo sapiens GN = HNRNPK PE = 1 | sp\|P61978\|HNRPK_HUMAN |
| SV = 1 | |
| Hexokinase-1 OS = Homo sapiens GN = HK1 PE = 1 SV = 3 | sp\|P19367\|HXK1_HUMAN |
| Ig delta chain C region OS = Homo sapiens GN = IGHD PE = 1 SV = 2 | sp\|P01880\|IGHD_HUMAN |
| Ig gamma-1 chain C region OS = Homo sapiens GN = IGHG1 PE = 1 SV = 1 | sp\|P01857\|IGHG1_HUMAN |
| Ig gamma-4 chain C region OS = Homo sapiens GN = IGHG4 PE = 1 SV = 1 | sp\|P01861\|IGHG4_HUMAN |
| Ig heavy chain V-I region HG3 OS = Homo sapiens PE = 3 SV = 1 | sp\|P01743\|HV102_HUMAN |
| Ig heavy chain V-I region WOL OS = Homo sapiens PE = 1 SV = 1 | sp\|P01760\|HV105_HUMAN |
| Ig heavy chain V-II region ARH-77 OS = Homo sapiens PE = 4 SV = 1 | sp\|P06331\|HV209_HUMAN |
| Ig heavy chain V-II region SESS OS = Homo sapiens PE = 2 SV = 1 | sp\|P04438\|HV208_HUMAN |
| Ig heavy chain V-III region BUR OS = Homo sapiens PE = 1 SV = 1 | sp\|P01773\|HV312_HUMAN |
| Ig heavy chain V-III region CAM OS = Homo sapiens PE = 1 SV = 1 | sp\|P01768\|HV307_HUMAN |
| Ig heavy chain V-III region HIL OS = Homo sapiens PE = 1 SV = 1 | sp\|P01771\|HV310_HUMAN |
| Ig heavy chain V-III region NIE OS = Homo sapiens PE = 1 SV = 1 | sp\|P01770\|HV309_HUMAN |
| Ig kappa chain C region OS = Homo sapiens GN = IGKC PE = 1 SV = 1 | sp\|P01834\|IGKC_HUMAN |
| Ig kappa chain V-I region Lay OS = Homo sapiens PE = 1 SV = 1 | sp\|P01605\|KV113_HUMAN |
| Ig kappa chain V-I region Mev OS = Homo sapiens PE = 1 SV = 1 | sp\|P01612\|KV120_HUMAN |
| Ig kappa chain V-II region FR OS = Homo sapiens PE = 1 SV = 1 | sp\|P01615\|KV202_HUMAN |
| Ig kappa chain V-II region MIL OS = Homo sapiens PE = 1 SV = 1 | sp\|P01616\|KV203_HUMAN |
| Ig kappa chain V-II region RPMI 6410 OS = Homo sapiens PE = 4 SV = 1 | sp\|P06310\|KV206_HUMAN |
| Ig kappa chain V-III region CLL OS = Homo sapiens PE = 4 SV = 2 | sp\|P04207\|KV308_HUMAN |
| Ig kappa chain V-III region IARC/BL41 OS = Homo sapiens PE = 4 SV = 1 | sp\|P06311\|KV311_HUMAN |
| Ig kappa chain V-III region NG9 (Fragment) OS = Homo sapiens PE = 2 SV = 1 | sp\|P01621\|KV303_HUMAN |
| Ig lambda chain V region 4A OS = Homo sapiens PE = 4 SV = 1 | sp\|P04211\|LV001_HUMAN |
| Ig lambda chain V-I region HA OS = Homo sapiens PE = 1 SV = 1 | sp\|P01700\|LV102_HUMAN |
| Ig lambda chain V-I region NEW OS = Homo sapiens PE = 1 SV = 1 | sp\|P01701\|LV103_HUMAN |
| Ig lambda chain V-I region NIG-64 OS = Homo sapiens PE = 1 SV = 1 | sp\|P01702\|LV104_HUMAN |
| | (+1) |
| Ig lambda chain V-V region DEL OS = Homo sapiens PE = 1 SV = 1 | sp\|P01719\|LV501_HUMAN |
| Insulin-like growth factor-binding protein complex acid labile subunit OS = | sp\|P35858\|ALS_HUMAN |
| Homo sapiens GN = IGFALS PE = 1 SV = 1 | |
| Integrin alpha-IIb OS = Homo sapiens GN = ITGA2B PE = 1 SV = 3 | sp\|P08514\|ITA2B_HUMAN |
| Integrin beta-3 OS = Homo sapiens GN = ITGB3 PE = 1 SV = 2 | sp\|P05106\|ITB3_HUMAN |
| Integrin-linked protein kinase OS = Homo sapiens GN = ILK PE = 1 SV = 2 | sp\|Q13418\|ILK_HUMAN |
| Inter-alpha-trypsin inhibitor heavy chain H1 OS = Homo sapiens GN = ITIH1 PE = 1 | sp\|P19827\|ITIH1_HUMAN |
| SV = 3 | |
| Isocitrate dehydrogenase [NADP], mitochondrial OS = Homo sapiens GN = IDH2 PE = 1 | sp\|P48735\|IDHP_HUMAN |
| SV = 2 | |
| Kallistatin OS = Homo sapiens GN = SERPINA4 PE = 1 SV = 3 | sp\|P29622\|KAIN_HUMAN |
| Kininogen-1 OS = Homo sapiens GN = KNG1 PE = 1 SV = 2 | sp\|P01042\|KNG1_HUMAN |
| Leucine-rich alpha-2-glycoprotein OS = Homo sapiens GN = LRG1 PE = 1 SV = 2 | sp\|P02750\|A2GL_HUMAN |
| LIM and senescent cell antigen-like-containing domain protein 1 OS = Homo sapiens | sp\|P48059\|LIMS1_HUMAN |
| GN = LIMS1 PE = 1 SV = 4 | |
| Lipopolysaccharide-binding protein OS = Homo sapiens GN = LBP PE = 1 SV = 3 | sp\|P18428\|LBP_HUMAN |
| L-lactate dehydrogenase A chain OS = Homo sapiens GN = LDHA PE = 1 SV = 2 | sp\|P00338\|LDHA_HUMAN |
| L-lactate dehydrogenase B chain OS = Homo sapiens GN = LDHB PE = 1 SV = 2 | sp\|P07195\|LDHB_HUMAN |
| Multimerin-1 OS = Homo sapiens GN = MMRN1 PE = 1 SV = 3 | sp\|Q13201\|MMRN1_HUMAN |
| Myosin light polypeptide 6 OS = Homo sapiens GN = MYL6 PE = 1 SV = 2 | sp\|P60660\|MYL6_HUMAN |
| Myosin-9 OS = Homo sapiens GN = MYH9 PE = 1 SV = 4 | sp\|P35579\|MYH9_HUMAN |
| Peptidyl-prolyl cis-trans isomerase B OS = Homo sapiens GN = PPIB PE = 1 SV = 2 | sp\|P23284\|PPIB_HUMAN |
| Phosphate carrier protein, mitochondrial OS = Homo sapiens GN = SLC25A3 PE = 1 | sp\|Q00325\|MPCP_HUMAN |
| SV = 2 | |
| Plasma protease C1 inhibitor OS = Homo sapiens GN = SERPING1 PE = 1 SV = 2 | sp\|P05155\|IC1_HUMAN |
| Plasma serine protease inhibitor OS = Homo sapiens GN = SERPINA5 PE = 1 SV = 3 | sp\|P05154\|IPSP_HUMAN |
| Platelet glycoprotein Ib alpha chain OS = Homo sapiens GN = GP1BA PE = 1 SV = 2 | sp\|P07359\|GP1BA_HUMAN |
| Platelet glycoprotein Ib beta chain OS = Homo sapiens GN = GP1BB PE = 1 SV = 1 | sp\|P13224\|GP1BB_HUMAN |
| Pleckstrin OS = Homo sapiens GN = PLEK PE = 1 SV = 3 | sp\|P08567\|PLEK_HUMAN |
| Profilin-1 OS = Homo sapiens GN = PFN1 PE = 1 SV = 2 | sp\|P07737\|PROF1_HUMAN |
| Protein disulfide-isomerase A3 OS = Homo sapiens GN = PDIA3 PE = 1 SV = 4 | sp\|P30101\|PDIA3_HUMAN |
| Proteoglycan 4 OS = Homo sapiens GN = PRG4 PE = 1 SV = 2 | sp\|Q92954\|PRG4_HUMAN |
| Purine nucleoside phosphorylase OS = Homo sapiens GN = PNP PE = 1 SV = 2 | sp\|P00491\|PNPH_HUMAN |
| Putative V-set and immunoglobulin domain-containing-like protein IGHV4OR15-8 | sp\|A6NJ16\|IV4F8_HUMAN |
| OS = Homo sapiens GN = IGHV4OR15-8 PE = 5 SV = 2 | |
| Pyruvate kinase PKM OS = Homo sapiens GN = PKM PE = 1 SV = 4 | sp\|P14618\|KPYM_HUMAN |
| Ras-related protein Rab-10 OS = Homo sapiens GN = RAB10 PE = 1 SV = 1 | sp\|P61026\|RAB10_HUMAN |
| Ras-related protein Rap-1b OS = Homo sapiens GN = RAP1B PE = 1 SV = 1 | sp\|P61224\|RAP1B_HUMAN |
| Reticulon-4 OS = Homo sapiens GN = RTN4 PE = 1 SV = 2 | sp\|Q9NQC3\|RTN4_HUMAN |
| Sarcoplasmic/endoplasmic reticulum calcium ATPase 3 OS = Homo sapiens | sp\|Q93084\|AT2A3_HUMAN |
| GN = ATP2A3 PE = 1 SV = 2 | |
| Serum amyloid P-component OS = Homo sapiens GN = APCS PE = 1 SV = 2 | sp\|P02743\|SAMP_HUMAN |
| Solute carrier family 2, facilitated glucose transporter member 3 OS = Homo sapiens | sp\|P11169\|GTR3_HUMAN |
| GN = SLC2A3 PE = 1 SV = 1 | |
| Talin-1 OS = Homo sapiens GN = TLN1 PE = 1 SV = 3 | sp\|Q9Y490\|TLN1_HUMAN |
| Thrombospondin-1 OS = Homo sapiens GN = THBS1 PE = 1 SV = 2 | sp\|P07996\|TSP1_HUMAN |
| Transgelin-2 OS = Homo sapiens GN = TAGLN2 PE = 1 SV = 3 | sp\|P37802\|TAGL2_HUMAN |
| Transthyretin OS = Homo sapiens GN = TTR PE = 1 SV = 1 | sp\|P02766\|TTHY_HUMAN |
| Tubulin alpha-1B chain OS = Homo sapiens GN = TUBA1B PE = 1 SV = 1 | sp\|P68363\|TBA1B_HUMAN |
| Tubulin alpha-4A chain OS = Homo sapiens GN = TUBA4A PE = 1 SV = 1 | sp\|P68366\|TBA4A_HUMAN |
| Tubulin beta-1 chain OS = Homo sapiens GN = TUBB1 PE = 1 SV = 1 | sp\|Q9H4B7\|TBB1_HUMAN |
| Tubulin beta-2A chain OS = Homo sapiens GN = TUBB2A PE = 1 SV = 1 | sp\|Q13885\|TBB2A_HUMAN |
| Vinculin OS = Homo sapiens GN = VCL PE = 1 SV = 4 | sp\|P18206\|VINC_HUMAN |
| Vitamin D-binding protein OS = Homo sapiens GN = GC PE = 1 SV = 1 | sp\|P02774\|VTDB_HUMAN |
| Vitronectin OS = Homo sapiens GN = VTN PE = 1 SV = 1 | sp\|P04004\|VTNC_HUMAN |
| Voltage-dependent anion-selective channel protein 3 OS = Homo sapiens | sp\|Q9Y277\|VDAC3_HUMAN |
| GN = VDAC3 PE = 1 SV = 1 | |
| von Willebrand factor OS = Homo sapiens GN = VWF PE = 1 SV = 4 | sp\|P04275\|VWF_HUMAN |
| WD repeat-containing protein 1 OS = Homo sapiens GN = WDR1 PE = 1 SV = 4 | sp\|O75083\|WDR1_HUMAN |

Table 1 provides a profile of abnormalities detected in pathways and glycosylated proteins in plasma of colorectal cancer female patients. This profile, including any of protein biomarkers and pathways, can be used for diagnosing, monitoring and treating a CRC patient by the present methods. In these methods, the profile of Table 1 is used as a set of biomarkers indicative of CRC.

TABLE 1
A profile of abnormalities in pathways and glycosylated proteins in a CRC female patient.

| | | | Jaccard |
|---|---|---|---|
| Pathway | Glycosylated Proteins | p-value | similarity |
| Complement Activation in | CFB, CFI, C2, C1R, C1S, C1QC, | 1.11151E−8 | 4.97512E−2 |
| Macular Degeneration | C9, CRP, VTN, CLU | | |
| Focal Junction Assembly | ACTN1, FLNA, FN1, TLN1, ILK, | 1.66465E−8 | 4.87805E−2 |
| | VCL, CAPN1, VTN, LIMS1, | | |
| | ITGB3 | | |
| Platelet Activation via Adhesion | GP1BA, FGB, FGG, ARPC2, | 3.16888E−6 | 3.58423E−2 |
| Molecules | VCL, VWF, ITGA2B, ITGB3, | | |
| | TLN1, GP1BB | | |
| Glycolysis | HK1, GAPDH, ENO1, PKM, | 1.60626E−5 | 3.40136E−2 |
| | ALDOA | | |
| TAM Receptors in Platelet | FGB, FGG, VWF, ITGB3 | 9.25453E−5 | 2.87770E−2 |
| Aggregation | | | |
| Scavenger Receptors in Platelet | GP1BA, APOA1, APOE, | 9.76831E−5 | 3.10881E−2 |
| Activation | ITGA2B, ITGB3, GP1BB | | |
| Complement Cascade Activation | CFB, APCS, C2, C1S, C9, CRP | 1.26998E−4 | 3.04569E−2 |
| by Pentraxins | | | |
| Complement Classical Pathway | C2, C1R, C1S, C1QC, C9 | 1.29618E−4 | 3.01205E−2 |
| Coagulation Cascade | FGB, FGG, KNG1, F5, F9, F10 | 1.83636E−4 | 2.95567E−2 |
| Scavenger Receptors in Platelet | FGB, FGG, TLN1, ITGA2B, ITGB3 | 1.96450E−4 | 2.92398E−2 |
| Aggregation | | | |

Table 2A discloses glycoproteins differentially expressed in plasma of colorectal cancer (CRC) male patients. These glycoproteins can be used for diagnosing, monitoring and treating a CRC patient by the present methods. In these methods, glycoproteins of Table 2A are used as a set of biomarkers indicative of CRC.

TABLE 2A
Glycoproteins differentially expressed in plasma of CRC male patients

| Identified Proteins (739) | Accession Number |
|---|---|
| 14-3-3 protein zeta/delta OS = Homo sapiens GN = YWHAZ PE = 1 SV = 1 | sp\|P63104\|1433Z_HUMAN |
| Actin, cytoplasmic 2 OS = Homo sapiens GN = ACTG1 PE = 1 SV = 1 | sp\|P63261\|ACTG_HUMAN |
| Adenylyl cyclase-associated protein 1 OS = Homo sapiens GN = CAP1 PE = 1 SV = 5 | sp\|Q01518\|CAP1_HUMAN |
| ADP/ATP translocase 2 OS = Homo sapiens GN = SLC25A5 PE = 1 SV = 7 | sp\|P05141\|ADT2_HUMAN |
| Alpha-1B-glycoprotein OS = Homo sapiens GN = A1BG PE = 1 SV = 4 | sp\|P04217\|A1BG_HUMAN |
| Alpha-actinin-1 OS = Homo sapiens GN = ACTN1 PE = 1 SV = 2 | sp\|P12814\|ACTN1_HUMAN |
| Apolipoprotein B-100 OS = Homo sapiens GN = APOB PE = 1 SV = 2 | sp\|P04114\|APOB_HUMAN |
| Apolipoprotein E OS = Homo sapiens GN = APOE PE = 1 SV = 1 | sp\|P02649\|APOE_HUMAN |
| Apolipoprotein L1 OS = Homo sapiens GN = APOL1 PE = 1 SV = 5 | sp\|O14791\|APOL1_HUMAN |
| ATP synthase subunit alpha, mitochondrial OS = Homo sapiens GN = ATP5A1 | sp\|P25705\|ATPA_HUMAN |
| PE = 1 SV = 1 | |
| ATP synthase subunit beta, mitochondrial OS = Homo sapiens GN = ATP5B PE = 1 | sp\|P06576\|ATPB_HUMAN |
| SV = 3 | |
| Beta-2-glycoprotein 1 OS = Homo sapiens GN = APOH PE = 1 SV = 3 | sp\|P02749\|APOH_HUMAN |
| C4b-binding protein beta chain OS = Homo sapiens GN = C4BPB PE = 1 SV = 1 | sp\|P20851\|C4BPB_HUMAN |
| Cholesteryl ester transfer protein OS = Homo sapiens GN = CETP PE = 1 SV = 2 | sp\|P11597\|CETP_HUMAN |
| Clathrin heavy chain 1 OS = Homo sapiens GN = CLTC PE = 1 SV = 5 | sp\|Q00610\|CLH1_HUMAN |
| Coagulation factor IX OS = Homo sapiens GN = F9 PE = 1 SV = 2 | sp\|P00740\|FA9_HUMAN |
| Coagulation factor XI OS = Homo sapiens GN = F11 PE = 1 SV = 1 | sp\|P03951\|FA11_HUMAN |
| Coagulation factor XII OS = Homo sapiens GN = F12 PE = 1 SV = 3 | sp\|P00748\|FA12_HUMAN |
| Coagulation factor XIII A chain OS = Homo sapiens GN = F13A1 PE = 1 SV = 4 | sp\|P00488\|F13A_HUMAN |
| Complement C1q subcomponent subunit A OS = Homo sapiens GN = C1QA PE = 1 | sp\|P02745\|C1QA_HUMAN |
| SV = 2 | |
| Complement C1r subcomponent OS = Homo sapiens GN = C1R PE = 1 SV = 2 | sp\|P00736\|C1R_HUMAN |
| Complement factor B OS = Homo sapiens GN = CFB PE = 1 SV = 2 | sp\|P00751\|CFAB_HUMAN |
| Corticosteroid-binding globulin OS = Homo sapiens GN = SERPINA6 PE = 1 SV = 1 | sp\|P08185\|CBG_HUMAN |
| C-reactive protein OS = Homo sapiens GN = CRP PE = 1 SV = 1 | sp\|P02741\|CRP_HUMAN |
| Desmoplakin OS = Homo sapiens GN = DSP PE = 1 SV = 3 | sp\|P15924\|DESP_HUMAN |
| EGF-containing fibulin-like extracellular matrix protein 1 OS = Homo sapiens | sp\|Q12805\|FBLN3_HUMAN |
| GN = EFEMP1 PE = 1 SV = 2 | |
| Erythrocyte band 7 integral membrane protein OS = Homo sapiens GN = STOM | sp\|P27105\|STOM_HUMAN |
| PE = 1 SV = 3 | |
| Fermitin family homolog 3 OS = Homo sapiens GN = FERMT3 PE = 1 SV = 1 | sp\|Q86UX7\|URP2_HUMAN |
| Fibrinogen alpha chain OS = Homo sapiens GN = FGA PE = 1 SV = 2 | sp\|P02671\|FIBA_HUMAN |
| Fibrinogen beta chain OS = Homo sapiens GN = FGB PE = 1 SV = 2 | sp\|P02675\|FIBB_HUMAN |
| Fibrinogen gamma chain OS = Homo sapiens GN = FGG PE = 1 SV = 3 | sp\|P02679\|FIBG_HUMAN |
| Fibronectin OS = Homo sapiens GN = FN1 PE = 1 SV = 4 | sp\|P02751\|FINC_HUMAN |
| Fibulin-1 OS = Homo sapiens GN = FBLN1 PE = 1 SV = 4 | sp\|P23142\|FBLN1_HUMAN |
| Filamin-A OS = Homo sapiens GN = FLNA PE = 1 SV = 4 | sp\|P21333\|FLNA_HUMAN |
| Gelsolin OS = Homo sapiens GN = GSN PE = 1 SV = 1 | sp\|P06396\|GELS_HUMAN |
| Glutathione peroxidase 3 OS = Homo sapiens GN = GPX3 PE = 1 SV = 2 | sp\|P22352\|GPX3_HUMAN |
| Glyceraldehyde-3-phosphate dehydrogenase OS = Homo sapiens GN = GAPDH | sp\|P04406\|G3P_HUMAN |
| PE = 1 SV = 3 | |
| Guanine nucleotide-binding protein G(k) subunit alpha OS = Homo sapiens | sp\|P08754\|GNAI3_HUMAN |
| GN = GNAI3 PE = 1 SV = 3 | |
| Heat shock cognate 71 kDa protein OS = Homo sapiens GN = HSPA8 PE = 1 SV = 1 | sp\|P11142\|HSP7C_HUMAN |
| Heat shock protein HSP 90-beta OS = Homo sapiens GN = HSP90AB1 PE = 1 SV = 4 | sp\|P08238\|HS90B_HUMAN |
| Hepatocyte growth factor-like protein OS = Homo sapiens GN = MST1 PE = 1 SV = 2 | sp\|P26927\|HGFL_HUMAN |
| Histone H4 OS = Homo sapiens GN = HIST1H4A PE = 1 SV = 2 | sp\|P62805\|H4_HUMAN |
| Hyaluronan-binding protein 2 OS = Homo sapiens GN = HABP2 PE = 1 SV = 1 | sp\|Q14520\|HABP2_HUMAN |
| Ig alpha-1 chain C region OS = Homo sapiens GN = IGHA1 PE = 1 SV = 2 | sp\|P01876\|IGHA1_HUMAN |
| Ig alpha-2 chain C region OS = Homo sapiens GN = IGHA2 PE = 1 SV = 3 | sp\|P01877\|IGHA2_HUMAN |
| Ig delta chain C region OS = Homo sapiens GN = IGHD PE = 1 SV = 2 | sp\|P01880\|IGHD_HUMAN |
| Ig heavy chain V-I region 5 (Fragment) OS = Homo sapiens GN = IGKV1-5 PE = 4 | sp\|P01602\|KV110_HUMAN |
| SV = 2 | |
| Ig heavy chain V-I region EU OS = Homo sapiens PE = 1 SV = 1 | sp\|P01742\|HV101_HUMAN |
| Ig heavy chain V-I region V35 OS = Homo sapiens PE = 1 SV = 1 | sp\|P23083\|HV103_HUMAN |
| Ig heavy chain V-II region ARH-77 OS = Homo sapiens PE = 4 SV = 1 | sp\|P06331\|HV209_HUMAN |
| Ig heavy chain V-II region NEWM OS = Homo sapiens PE = 1 SV = 1 | sp\|P01825\|HV207_HUMAN |
| Ig heavy chain V-II region SESS OS = Homo sapiens PE = 2 SV = 1 | sp\|P04438\|HV208_HUMAN |
| Ig heavy chain V-II region WAH OS = Homo sapiens PE = 1 SV = 1 | sp\|P01824\|HV206_HUMAN |
| Ig heavy chain V-III region GA OS = Homo sapiens PE = 1 SV = 1 | sp\|P01769\|HV308_HUMAN |
| Ig heavy chain V-III region KOL OS = Homo sapiens PE = 1 SV = 1 | sp\|P01772\|HV311_HUMAN |
| Ig heavy chain V-III region NIE OS = Homo sapiens PE = 1 SV = 1 | sp\|P01770\|HV309_HUMAN |
| Ig heavy chain V-III region TIL OS = Homo sapiens PE = 1 SV = 1 | sp\|P01765\|HV304_HUMAN |
| Ig heavy chain V-III region WEA OS = Homo sapiens PE = 1 SV = 1 | sp\|P01763\|HV302_HUMAN |
| Ig kappa chain C region OS = Homo sapiens GN = IGKC PE = 1 SV = 1 | sp\|P01834\|IGKC_HUMAN |
| Ig kappa chain V-I region DEE OS = Homo sapiens PE = 1 SV = 1 | sp\|P01597\|KV105_HUMAN |
| Ig kappa chain V-I region Lay OS = Homo sapiens PE = 1 SV = 1 | sp\|P01605\|KV113_HUMAN |
| Ig kappa chain V-I region WEA OS = Homo sapiens PE = 1 SV = 1 | sp\|P01610\|KV118_HUMAN |
| Ig kappa chain V-II region MIL OS = Homo sapiens PE = 1 SV = 1 | sp\|P01616\|KV203_HUMAN |
| Ig kappa chain V-II region TEW OS = Homo sapiens PE = 1 SV = 1 | sp\|P01617\|KV204_HUMAN |
| Ig kappa chain V-III region IARC/BL41 OS = Homo sapiens PE = 4 SV = 1 | sp\|P06311\|KV311_HUMAN |
| Ig kappa chain V-III region VG (Fragment) OS = Homo sapiens PE = 1 SV = 1 | sp\|P04433\|KV309_HUMAN |
| Ig lambda chain V region 4A OS = Homo sapiens PE = 4 SV = 1 | sp\|P04211\|LV001_HUMAN |
| Ig lambda chain V-I region HA OS = Homo sapiens PE = 1 SV = 1 | sp\|P01700\|LV102_HUMAN |
| Ig lambda chain V-I region NEW OS = Homo sapiens PE = 1 SV = 1 | sp\|P01701\|LV103_HUMAN |
| Ig lambda chain V-I region NEWM OS = Homo sapiens PE = 1 SV = 1 | sp\|P01703\|LV105_HUMAN |
| Ig lambda chain V-I region WAH OS = Homo sapiens PE = 1 SV = 1 | sp\|P04208\|LV106_HUMAN |
| Ig lambda chain V-III region LOI OS = Homo sapiens PE = 1 SV = 1 | sp\|P80748\|LV302_HUMAN |
| Ig lambda chain V-III region SH OS = Homo sapiens PE = 1 SV = 1 | sp\|P01714\|LV301_HUMAN |
| Ig lambda chain V-IV region Hil OS = Homo sapiens PE = 1 SV = 1 | sp\|P01717\|LV403_HUMAN |
| IgGFc-binding protein OS = Homo sapiens GN = FCGBP PE = 1 SV = 3 | sp\|Q9Y6R7\|FCGBP_HUMAN |
| Insulin-like growth factor-binding protein complex acid labile subunit | sp\|P35858\|ALS_HUMAN |
| OS = Homo sapiens GN = IGFALS PE = 1 SV = 1 | |
| Integrin alpha-IIb OS = Homo sapiens GN = ITGA2B PE = 1 SV = 3 | sp\|P08514\|ITA2B_HUMAN |
| Integrin beta-3 OS = Homo sapiens GN = ITGB3 PE = 1 SV = 2 | sp\|P05106\|ITB3_HUMAN |
| Inter-alpha-trypsin inhibitor heavy chain H1 OS = Homo sapiens GN = ITIH1 PE = 1 | sp\|P19827\|ITIH1_HUMAN |
| SV = 3 | |
| Inter-alpha-trypsin inhibitor heavy chain H2 OS = Homo sapiens GN = ITIH2 PE = 1 | sp\|P19823\|ITIH2_HUMAN |
| SV = 2 | |
| Inter-alpha-trypsin inhibitor heavy chain H4 OS = Homo sapiens GN = ITIH4 PE = 1 | sp\|Q14624\|ITIH4_HUMAN |
| SV = 4 | |
| Isocitrate dehydrogenase [NADP], mitochondrial OS = Homo sapiens GN = IDH2 | sp\|P48735\|IDHP_HUMAN |
| PE = 1 SV = 2 | |
| Junction plakoglobin OS = Homo sapiens GN = JUP PE = 1 SV = 3 | sp\|P14923\|PLAK_HUMAN |
| Keratinocyte proline-rich protein OS = Homo sapiens GN = KPRP PE = 1 SV = 1 | sp\|Q5T749\|KPRP_HUMAN |
| Leucine-rich alpha-2-glycoprotein OS = Homo sapiens GN = LRG1 PE = 1 SV = 2 | sp\|P02750\|A2GL_HUMAN |
| Lipopolysaccharide-binding protein OS = Homo sapiens GN = LBP PE = 1 SV = 3 | sp\|P18428\|LBP_HUMAN |
| Multimerin-1 OS = Homo sapiens GN = MMRN1 PE = 1 SV = 3 | sp\|Q13201\|MMRN1_HUMAN |
| Myeloperoxidase OS = Homo sapiens GN = MPO PE = 1 SV = 1 | sp\|P05164\|PERM_HUMAN |
| Myosin-9 OS = Homo sapiens GN = MYH9 PE = 1 SV = 4 | sp\|P35579\|MYH9_HUMAN |
| Peptidyl-prolyl cis-trans isomerase B OS = Homo sapiens GN = PPIB PE = 1 SV = 2 | sp\|P23284\|PPIB_HUMAN |
| Phosphate carrier protein, mitochondrial OS = Homo sapiens GN = SLC25A3 PE = 1 | sp\|Q00325\|MPCP_HUMAN |
| SV = 2 | |
| Phosphatidylinositol-glycan-specific phospholipase D OS = Homo sapiens | sp\|P80108\|PHLD_HUMAN |
| GN = GPLD1 PE = 1 SV = 3 | |
| Phospholipid transfer protein OS = Homo sapiens GN = PLTP PE = 1 SV = 1 | sp\|P55058\|PLTP_HUMAN |
| Pleckstrin OS = Homo sapiens GN = PLEK PE = 1 SV = 3 | sp\|P08567\|PLEK_HUMAN |
| Pregnancy zone protein OS = Homo sapiens GN = PZP PE = 1 SV = 4 | sp\|P20742\|PZP_HUMAN |
| Profilin-1 OS = Homo sapiens GN = PFN1 PE = 1 SV = 2 | sp\|P07737\|PROF1_HUMAN |
| Protein disulfide-isomerase A3 OS = Homo sapiens GN = PDIA3 PE = 1 SV = 4 | sp\|P30101\|PDIA3_HUMAN |
| Protein S100-A8 OS = Homo sapiens GN = S100A8 PE = 1 SV = 1 | sp\|P05109\|S10A8_HUMAN |
| Protein Z-dependent protease inhibitor OS = Homo sapiens GN = SERPINA10 PE = 1 | sp\|Q9UK55\|ZPI_HUMAN |
| SV = 1 | |
| Proteoglycan 4 OS = Homo sapiens GN = PRG4 PE = 1 SV = 2 | sp\|Q92954\|PRG4_HUMAN |
| Pyruvate kinase PKM OS = Homo sapiens GN = PKM PE = 1 SV = 4 | sp\|P14618\|KPYM_HUMAN |
| Ras-related protein Rab-10 OS = Homo sapiens GN = RAB10 PE = 1 SV = 1 | sp\|P61026\|RAB10_HUMAN |
| Ras-related protein Rap-1b OS = Homo sapiens GN = RAP1B PE = 1 SV = 1 | sp\|P61224\|RAP1B_HUMAN |
| Sarcoplasmic/endoplasmic reticulum calcium ATPase 3 OS = Homo sapiens | sp\|Q93084\|AT2A3_HUMAN |
| GN = ATP2A3 PE = 1 SV = 2 | |
| Serum paraoxonase/arylesterase 1 OS = Homo sapiens GN = PON1 PE = 1 SV = 3 | sp\|P27169\|PON1_HUMAN |
| Sex hormone-binding globulin OS = Homo sapiens GN = SHBG PE = 1 SV = 2 | sp\|P04278\|SHBG_HUMAN |
| Small proline-rich protein 2D OS = Homo sapiens GN = SPRR2D PE = 2 SV = 2 | sp\|P22532\|SPR2D_HUMAN |
| Solute carrier family 2, facilitated glucose transporter member 3 OS = Homo | sp\|P11169\|GTR3_HUMAN |
| sapiens GN = SLC2A3 PE = 1 SV = 1 | |
| Talin-1 OS = Homo sapiens GN = TLN1 PE = 1 SV = 3 | sp\|Q9Y490\|TLN1_HUMAN |
| Thrombospondin-1 OS = Homo sapiens GN = THBS1 PE = 1 SV = 2 | sp\|P07996\|TSP1_HUMAN |
| Thyroxine-binding globulin OS = Homo sapiens GN = SERPINA7 PE = 1 SV = 2 | sp\|P05543\|THBG_HUMAN |
| Transthyretin OS = Homo sapiens GN = TTR PE = 1 SV = 1 | sp\|P02766\|TTHY_HUMAN |
| Tubulin alpha-1B chain OS = Homo sapiens GN = TUBA1B PE = 1 SV = 1 | sp\|P68363\|TBA1B_HUMAN |
| Tubulin beta chain OS = Homo sapiens GN = TUBB PE = 1 SV = 2 | sp\|P07437\|TBB5_HUMAN |
| Tubulin beta-1 chain OS = Homo sapiens GN = TUBB1 PE = 1 SV = 1 | sp\|Q9H4B7\|TBB1_HUMAN |
| Tubulin beta-2A chain OS = Homo sapiens GN = TUBB2A PE = 1 SV = 1 | sp\|Q13885\|TBB2A_HUMAN |
| Vinculin OS = Homo sapiens GN = VCL PE = 1 SV = 4 | sp\|P18206\|VINC_HUMAN |
| Voltage-dependent anion-selective channel protein 3 OS = Homo sapiens | sp\|Q9Y277\|VDAC3_HUMAN |
| GN = VDAC3 PE = 1 SV = 1 | |
| von Willebrand factor OS = Homo sapiens GN = VWF PE = 1 SV = 4 | sp\|P04275\|VWF_HUMAN |
| WD repeat-containing protein 1 OS = Homo sapiens GN = WDR1 PE = 1 SV = 4 | sp\|O75083\|WDR1_HUMAN |

Table 2 provides a profile of abnormalities in pathways and glycosylated proteins in a colorectal cancer male patient. This profile, including any of protein biomarkers and pathways, can be used for diagnosing, monitoring and treating a CRC patient by the present methods. In these methods, the profile of Table 2 is used as a set of biomarkers indicative of CRC.

TABLE 2
A profile of abnormalities in pathways and glycosylated proteins in a CRC
male patient.

| | Glycosylated | | Jaccard |
|---|---|---|---|
| Pathway | Proteins | p-value | similarity |
| Scavanger Receptors in Platelet | APOB, FGA, FGB, | 1.08924E−7 | 4.66667E−2 |
| Aggregation | FGG, TLN1, | | |
| | ITGA2B, ITGB3 | | |
| Coagulation Cascade | FGA, FGB, FGG, F9, | 1.41766E−7 | 4.39560E−2 |
| | F11, F12, F13A1, | | |
| | SERPINA10 | | |
| TAM Receptors in Platelet | FGA, FGB, FGG, | 7.49345E−7 | 4.20168E−2 |
| Aggregation | VWF, ITGB3 | | |
| Platelet Activation via Adhesion | FGA, FGB, FGG, | 1.56396E−5 | 3.05344E−2 |
| Molecules | VCL, VWF, | | |
| | ITGA2B, ITGB3, | | |
| | TLN1 | | |
| Focal Junction Assembly | ACTN1, FLNA, | 4.36457E−5 | 3.15789E−2 |
| | FN1, TLN1, VCL, | | |
| | ITGB3 | | |
| Histidine-Rich Glycoprotein (HRG) | FGA, FGB, FGG, | 2.85537E−4 | 2.60870E−2 |
| | C1QA, THBS1, F12 | | |
| Platelet Activation via GPCR | FGA, FGB, FGG, | 4.94723E−4 | 2.44898E−2 |
| Signaling | ITGB3, TLN1, | | |
| | GNAI3 | | |
| Plasmin Effects in Inflammation | FGA, FGB, FGG, | 1.66747E−3 | 1.97183E−2 |
| | FN1, C1QA, C1R, | | |
| | F12 | | |
| Protein Folding | TUBB2A, TUBB, | 1.75381E−3 | 2.22222E−2 |
| | TUBA1B, TUBB1, | | |
| | HSPA8 | | |
| Scavenger Receptors in Platelet | APOB, APOE, | 2.10901E−3 | 2.27273E−2 |
| Activation | ITGA2B, ITGB3 | | |

Table 3A discloses glycoproteins differentially expressed in plasma of gastric cancer female patients. These glycoproteins can be used for diagnosing, monitoring and treating a gastric cancer patient by the present methods. In these methods, glycoproteins of Table 3A are used as a set of biomarkers indicative of gastric cancer.

TABLE 3A
Glycoproteins differentially expressed in plasma of gastric cancer female
patients

| 14-3-3 protein sigma OS = Homo sapiens GN = SFN PE = 1 SV = 1 | sp\|P31947\|1433S_HUMAN |
|---|---|
| 14-3-3 protein zeta/delta OS = Homo sapiens GN = YWHAZ PE = 1 SV = 1 | sp\|P63104\|1433Z_HUMAN |
| 78 kDa glucose-regulated protein OS = Homo sapiens GN = HSPA5 PE = 1 SV = 2 | sp\|P11021\|GRP78_HUMAN |
| Actin, cytoplasmic 2 OS = Homo sapiens GN = ACTG1 PE = 1 SV = 1 | sp\|P63261\|ACTG_HUMAN |
| Actin-related protein 2/3 complex subunit 1B OS = Homo sapiens GN = ARPC1B PE = 1 | sp\|O15143\|ARC1B_HUMAN |
| SV = 3 | |
| Actin-related protein 2/3 complex subunit 4 OS = Homo sapiens GN = ARPC4 PE = 1 | sp\|P59998\|ARPC4_HUMAN |
| SV = 3 | |
| Adenylyl cyclase-associated protein 1 OS = Homo sapiens GN = CAP1 PE = 1 SV = 5 | sp\|Q01518\|CAP1_HUMAN |
| ADP/ATP translocase 2 OS = Homo sapiens GN = SLC25A5 PE = 1 SV = 7 | sp\|P05141\|ADT2_HUMAN |
| ADP-ribosylation factor 3 OS = Homo sapiens GN = ARF3 PE = 1 SV = 2 | sp\|P61204\|ARF3_HUMAN (+1) |
| Alpha-2-antiplasmin OS = Homo sapiens GN = SERPINF2 PE = 1 SV = 3 | sp\|P08697\|A2AP_HUMAN |
| Alpha-2-macroglobulin OS = Homo sapiens GN = A2M PE = 1 SV = 3 | sp\|P01023\|A2MG_HUMAN |
| Alpha-actinin-1 OS = Homo sapiens GN = ACTN1 PE = 1 SV = 2 | sp\|P12814\|ACTN1_HUMAN |
| Alpha-enolase OS = Homo sapiens GN = ENO1 PE = 1 SV = 2 | sp\|P06733\|ENOA_HUMAN |
| Apolipoprotein A-I OS = Homo sapiens GN = APOA1 PE = 1 SV = 1 | sp\|P02647\|APOA1_HUMAN |
| Apolipoprotein L1 OS = Homo sapiens GN = APOL1 PE = 1 SV = 5 | sp\|O14791\|APOL1_HUMAN |
| Apolipoprotein M OS = Homo sapiens GN = APOM PE = 1 SV = 2 | sp\|O95445\|APOM_HUMAN |
| Apolipoprotein(a) OS = Homo sapiens GN = LPA PE = 1 SV = 1 | sp\|P08519\|APOA_HUMAN |
| ATP synthase subunit alpha, mitochondrial OS = Homo sapiens GN = ATP5A1 PE = 1 | sp\|P25705\|ATPA_HUMAN |
| SV = 1 | |
| Beta-1,4-galactosyltransferase 1 OS = Homo sapiens GN = B4GALT1 PE = 1 SV = 5 | sp\|P15291\|B4GT1_HUMAN |
| Beta-2-glycoprotein 1 OS = Homo sapiens GN = APOH PE = 1 SV = 3 | sp\|P02749\|APOH_HUMAN |
| Beta-parvin OS = Homo sapiens GN = PARVB PE = 1 SV = 1 | sp\|Q9HB11\|PARVB_HUMAN |
| Carbonic anhydrase 1 OS = Homo sapiens GN = CA1 PE = 1 SV = 2 | sp\|P00915\|CAH1_HUMAN |
| Carboxypeptidase B2 OS = Homo sapiens GN = CPB2 PE = 1 SV = 2 | sp\|Q961Y4\|CBPB2_HUMAN |
| Carboxypeptidase N catalytic chain OS = Homo sapiens GN = CPN1 PE = 1 SV = 1 | sp\|P15169\|CBPN_HUMAN |
| Carboxypeptidase N subunit 2 OS = Homo sapiens GN = CPN2 PE = 1 SV = 3 | sp\|P22792\|CPN2_HUMAN |
| Cartilage acidic protein 1 OS = Homo sapiens GN = CRTAC1 PE = 1 SV = 2 | sp\|Q9NQ79\|CRAC1_HUMAN |
| Cholinesterase OS = Homo sapiens GN = BCHE PE = 1 SV = 1 | sp\|P06276\|CHLE_HUMAN |
| Clathrin heavy chain 1 OS = Homo sapiens GN = CLTC PE = 1 SV = 5 | sp\|Q00610\|CLH1_HUMAN |
| Clusterin OS = Homo sapiens GN = CLU PE = 1 SV = 1 | sp\|P10909\|CLUS_HUMAN |
| Coagulation factor IX OS = Homo sapiens GN = F9 PE = 1 SV = 2 | sp\|P00740\|FA9_HUMAN |
| Coagulation factor V OS = Homo sapiens GN = F5 PE = 1 SV = 4 | sp\|P12259\|FA5_HUMAN |
| Coagulation factor X OS = Homo sapiens GN = F10 PE = 1 SV = 2 | sp\|P00742\|FA10_HUMAN |
| Coagulation factor XI OS = Homo sapiens GN = F11 PE = 1 SV = 1 | sp\|P03951\|FA11_HUMAN |
| Coagulation factor XII OS = Homo sapiens GN = F12 PE = 1 SV = 3 | sp\|P00748\|FA12_HUMAN |
| Coagulation factor XIII A chain OS = Homo sapiens GN = F13A1 PE = 1 SV = 4 | sp\|P00488\|F13A_HUMAN |
| Complement C1q subcomponent subunit A OS = Homo sapiens GN = C1QA PE = 1 | sp\|P02745\|C1QA_HUMAN |
| SV = 2 | |
| Complement C1r subcomponent OS = Homo sapiens GN = C1R PE = 1 SV = 2 | sp\|P00736\|C1R_HUMAN |
| Complement C3 OS = Homo sapiens GN = C3 PE = 1 SV = 2 | sp\|P01024\|CO3_HUMAN |
| Complement component C8 beta chain OS = Homo sapiens GN = C8B PE = 1 SV = 3 | sp\|P07358\|C08B_HUMAN |
| Complement factor H-related protein 1 OS = Homo sapiens GN = CFHR1 PE = 1 SV = 2 | sp\|Q03591\|FHR1_HUMAN |
| Complement factor H-related protein 5 OS = Homo sapiens GN = CFHR5 PE = 1 SV = 1 | sp\|Q9BXR6\|FHR5_HUMAN |
| Complement factor I OS = Homo sapiens GN = CFI PE = 1 SV = 2 | sp\|P05156\|CFAI_HUMAN |
| C-reactive protein OS = Homo sapiens GN = CRP PE = 1 SV = 1 | sp\|P02741\|CRP_HUMAN |
| Dolichyl-diphosphooligosaccharide--protein glycosyltransferase 48 kDa subunit | sp\|P39656\|OST48_HUMAN |
| OS = Homo sapiens GN = DDOST PE = 1 SV = 4 | |
| EGF-containing fibulin-like extracellular matrix protein 1 OS = Homo sapiens | sp\|Q12805\|FBLN3_HUMAN |
| GN = EFEMP1 PE = 1 SV = 2 | |
| Endoplasmin OS = Homo sapiens GN = HSP90B1 PE = 1 SV = 1 | sp\|P14625\|ENPL_HUMAN |
| Erythrocyte band 7 integral membrane protein OS = Homo sapiens GN = STOM PE = 1 | sp\|P27105\|STOM_HUMAN |
| SV = 3 | |
| Ezrin OS = Homo sapiens GN = EZR PE = 1 SV = 4 | sp\|P15311\|EZRI_HUMAN |
| F-actin-capping protein subunit beta OS = Homo sapiens GN = CAPZB PE = 1 SV = 4 | sp\|P47756\|CAPZB_HUMAN |
| Fermitin family homolog 3 OS = Homo sapiens GN = FERMT3 PE = 1 SV = 1 | sp\|Q86UX7\|URP2_HUMAN |
| Fetuin-B OS = Homo sapiens GN = FETUB PE = 1 SV = 2 | sp\|Q9UGM5\|FETUB_HUMAN |
| Fibulin-1 OS = Homo sapiens GN = FBLN1 PE = 1 SV = 4 | sp\|P23142\|FBLN1_HUMAN |
| Filamin-A OS = Homo sapiens GN = FLNA PE = 1 SV = 4 | sp\|P21333\|FLNA_HUMAN |
| Fructose-bisphosphate aldolase A OS = Homo sapiens GN = ALDOA PE = 1 SV = 2 | sp\|P04075\|ALDOA_HUMAN |
| Galectin-3-binding protein OS = Homo sapiens GN = LGALS3BP PE = 1 SV = 1 | sp\|Q08380\|LG3BP_HUMAN |
| Gelsolin OS = Homo sapiens GN = GSN PE = 1 SV = 1 | sp\|P06396\|GELS_HUMAN |
| Glutathione peroxidase 3 OS = Homo sapiens GN = GPX3 PE = 1 SV = 2 | sp\|P22352\|GPX3_HUMAN |
| Glyceraldehyde-3-phosphate dehydrogenase OS = Homo sapiens GN = GAPDH PE = 1 | sp\|P04406\|G3P_HUMAN |
| SV = 3 | |
| Haptoglobin OS = Homo sapiens GN = HP PE = 1 SV = 1 | sp\|P00738\|HPT_HUMAN |
| Haptoglobin-related protein OS = Homo sapiens GN = HPR PE = 2 SV = 2 | sp\|P00739\|HPTR_HUMAN |
| Heat shock cognate 71 kDa protein OS = Homo sapiens GN = HSPA8 PE = 1 SV = 1 | sp\|P11142\|HSP7C_HUMAN |
| Heat shock protein HSP 90-beta OS = Homo sapiens GN = HSP90AB1 PE = 1 SV = 4 | sp\|P08238\|HS90B_HUMAN |
| Hemoglobin subunit alpha OS = Homo sapiens GN = HBA1 PE = 1 SV = 2 | sp\|P69905\|HBA_HUMAN |
| Hemoglobin subunit beta OS = Homo sapiens GN = HBB PE = 1 SV = 2 | sp\|P68871\|HBB_HUMAN |
| Heparin cofactor 2 OS = Homo sapiens GN = SERPIND1 PE = 1 SV = 3 | sp\|P05546\|HEP2_HUMAN |
| Hepatocyte growth factor-like protein OS = Homo sapiens GN = MST1 PE = 1 SV = 2 | sp\|P26927\|HGFL_HUMAN |
| Histidine-rich glycoprotein OS = Homo sapiens GN = HRG PE = 1 SV = 1 | sp\|P04196\|HRG_HUMAN |
| Identified Proteins (739) | Accession Number |
| Ig gamma-4 chain C region OS = Homo sapiens GN = IGHG4 PE = 1 SV = 1 | sp\|P01861\|IGHG4_HUMAN |
| Ig heavy chain V-I region EU OS = Homo sapiens PE = 1 SV = 1 | sp\|P01742\|HV101_HUMAN |
| Ig heavy chain V-I region HG3 OS = Homo sapiens PE = 3 SV = 1 | sp\|P01743\|HV102_HUMAN |
| Ig heavy chain V-I region WOL OS = Homo sapiens PE = 1 SV = 1 | sp\|P01760\|HV105_HUMAN |
| Ig heavy chain V-II region ARH-77 OS = Homo sapiens PE = 4 SV = 1 | sp\|P06331\|HV209_HUMAN |
| Ig heavy chain V-II region WAH OS = Homo sapiens PE = 1 SV = 1 | sp\|P01824\|HV206_HUMAN |
| Ig heavy chain V-III region 23 OS = Homo sapiens GN = IGHV3-23 PE = 1 SV = 2 | sp\|P01764\|HV303_HUMAN |
| Ig heavy chain V-III region BRO OS = Homo sapiens PE = 1 SV = 1 | sp\|P01766\|HV305_HUMAN |
| Ig heavy chain V-III region BUT OS = Homo sapiens PE = 1 SV = 1 | sp\|P01767\|HV306_HUMAN |
| Ig heavy chain V-III region GAL OS = Homo sapiens PE = 1 SV = 1 | sp\|P01781\|HV320_HUMAN |
| Ig heavy chain V-III region NIE OS = Homo sapiens PE = 1 SV = 1 | sp\|P01770\|HV309_HUMAN |
| Ig heavy chain V-III region WEA OS = Homo sapiens PE = 1 SV = 1 | sp\|P01763\|HV302_HUMAN |
| Ig kappa chain C region OS = Homo sapiens GN = IGKC PE = 1 SV = 1 | sp\|P01834\|IGKC_HUMAN |
| Ig kappa chain V-I region CAR OS = Homo sapiens PE = 1 SV = 1 | sp\|P01596\|KV104_HUMAN |
| Ig kappa chain V-I region Mev OS = Homo sapiens PE = 1 SV = 1 | sp\|P01612\|KV120_HUMAN |
| Ig kappa chain V-II region FR OS = Homo sapiens PE = 1 SV = 1 | sp\|P01615\|KV202_HUMAN |
| Ig kappa chain V-II region RPMI 6410 OS = Homo sapiens PE = 4 SV = 1 | sp\|P06310\|KV206_HUMAN |
| Ig kappa chain V-IV region STH (Fragment) OS = Homo sapiens PE = 1 SV = 1 | sp\|P83593\|KV405_HUMAN |
| Ig lambda chain V region 4A OS = Homo sapiens PE = 4 SV = 1 | sp\|P04211\|LV001_HUMAN |
| Ig lambda chain V-I region HA OS = Homo sapiens PE = 1 SV = 1 | sp\|P01700\|LV102_HUMAN |
| Ig lambda chain V-I region NEWM OS = Homo sapiens PE = 1 SV = 1 | sp\|P01703\|LV105_HUMAN |
| Ig lambda chain V-I region NIG-64 OS = Homo sapiens PE = 1 SV = 1 | sp\|P01702\|LV104_HUMAN (+1) |
| Ig lambda chain V-II region TRO OS = Homo sapiens PE = 1 SV = 1 | sp\|P01707\|LV204_HUMAN |
| IgGFc-binding protein OS = Homo sapiens GN = FCGBP PE = 1 SV = 3 | sp\|Q9Y6R7\|FCGBP_HUMAN |
| Integrin alpha-IIb OS = Homo sapiens GN = ITGA2B PE = 1 SV = 3 | sp\|P08514\|ITA2B_HUMAN |
| Integrin beta-3 OS = Homo sapiens GN = ITGB3 PE = 1 SV = 2 | sp\|P05106\|ITB3_HUMAN |
| Integrin-linked protein kinase OS = Homo sapiens GN = ILK PE = 1 SV = 2 | sp\|Q13418\|ILK_HUMAN |
| Inter-alpha-trypsin inhibitor heavy chain H1 OS = Homo sapiens GN = ITIH1 PE = 1 | sp\|P19827\|ITIH1_HUMAN |
| SV = 3 | |
| Inter-alpha-trypsin inhibitor heavy chain H3 OS = Homo sapiens GN = ITIH3 PE = 1 | sp\|Q06033\|ITIH3_HUMAN |
| SV = 2 | |
| Inter-alpha-trypsin inhibitor heavy chain H4 OS = Homo sapiens GN = ITIH4 PE = 1 | sp\|Q14624\|ITIH4_HUMAN |
| SV = 4 | |
| Isocitrate dehydrogenase [NADP], mitochondrial OS = Homo sapiens GN = IDH2 PE = 1 | sp\|P48735\|IDHP_HUMAN |
| SV = 2 | |
| Junction plakoglobin OS = Homo sapiens GN = JUP PE = 1 SV = 3 | sp\|P14923\|PLAK_HUMAN |
| Keratin, type I cytoskeletal 9 OS = Homo sapiens GN = KRT9 PE = 1 SV = 3 | sp\|P35527\|K1C9_HUMAN |
| Keratin, type II cytoskeletal 1 OS = Homo sapiens GN = KRT1 PE = 1 SV = 6 | sp\|P04264\|K2C1_HUMAN |
| Keratin, type II cytoskeletal 2 epidermal OS = Homo sapiens GN = KRT2 PE = 1 SV = 2 | sp\|P35908\|K22E_HUMAN |
| Kininogen-1 OS = Homo sapiens GN = KNG1 PE = 1 SV = 2 | sp\|P01042\|KNG1_HUMAN |
| LIM and senescent cell antigen-like-containing domain protein 1 OS = Homo sapiens | sp\|P48059\|LIMS1_HUMAN |
| GN = LIMS1 PE = 1 SV = 4 | |
| L-lactate dehydrogenase A chain OS = Homo sapiens GN = LDHA PE = 1 SV = 2 | sp\|P00338\|LDHA_HUMAN |
| Lysozyme C OS = Homo sapiens GN = LYZ PE = 1 SV = 1 | sp\|P61626\|LYSC_HUMAN |
| Multimerin-1 OS = Homo sapiens GN = MMRN1 PE = 1 SV = 3 | sp\|Q13201\|MMRN1_HUMAN |
| Myosin-9 OS = Homo sapiens GN = MYH9 PE = 1 SV = 4 | sp\|P35579\|MYH9_HUMAN |
| N-acetylmuramoyl-L-alanine amidase OS = Homo sapiens GN = PGLYRP2 PE = 1 SV = 1 | sp\|Q96PD5\|PGRP2_HUMAN |
| Peptidyl-prolyl cis-trans isomerase B OS = Homo sapiens GN = PPIB PE = 1 SV = 2 | sp\|P23284\|PPIB_HUMAN |
| Peroxiredoxin-2 OS = Homo sapiens GN = PRDX2 PE = 1 SV = 5 | sp\|P32119\|PRDX2_HUMAN |
| Peroxiredoxin-6 OS = Homo sapiens GN = PRDX6 PE = 1 SV = 3 | sp\|P30041\|PRDX6_HUMAN |
| Phosphate carrier protein, mitochondrial OS = Homo sapiens GN = SLC25A3 PE = 1 | sp\|Q00325\|MPCP_HUMAN |
| SV = 2 | |
| Pigment epithelium-derived factor OS = Homo sapiens GN = SERPINF1 PE = 1 SV = 4 | sp\|P36955\|PEDF_HUMAN |
| Plasma serine protease inhibitor OS = Homo sapiens GN = SERPINA5 PE = 1 SV = 3 | sp\|P05154\|IPSP_HUMAN |
| Plastin-2 OS = Homo sapiens GN = LCP1 PE = 1 SV = 6 | sp\|P13796\|PLSL_HUMAN |
| Platelet glycoprotein Ib alpha chain OS = Homo sapiens GN = GP1BA PE = 1 SV = 2 | sp\|P07359\|GP1BA_HUMAN |
| Platelet glycoprotein Ib beta chain OS = Homo sapiens GN = GP1BB PE = 1 SV = 1 | sp\|P13224\|GP1BB_HUMAN |
| Pleckstrin OS = Homo sapiens GN = PLEK PE = 1 SV = 3 | sp\|P08567\|PLEK_HUMAN |
| Pregnancy zone protein OS = Homo sapiens GN = PZP PE = 1 SV = 4 | sp\|P20742\|PZP_HUMAN |
| Prenylcysteine oxidase 1 OS = Homo sapiens GN = PCYOX1 PE = 1 SV = 3 | sp\|Q9UHG3\|PCYOX_HUMAN |
| Profilin-1 OS = Homo sapiens GN = PFN1 PE = 1 SV = 2 | sp\|P07737\|PROF1_HUMAN |
| Protein broad-minded OS = Homo sapiens GN = TBC1D32 PE = 2 SV = 4 | sp\|Q96NH3\|BROMI_HUMAN |
| Protein disulfide-isomerase A3 OS = Homo sapiens GN = PDIA3 PE = 1 SV = 4 | sp\|P30101\|PDIA3_HUMAN |
| Protein S100-A8 OS = Homo sapiens GN = S100A8 PE = 1 SV = 1 | sp\|P05109\|S10A8_HUMAN |
| Protein S100-A9 OS = Homo sapiens GN = S100A9 PE = 1 SV = 1 | sp\|P06702\|S10A9_HUMAN |
| Proto-oncogene tyrosine-protein kinase Src OS = Homo sapiens GN = SRC PE = 1 SV = 3 | sp\|P12931\|SRC_HUMAN |
| Putative V-set and immunoglobulin domain-containing-like protein IGHV4OR15-8 | sp\|A6NJ16\|IV4F8_HUMAN |
| OS = Homo sapiens GN = IGHV4OR15-8 PE = 5 SV = 2 | |
| Pyruvate kinase PKM OS = Homo sapiens GN = PKM PE = 1 SV = 4 | sp\|P14618\|KPYM_HUMAN |
| Ras-related protein Rab-10 OS = Homo sapiens GN = RAB10 PE = 1 SV = 1 | sp\|P61026\|RAB10_HUMAN |
| Ras-related protein Rap-1b OS = Homo sapiens GN = RAP1B PE = 1 SV = 1 | sp\|P61224\|RAP1B_HUMAN |
| Retinol-binding protein 4 OS = Homo sapiens GN = RBP4 PE = 1 SV = 3 | sp\|P02753\|RET4_HUMAN |
| Sarcoplasmic/endoplasmic reticulum calcium ATPase 3 OS = Homo sapiens | sp\|Q93084\|AT2A3_HUMAN |
| GN = ATP2A3 PE = 1 SV = 2 | |
| Serum amyloid P-component OS = Homo sapiens GN = APCS PE = 1 SV = 2 | sp\|P02743\|SAMP_HUMAN |
| Small proline-rich protein 2D OS = Homo sapiens GN = SPRR2D PE = 2 SV = 2 | sp\|P22532\|SPR2D_HUMAN |
| Talin-1 OS = Homo sapiens GN = TLN1 PE = 1 SV = 3 | sp\|Q9Y490\|TLN1_HUMAN |
| Telomere length regulation protein TEL2 homolog OS = Homo sapiens GN = TELO2 | sp\|Q9Y4R8\|TELO2_HUMAN |
| PE = 1 SV = 2 | |
| Thrombospondin-1 OS = Homo sapiens GN = THBS1 PE = 1 SV = 2 | sp\|P07996\|TSP1_HUMAN |
| Transthyretin OS = Homo sapiens GN = TTR PE = 1 SV = 1 | sp\|P02766\|TTHY_HUMAN |
| Tubulin alpha-1B chain OS = Homo sapiens GN = TUBA1B PE = 1 SV = 1 | sp\|P68363\|TBA1B_HUMAN |
| Tubulin alpha-4A chain OS = Homo sapiens GN = TUBA4A PE = 1 SV = 1 | sp\|P68366\|TBA4A_HUMAN |
| Tubulin beta chain OS = Homo sapiens GN = TUBB PE = 1 SV = 2 | sp\|P07437\|TBB5_HUMAN |
| Tubulin beta-1 chain OS = Homo sapiens GN = TUBB1 PE = 1 SV = 1 | sp\|Q9H4B7\|TBB1_HUMAN |
| Tubulin beta-2A chain OS = Homo sapiens GN = TUBB2A PE = 1 SV = 1 | sp\|Q13885\|TBB2A_HUMAN |
| Ubiquitin-60S ribosomal protein L40 OS = Homo sapiens GN = UBA52 PE = 1 SV = 2 | sp\|P62987\|RL40_HUMAN |
| Vinculin OS = Homo sapiens GN = VCL PE = 1 SV = 4 | sp\|P18206\|VINC_HUMAN |
| Voltage-dependent anion-selective channel protein 3 OS = Homo sapiens | sp\|Q9Y277\|VDAC3_HUMAN |
| GN = VDAC3 PE = 1 SV = 1 | |
| von Willebrand factor OS = Homo sapiens GN = VWF PE = 1 SV = 4 | sp\|P04275\|VWF_HUMAN |
| WD repeat-containing protein 1 OS = Homo sapiens GN = WDR1 PE = 1 SV = 4 | sp\|O75083\|WDR1_HUMAN |

Table 3 provides a profile of abnormalities in pathways and glycosylated proteins in a gastric cancer female patient. This profile, including any of protein biomarkers and pathways, can be used for diagnosing, monitoring and treating a gastric cancer patient by the present methods. In these methods, the profile of Table 3 is used as a set of biomarkers indicative of gastric cancer.

TABLE 3
A profile of abnormalities in pathways and glycosylated proteins in a gastric
cancer female patient.

| | | | Jaccard |
|---|---|---|---|
| Pathway | Glycosylated Proteins | p-value | similarity |
| Complement Activation in | CFI, C1QA, C3, C1R, C8B, | 3.77674E−6 | 3.63636E−2 |
| Macular Degeneration | CRP, CLU, CFHR1 | | |
| Focal Junction Assembly | SRC, ACTN1, FLNA, | 5.15265E−6 | 3.57143E−2 |
| | TLN1, ILK, VCL, LIMS1, | | |
| | ITGB3 | | |
| Platelet Activation via Adhesion | GP1BA, SRC, ARPC4, | 6.77864E−6 | 3.37838E−2 |
| Molecules | ARPC1B, VCL, VWF, | | |
| | ITGA2B, ITGB3, TLN1, | | |
| | GP1BB | | |
| Coagulation Cascade | KNG1, F5, F9, F11, F10, | 3.29309E−5 | 3.19635E−2 |
| | F12, F13A1 | | |
| Adherens Junction Assembly | SRC, ARPC4, ARPC1B, | 1.91850E−4 | 2.73224E−2 |
| (Nectin) | ACTN1, VCL | | |
| Protein Folding | TUBB2A, TUBA4A, | 3.15167E−4 | 2.70270E−2 |
| | TUBB, TUBA1B, TUBB1, | | |
| | HSPA8, HSP90B1 | | |
| Glycolysis | GAPDH, ENO1, PKM, | 4.01213E−4 | 2.42424E−2 |
| | ALDOA | | |
| Neutrophil Activation via | ARPC4, ARPC1B, ACTN1, | 5.85006E−4 | 2.57511E−2 |
| Adherence on Endothelial Cells | TLN1, VCL, ITGB3 | | |
| Scavenger Receptors in Platelet | GP1BA, APOA1, ITGA2B, | 1.29150E−3 | 2.36967E−2 |
| Activation | ITGB3, GP1BB | | |
| Microtubule Cytoskeleton | TUBB2A, TUBA4A, | 1.29150E−3 | 2.36967E−2 |
| | TUBB, TUBA1B, TUBB1 | | |

Table 4A discloses glycoproteins differentially expressed in plasma of gastric cancer male patients. These glycoproteins can be used for diagnosing, monitoring and treating a gastric cancer patient by the present methods. In these methods, glycoproteins of Table 4A are used as a set of biomarkers indicative of gastric cancer.

TABLE 4A
Glycoproteins differentially expressed in plasma of gastric male patients

| Identified Proteins (739) | Accession Number |
|---|---|
| 14-3-3 protein zeta/delta OS = Homo sapiens GN = YWHAZ PE = 1 SV = 1 | sp\|P63104\|1433Z_HUMAN |
| Actin, cytoplasmic 2 OS = Homo sapiens GN = ACTG1 PE = 1 SV = 1 | sp\|P63261\|ACTG_HUMAN |
| Adenylyl cyclase-associated protein 1 OS = Homo sapiens GN = CAP1 PE = 1 | sp\|Q01518\|CAP1_HUMAN |
| SV = 5 | |
| ADP/ATP translocase 2 OS = Homo sapiens GN = SLC25A5 PE = 1 SV = 7 | sp\|P05141\|ADT2_HUMAN |
| Afamin OS = Homo sapiens GN = AFM PE = 1 SV = 1 | sp\|P43652\|AFAM_HUMAN |
| Alpha-1B-glycoprotein OS = Homo sapiens GN = A1BG PE = 1 SV = 4 | sp\|P04217\|A1BG_HUMAN |
| Alpha-2-antiplasmin OS = Homo sapiens GN = SERPINF2 PE = 1 SV = 3 | sp\|P08697\|A2AP_HUMAN |
| Alpha-2-macroglobulin OS = Homo sapiens GN = A2M PE = 1 SV = 3 | sp\|P01023\|A2MG_HUMAN |
| Alpha-actinin-1 OS = Homo sapiens GN = ACTN1 PE = 1 SV = 2 | sp\|P12814\|ACTN1_HUMAN |
| Alpha-enolase OS = Homo sapiens GN = ENO1 PE = 1 SV = 2 | sp\|P06733\|ENOA_HUMAN |
| Angiotensinogen OS = Homo sapiens GN = AGT PE = 1 SV = 1 | sp\|P01019\|ANGT_HUMAN |
| Antithrombin-III OS = Homo sapiens GN = SERPINC1 PE = 1 SV = 1 | sp\|P01008\|ANT3_HUMAN |
| Apolipoprotein B-100 OS = Homo sapiens GN = APOB PE = 1 SV = 2 | sp\|P04114\|APOB_HUMAN |
| Apolipoprotein D OS = Homo sapiens GN = APOD PE = 1 SV = 1 | sp\|P05090\|APOD_HUMAN |
| Apolipoprotein E OS = Homo sapiens GN = APOE PE = 1 SV = 1 | sp\|P02649\|APOE_HUMAN |
| Apolipoprotein(a) OS = Homo sapiens GN = LPA PE = 1 SV = 1 | sp\|P08519\|APOA_HUMAN |
| ATP synthase subunit alpha, mitochondrial OS = Homo sapiens GN = ATP5A1 | sp\|P25705\|ATPA_HUMAN |
| PE = 1 SV = 1 | |
| ATP synthase subunit beta, mitochondrial OS = Homo sapiens GN = ATP5B | sp\|P06576\|ATPB_HUMAN |
| PE = 1 SV = 3 | |
| C4b-binding protein beta chain OS = Homo sapiens GN = C4BPB PE = 1 SV = 1 | sp\|P20851\|C4BPB_HUMAN |
| Calpain-1 catalytic subunit OS = Homo sapiens GN = CAPN1 PE = 1 SV = 1 | sp\|P07384\|CAN1_HUMAN |
| Carbonic anhydrase 1 OS = Homo sapiens GN = CA1 PE = 1 SV = 2 | sp\|P00915\|CAH1_HUMAN |
| Carboxypeptidase N catalytic chain OS = Homo sapiens GN = CPN1 PE = 1 SV = 1 | sp\|P15169\|CBPN_HUMAN |
| Carboxypeptidase N subunit 2 OS = Homo sapiens GN = CPN2 PE = 1 SV = 3 | sp\|P22792\|CPN2_HUMAN |
| Ceruloplasmin OS = Homo sapiens GN = CP PE = 1 SV = 1 | sp\|P00450\|CERU_HUMAN |
| Clathrin heavy chain 1 OS = Homo sapiens GN = CLTC PE = 1 SV = 5 | sp\|Q00610\|CLH1_HUMAN |
| Clusterin OS = Homo sapiens GN = CLU PE = 1 SV = 1 | sp\|P10909\|CLUS_HUMAN |
| Coagulation factor V OS = Homo sapiens GN = F5 PE = 1 SV = 4 | sp\|P12259\|FA5_HUMAN |
| Coagulation factor X OS = Homo sapiens GN = F10 PE = 1 SV = 2 | sp\|P00742\|FA10_HUMAN |
| Coagulation factor XII OS = Homo sapiens GN = F12 PE = 1 SV = 3 | sp\|P00748\|FA12_HUMAN |
| Coagulation factor XIII A chain OS = Homo sapiens GN = F13A1 PE = 1 SV = 4 | sp\|P00488\|F13A_HUMAN |
| Coagulation factor XIII B chain OS = Homo sapiens GN = F13B PE = 1 SV = 3 | sp\|P05160\|F13B_HUMAN |
| Complement C1q subcomponent subunit A OS = Homo sapiens GN = C1QA | sp\|P02745\|C1QA_HUMAN |
| PE = 1 SV = 2 | |
| Complement C1q subcomponent subunit B OS = Homo sapiens GN = C1QB | sp\|P02746\|C1QB_HUMAN |
| PE = 1 SV = 3 | |
| Complement C1s subcomponent OS = Homo sapiens GN = C1S PE = 1 SV = 1 | sp\|P09871\|C1S_HUMAN |
| Complement C2 OS = Homo sapiens GN = C2 PE = 1 SV = 2 | sp\|P06681\|CO2_HUMAN |
| Complement C3 OS = Homo sapiens GN = C3 PE = 1 SV = 2 | sp\|P01024\|CO3_HUMAN |
| Complement C4-A OS = Homo sapiens GN = C4A PE = 1 SV = 2 | sp\|P0C0L4\|CO4A_HUMAN |
| Complement C4-B OS = Homo sapiens GN = C4B PE = 1 SV = 2 | sp\|P0C0L5\|CO4B_HUMAN |
| Complement C5 OS = Homo sapiens GN = C5 PE = 1 SV = 4 | sp\|P01031\|CO5_HUMAN |
| Complement component C6 OS = Homo sapiens GN = C6 PE = 1 SV = 3 | sp\|P13671\|CO6_HUMAN |
| Complement component C7 OS = Homo sapiens GN = C7 PE = 1 SV = 2 | sp\|P10643\|CO7_HUMAN |
| Complement component C8 alpha chain OS = Homo sapiens GN = C8A PE = 1 | sp\|P07357\|C08A_HUMAN |
| SV = 2 | |
| Complement component C8 beta chain OS = Homo sapiens GN = C8B PE = 1 | sp\|P07358\|C08B_HUMAN |
| SV = 3 | |
| Complement factor H OS = Homo sapiens GN = CFH PE = 1 SV = 4 | sp\|P08603\|CFAH_HUMAN |
| Complement factor H-related protein 1 OS = Homo sapiens GN = CFHR1 PE = 1 | sp\|Q03591\|FHR1_HUMAN |
| SV = 2 | |
| Complement factor I OS = Homo sapiens GN = CFI PE = 1 SV = 2 | sp\|P05156\|CFAI_HUMAN |
| Corticosteroid-binding globulin OS = Homo sapiens GN = SERPINA6 PE = 1 SV = 1 | sp\|P08185\|CBG_HUMAN |
| C-reactive protein OS = Homo sapiens GN = CRP PE = 1 SV = 1 | sp\|P02741\|CRP_HUMAN |
| Desmoplakin OS = Homo sapiens GN = DSP PE = 1 SV = 3 | sp\|P15924\|DESP_HUMAN |
| Endoplasmin OS = Homo sapiens GN = HSP90B1 PE = 1 SV = 1 | sp\|P14625\|ENPL_HUMAN |
| Erythrocyte band 7 integral membrane protein OS = Homo sapiens | sp\|P27105\|STOM_HUMAN |
| GN = STOM PE = 1 SV = 3 | |
| Ezrin OS = Homo sapiens GN = EZR PE = 1 SV = 4 | sp\|P15311\|EZRI_HUMAN |
| Fermitin family homolog 3 OS = Homo sapiens GN = FERMT3 PE = 1 SV = 1 | sp\|Q86UX7\|URP2_HUMAN |
| Filamin-A OS = Homo sapiens GN = FLNA PE = 1 SV = 4 | sp\|P21333\|FLNA_HUMAN |
| Fructose-bisphosphate aldolase A OS = Homo sapiens GN = ALDOA PE = 1 SV = 2 | sp\|P04075\|ALDOA_HUMAN |
| Gelsolin OS = Homo sapiens GN = GSN PE = 1 SV = 1 | sp\|P06396\|GELS_HUMAN |
| Glutathione peroxidase 3 OS = Homo sapiens GN = GPX3 PE = 1 SV = 2 | sp\|P22352\|GPX3_HUMAN |
| Glyceraldehyde-3-phosphate dehydrogenase OS = Homo sapiens | sp\|P04406\|G3P_HUMAN |
| GN = GAPDH PE = 1 SV = 3 | |
| Heat shock cognate 71 kDa protein OS = Homo sapiens GN = HSPA8 PE = 1 | sp\|P11142\|HSP7C_HUMAN |
| SV = 1 | |
| Heat shock protein HSP 90-beta OS = Homo sapiens GN = HSP90AB1 PE = 1 | sp\|P08238\|HS90B_HUMAN |
| SV = 4 | |
| Hemoglobin subunit beta OS = Homo sapiens GN = HBB PE = 1 SV = 2 | sp\|P68871\|HBB_HUMAN |
| Hemopexin OS = Homo sapiens GN = HPX PE = 1 SV = 2 | sp\|P02790\|HEMO_HUMAN |
| Hexokinase-1 OS = Homo sapiens GN = HK1 PE = 1 SV = 3 | sp\|P19367\|HXK1_HUMAN |
| Histone H4 OS = Homo sapiens GN = HIST1H4A PE = 1 SV = 2 | sp\|P62805\|H4_HUMAN |
| Hyaluronan-binding protein 2 OS = Homo sapiens GN = HABP2 PE = 1 SV = 1 | sp\|Q14520\|HABP2_HUMAN |
| Ig heavy chain V-I region EU OS = Homo sapiens PE = 1 SV = 1 | sp\|P01742\|HV101_HUMAN |
| Ig heavy chain V-I region HG3 OS = Homo sapiens PE = 3 SV = 1 | sp\|P01743\|HV102_HUMAN |
| Ig heavy chain V-I region V35 OS = Homo sapiens PE = 1 SV = 1 | sp\|P23083\|HV103_HUMAN |
| Ig heavy chain V-I region WOL OS = Homo sapiens PE = 1 SV = 1 | sp\|P01760\|HV105_HUMAN |
| Ig heavy chain V-III region 23 OS = Homo sapiens GN = IGHV3-23 PE = 1 SV = 2 | sp\|P01764\|HV303_HUMAN |
| Ig heavy chain V-III region BUT OS = Homo sapiens PE = 1 SV = 1 | sp\|P01767\|HV306_HUMAN |
| Ig heavy chain V-III region CAM OS = Homo sapiens PE = 1 SV = 1 | sp\|P01768\|HV307_HUMAN |
| Ig heavy chain V-III region GAL OS = Homo sapiens PE = 1 SV = 1 | sp\|P01781\|HV320_HUMAN |
| Ig kappa chain C region OS = Homo sapiens GN = IGKC PE = 1 SV = 1 | sp\|P01834\|IGKC_HUMAN |
| Ig kappa chain V-I region DEE OS = Homo sapiens PE = 1 SV = 1 | sp\|P01597\|KV105_HUMAN |
| Ig kappa chain V-I region Mev OS = Homo sapiens PE = 1 SV = 1 | sp\|P01612\|KV120_HUMAN |
| Ig kappa chain V-I region WEA OS = Homo sapiens PE = 1 SV = 1 | sp\|P01610\|KV118_HUMAN |
| Ig kappa chain V-II region RPMI 6410 OS = Homo sapiens PE = 4 SV = 1 | sp\|P06310\|KV206_HUMAN |
| Ig kappa chain V-III region HAH OS = Homo sapiens PE = 2 SV = 1 | sp\|P18135\|KV312_HUMAN |
| Ig kappa chain V-III region VG (Fragment) OS = Homo sapiens PE = 1 SV = 1 | sp\|P04433\|KV309_HUMAN |
| Ig lambda chain V-I region VOR OS = Homo sapiens PE = 1 SV = 1 | sp\|P01699\|LV101_HUMAN |
| Ig lambda chain V-III region LOI OS = Homo sapiens PE = 1 SV = 1 | sp\|P80748\|LV302_HUMAN |
| Ig lambda chain V-IV region Hil OS = Homo sapiens PE = 1 SV = 1 | sp\|P01717\|LV403_HUMAN |
| IgGFc-binding protein OS = Homo sapiens GN = FCGBP PE = 1 SV = 3 | sp\|Q9Y6R7\|FCGBP_HUMAN |
| Insulin-like growth factor-binding protein complex acid labile subunit | sp\|P35858\|ALS_HUMAN |
| OS = Homo sapiens GN = IGFALS PE = 1 SV = 1 | |
| Integrin alpha-IIb OS = Homo sapiens GN = ITGA2B PE = 1 SV = 3 | sp\|P08514\|ITA2B_HUMAN |
| Integrin beta-3 OS = Homo sapiens GN = ITGB3 PE = 1 SV = 2 | sp\|P05106\|ITB3_HUMAN |
| Inter-alpha-trypsin inhibitor heavy chain H1 OS = Homo sapiens GN = ITIH1 | sp\|P19827\|IT1H1_HUMAN |
| PE = 1 SV = 3 | |
| Inter-alpha-trypsin inhibitor heavy chain H2 OS = Homo sapiens GN = ITIH2 | sp\|P19823\|IT1H2_HUMAN |
| PE = 1 SV = 2 | |
| Inter-alpha-trypsin inhibitor heavy chain H3 OS = Homo sapiens GN = ITIH3 | sp\|Q06033\|IT1H3_HUMAN |
| PE = 1 SV = 2 | |
| Inter-alpha-trypsin inhibitor heavy chain H4 OS = Homo sapiens GN = ITIH4 | sp\|Q14624\|IT1H4_HUMAN |
| PE = 1 SV = 4 | |
| Isocitrate dehydrogenase [NADP], mitochondrial OS = Homo sapiens | sp\|P48735\|IDHP_HUMAN |
| GN = IDH2 PE = 1 SV = 2 | |
| Junction plakoglobin OS = Homo sapiens GN = JUP PE = 1 SV = 3 | sp\|P14923\|PLAK_HUMAN |
| Kallistatin OS = Homo sapiens GN = SERPINA4 PE = 1 SV = 3 | sp\|P29622\|KAIN_HUMAN |
| Keratin, type II cytoskeletal 1 OS = Homo sapiens GN = KRT1 PE = 1 SV = 6 | sp\|P04264\|K2C1_HUMAN |
| Keratin, type II cytoskeletal 2 epidermal OS = Homo sapiens GN = KRT2 PE = 1 | sp\|P35908\|K22E_HUMAN |
| SV = 2 | |
| Keratin, type II cytoskeletal 78 OS = Homo sapiens GN = KRT78 PE = 2 SV = 2 | sp\|Q8N1N4\|K2C78_HUMAN |
| Keratinocyte proline-rich protein OS = Homo sapiens GN = KPRP PE = 1 SV = 1 | sp\|Q5T749\|KPRP_HUMAN |
| Kininogen-1 OS = Homo sapiens GN = KNG1 PE = 1 SV = 2 | sp\|P01042\|KNG1_HUMAN |
| Lipopolysaccharide-binding protein OS = Homo sapiens GN = LBP PE = 1 SV = 3 | sp\|P18428\|LBP_HUMAN |
| L-lactate dehydrogenase A chain OS = Homo sapiens GN = LDHA PE = 1 SV = 2 | sp\|P00338\|LDHA_HUMAN |
| Lysozyme C OS = Homo sapiens GN = LYZ PE = 1 SV = 1 | sp\|P61626\|LYSC_HUMAN |
| Multimerin-1 OS = Homo sapiens GN = MMRN1 PE = 1 SV = 3 | sp\|Q13201\|MMRN1_HUMAN |
| Myosin-9 OS = Homo sapiens GN = MYH9 PE = 1 SV = 4 | sp\|P35579\|MYH9_HUMAN |
| N-acetylmuramoyl-L-alanine amidase OS = Homo sapiens GN = PGLYRP2 PE = 1 | sp\|Q96PD5\|PGRP2_HUMAN |
| SV = 1 | |
| Peptidyl-prolyl cis-trans isomerase B OS = Homo sapiens GN = PPIB PE = 1 SV = 2 | sp\|P23284\|PP1B_HUMAN |
| Peroxiredoxin-2 OS = Homo sapiens GN = PRDX2 PE = 1 SV = 5 | sp\|P32119\|PRDX2_HUMAN |
| Pigment epithelium-derived factor OS = Homo sapiens GN = SERPINF1 PE = 1 | sp\|P36955\|PEDF_HUMAN |
| SV = 4 | |
| Plasma kallikrein OS = Homo sapiens GN = KLKB1 PE = 1 SV = 1 | sp\|P03952\|KLKB1_HUMAN |
| Plasma protease Cl inhibitor OS = Homo sapiens GN = SERPING1 PE = 1 SV = 2 | sp\|P05155\|IC1_HUMAN |
| Plasma serine protease inhibitor OS = Homo sapiens GN = SERPINA5 PE = 1 | sp\|P05154\|IPSP_HUMAN |
| SV = 3 | |
| Platelet glycoprotein Ib alpha chain OS = Homo sapiens GN = GP1BA PE = 1 | sp\|P07359\|GP1BA_HUMAN |
| SV = 2 | |
| Platelet glycoprotein Ib beta chain OS = Homo sapiens GN = GP1BB PE = 1 SV = 1 | sp\|P13224\|GP1BB_HUMAN |
| Pleckstrin OS = Homo sapiens GN = PLEK PE = 1 SV = 3 | sp\|P08567\|PLEK_HUMAN |
| Profilin-1 OS = Homo sapiens GN = PFN1 PE = 1 SV = 2 | sp\|P07737\|PROF1_HUMAN |
| Protein AMBP OS = Homo sapiens GN = AMBP PE = 1 SV = 1 | sp\|P02760\|AMBP_HUMAN |
| Protein broad-minded OS = Homo sapiens GN = TBC1D32 PE = 2 SV = 4 | sp\|Q96NH3\|BROMI_HUMAN |
| Protein disulfide-isomerase A3 OS = Homo sapiens GN = PDIA3 PE = 1 SV = 4 | sp\|P30101\|PDIA3_HUMAN |
| Protein S100-A8 OS = Homo sapiens GN = S100A8 PE = 1 SV = 1 | sp\|P05109\|S10A8_HUMAN |
| Prothrombin OS = Homo sapiens GN = F2 PE = 1 SV = 2 | sp\|P00734\|THRB_HUMAN |
| Purine nucleoside phosphorylase OS = Homo sapiens GN = PNP PE = 1 SV = 2 | sp\|P00491\|PNPH_HUMAN |
| Pyruvate kinase PKM OS = Homo sapiens GN = PKM PE = 1 SV = 4 | sp\|P14618\|KPYM_HUMAN |
| Ras-related protein Rab-10 OS = Homo sapiens GN = RAB10 PE = 1 SV = 1 | sp\|P61026\|RAB10_HUMAN |
| Ras-related protein Rap-1b OS = Homo sapiens GN = RAP1B PE = 1 SV = 1 | sp\|P61224\|RAP1B_HUMAN |
| Reticulon-4 OS = Homo sapiens GN = RTN4 PE = 1 SV = 2 | sp\|Q9NQC3\|RTN4_HUMAN |
| Retinol-binding protein 4 OS = Homo sapiens GN = RBP4 PE = 1 SV = 3 | sp\|P02753\|RET4_HUMAN |
| Sarcoplasmic/endoplasmic reticulum calcium ATPase 3 OS = Homo sapiens | sp\|Q93084\|AT2A3_HUMAN |
| GN = ATP2A3 PE = 1 SV = 2 | |
| Serum paraoxonase/arylesterase 1 OS = Homo sapiens GN = PON1 PE = 1 SV = 3 | sp\|P27169\|PON1_HUMAN |
| Skin-specific protein 32 OS = Homo sapiens GN = XP32 PE = 1 SV = 1 | sp\|Q5T750\|XP32_HUMAN |
| Small proline-rich protein 2D OS = Homo sapiens GN = SPRR2D PE = 2 SV = 2 | sp\|P22532\|SPR2D_HUMAN |
| Talin-1 OS = Homo sapiens GN = TLN1 PE = 1 SV = 3 | sp\|Q9Y490\|TLN1_HUMAN |
| Telomere length regulation protein TEL2 homolog OS = Homo sapiens | sp\|Q9Y4R8\|TELO2_HUMAN |
| GN = TELO2 PE = 1 SV = 2 | |
| Thrombospondin-1 OS = Homo sapiens GN = THBS1 PE = 1 SV = 2 | sp\|P07996\|TSP1_HUMAN |
| Thyroxine-binding globulin OS = Homo sapiens GN = SERPINA7 PE = 1 SV = 2 | sp\|P05543\|THBG_HUMAN |
| Tublin alpha-1B chain OS = Homo sapiens GN = TUBA1B PE = 1 SV = 1 | sp\|P68363\|TBA1B_HUMAN |
| Tubulin beta chain OS = Homo sapiens GN = TUBB PE = 1 SV = 2 | sp\|P07437\|TBB5_HUMAN |
| Tubulin beta-1 chain OS = Homo sapiens GN = TUBB1 PE = 1 SV = 1 | sp\|Q9H4B7\|TBB1_HUMAN |
| Tubulin beta-2A chain OS = Homo sapiens GN = TUBB2A PE = 1 SV = 1 | sp\|Q13885\|TBB2A_HUMAN |
| Vinculin OS = Homo sapiens GN = VCL PE = 1 SV = 4 | sp\|P18206\|VINC_HUMAN |
| Voltage-dependent anion-selective channel protein 3 OS = Homo sapiens | sp\|Q9Y277\|VDAC3_HUMAN |
| GN = VDAC3 PE = 1 SV = 1 | |
| von Willebrand factor OS = Homo sapiens GN = VWF PE = 1 SV = 4 | sp\|P04275\|VWF_HUMAN |
| WD repeat-containing protein 1 OS = Homo sapiens GN = WDR1 PE = 1 SV = 4 | sp\|O75083\|WDR1_HUMAN |
| Zinc-alpha-2-glycoprotein OS = Homo sapiens GN = AZGP1 PE = 1 SV = 2 | sp\|P25311\|ZA2G_HUMAN |

Table 4 provides a profile of abnormalities in pathways and glycosylated proteins in a gastric cancer male patient. This profile, including any of protein biomarkers and pathways, can be used for diagnosing, monitoring and treating a gastric cancer patient by the present methods.

TABLE 4
A profile of abnormalities in pathways and glycosylated proteins in a gastric
cancer male patient.

| | | | Jaccard |
|---|---|---|---|
| Pathway | Glycosylated Proteins | p-value | similarity |
| Complement Activation in Macular | CFI, C1QA, C3, C4B, C1QB, | 5.30067E−16 | 7.58294E−2 |
| Degeneration | C2, C1S, C5, C8A, C6, C8B, | | |
| | C7, CRP, CLU, CFHR1, | | |
| | CFH | | |
| Complement Classical Pathway | C1QA, C3, C4B, C1QB, C2, | 3.59745E−12 | 6.25000E−2 |
| | C1S, C5, C8A, C6, C8B, C7 | | |
| Complement Cascade Activation by | C1QA, C3, C4B, C2, C1S, | 3.59689E−11 | 5.79710E−2 |
| Pentraxins | C5, C8A, C6, C8B, C7, CRP, | | |
| | CFH | | |
| Complement Activation by Lectin | C3, C4B, C2, C5, C8A, C6, | 1.19784E−8 | 4.70588E−2 |
| | C8B, C7 | | |
| Complement Alternative Pathway | CFI, C3, C5, C8A, C6, C8B, | 2.84784E−7 | 4.16667E−2 |
| | C7, CFH | | |
| Coagulation Cascade | KLKB1, KNG1, SERPINC1, | 3.21514E−6 | 3.68664E−2 |
| | F2, F5, F10, F12, F13A1 | | |
| Glycolysis | HK1, GAPDH, ENO1, PKM, | 2.40683E−5 | 3.06748E−2 |
| | ALDOA | | |
| Scavenger Receptors in Platelet | GP1BA, APOB, APOE, | 1.54933E−4 | 2.87081E−2 |
| Activation | ITGA2B, ITGB3, GP1BB | | |
| Plasmin Effects in Inflammation | A2M, KLKB1, SERPING1, | 2.53755E−4 | 2.58398E−2 |
| | C1QA, KNG1, C1QB, C1S, | | |
| | ENO1, SERPINF2, F12 | | |
| Focal Junction Assembly | ACTN1, FLNA, TLN1, VCL, | 4.05858E−4 | 2.66667E−2 |
| | CAPN1, ITGB3 | | |

Table 5A discloses glycoproteins differentially expressed in plasma of pancreatic adenocarcinoma female patients. These glycoproteins can be used for diagnosing, monitoring and treating a pancreatic adenocarcinoma patient by the present methods. In these methods, glycoproteins of Table 5A are used as a set of biomarkers indicative of pancreatic adenocarcinoma cancer.

TABLE 5A
Glycoproteins differentially expressed in plasma of pancreatic
adenocarcinoma cancer (PADC) female patients

| Identified Proteins | Accession Number |
|---|---|
| Actin, cytoplasmic 1 OS = Homo sapiens GN = ACTB PE = 1 SV = 1 | sp\|P60709\|ACTB_HUMAN |
| Alpha-1-acid glycoprotein 1 OS = Homo sapiens GN = ORM1 PE = 1 SV = 1 | sp\|P02763\|A1AG1_HUMAN |
| Alpha-1-antichymotrypsin OS = Homo sapiens GN = SERPINA3 PE = 1 | sp\|P01011\|AACT_HUMAN |
| SV = 2 | |
| Alpha-1-antitrypsin OS = Homo sapiens GN = SERPINA1 PE = 1 SV = 3 | sp\|P01009\|A1AT_HUMAN |
| Alpha-1B-glycoprotein OS = Homo sapiens GN = A1BG PE = 1 SV = 4 | sp\|P04217\|A1BG_HUMAN |
| Alpha-2-antiplasmin OS = Homo sapiens GN = SERPINF2 PE = 1 SV = 3 | sp\|P08697\|A2AP_HUMAN |
| Angiotensinogen OS = Homo sapiens GN = AGT PE = 1 SV = 1 | sp\|P01019\|ANGT_HUMAN |
| Apolipoprotein A-I OS = Homo sapiens GN = AP0A1 PE = 1 SV = 1 | sp\|P02647\|APOA1_HUMAN |
| Apolipoprotein E OS = Homo sapiens GN = APOE PE = 1 SV = 1 | sp\|P02649\|APOE_HUMAN |
| Beta-2-glycoprotein 1 OS = Homo sapiens GN = APOH PE = 1 SV = 3 | sp\|P02749\|APOH_HUMAN |
| C4b-binding protein alpha chain OS = Homo sapiens GN = C4BPA PE = 1 | sp\|P04003\|C4BPA_HUMAN |
| SV = 2 | |
| Carboxypeptidase B2 OS = Homo sapiens GN = CPB2 PE = 1 SV = 2 | sp\|Q96IY4\|CBPB2_HUMAN |
| Carboxypeptidase N catalytic chain OS = Homo sapiens GN = CPN1 | sp\|P15169\|CBPN_HUMAN |
| PE = 1 SV = 1 | |
| Carboxypeptidase N subunit 2 OS = Homo sapiens GN = CPN2 PE = 1 | sp\|P22792\|CPN2_HUMAN |
| SV = 3 | |
| CD5 antigen-like OS = Homo sapiens GN = CD5L PE = 1 SV = 1 | sp\|O43866\|CD5L_HUMAN |
| Ceruloplasmin OS = Homo sapiens GN = CP PE = 1 SV = 1 | sp\|P00450\|CERU_HUMAN |
| Clusterin OS = Homo sapiens GN = CLU PE = 1 SV = 1 | sp\|P10909\|CLUS_HUMAN |
| Complement C1s subcomponent OS = Homo sapiens GN = C1S PE = 1 | sp\|P09871\|C1S_HUMAN |
| SV = 1 | |
| Complement C5 OS = Homo sapiens GN = C5 PE = 1 SV = 4 | sp\|P01031\|CO5_HUMAN |
| Complement component C7 OS = Homo sapiens GN = C7 PE = 1 SV = 2 | sp\|P10643\|CO7_HUMAN |
| Complement component C9 OS = Homo sapiens GN = C9 PE = 1 SV = 2 | sp\|P02748\|CO9_HUMAN |
| Complement factor I OS = Homo sapiens GN = CFI PE = 1 SV = 2 | sp\|P05156\|CFAI_HUMAN |
| Corticosteroid-binding globulin OS = Homo sapiens GN = SERPINA6 | sp\|P08185\|CBG_HUMAN |
| PE = 1 SV = 1 | |
| C-reactive protein OS = Homo sapiens GN = CRP PE = 1 SV = 1 | sp\|P02741\|CRP_HUMAN |
| Desmoplakin OS = Homo sapiens GN = DSP PE = 1 SV = 3 | sp\|P15924\|DESP_HUMAN |
| EGF-containing fibulin-like extracellular matrix protein 1 OS = Homo | sp\|Q12805\|FBLN3_HUMAN |
| sapiens GN = EFEMP1 PE = 1 SV = 2 | |
| Fibrinogen alpha chain OS = Homo sapiens GN = FGA PE = 1 SV = 2 | sp\|P02671\|FIBA_HUMAN |
| Fibronectin OS = Homo sapiens GN = FN1 PE = 1 SV = 4 | sp\|P02751\|FINC_HUMAN |
| Fibulin-1 OS = Homo sapiens GN = FBLN1 PE = 1 SV = 4 | sp\|P23142\|FBLN1_HUMAN |
| Ficolin-3 OS = Homo sapiens GN = FCN3 PE = 1 SV = 2 | sp\|O75636\|FCN3_HUMAN |
| Gelsolin OS = Homo sapiens GN = GSN PE = 1 SV = 1 | sp\|P06396\|GELS_HUMAN |
| Haptoglobin OS = Homo sapiens GN = HP PE = 1 SV = 1 | sp\|P00738\|HPT_HUMAN |
| Hemoglobin subunit alpha OS = Homo sapiens GN = HBA1 PE = 1 SV = 2 | sp\|P69905\|HBA_HUMAN |
| Hemoglobin subunit beta OS = Homo sapiens GN = HBB PE = 1 SV = 2 | sp\|P68871\|HBB_HUMAN |
| Heparin cofactor 2 OS = Homo sapiens GN = SERPIND1 PE = 1 SV = 3 | sp\|P05546\|HEP2_HUMAN |
| Histidine-rich glycoprotein OS = Homo sapiens GN = HRG PE = 1 SV = 1 | sp\|P04196\|HRG_HUMAN |
| Hornerin OS = Homo sapiens GN = HRNR PE = 1 SV = 2 | sp\|Q86YZ3\|HORN_HUMAN |
| Ig alpha-2 chain C region OS = Homo sapiens GN = IGHA2 PE = 1 SV = 3 | sp\|P01877\|IGHA2_HUMAN |
| Ig gamma-4 chain C region OS = Homo sapiens GN = IGHG4 PE = 1 SV = 1 | sp\|P01861\|IGHG4_HUMAN |
| Ig heavy chain V-II region WAH OS = Homo sapiens PE = 1 SV = 1 | sp\|P01824\|HV206_HUMAN |
| Ig kappa chain V-I region DEE OS = Homo sapiens PE = 1 SV = 1 | sp\|P01597\|KV105_HUMAN |
| Ig kappa chain V-I region WEA OS = Homo sapiens PE = 1 SV = 1 | sp\|P01610\|KV118_HUMAN |
| Ig kappa chain V-II region FR OS = Homo sapiens PE = 1 SV = 1 | sp\|P01615\|KV202_HUMAN |
| Ig kappa chain V-II region TEW OS = Homo sapiens PE = 1 SV = 1 | sp\|P01617\|KV204_HUMAN |
| Ig kappa chain V-IV region Len OS = Homo sapiens PE = 1 SV = 2 | sp\|P01625\|KV402_HUMAN |
| Ig lambda chain V-I region NEW OS = Homo sapiens PE = 1 SV = 1 | sp\|P01701\|LV103_HUMAN |
| Ig lambda chain V-III region LOI OS = Homo sapiens PE = 1 SV = 1 | sp\|P80748\|LV302_HUMAN |
| Ig mu chain C region OS = Homo sapiens GN = IGHM PE = 1 SV = 3 | sp\|P01871\|IGHM_HUMAN |
| Immunoglobulin J chain OS = Homo sapiens GN = IGJ PE = 1 SV = 4 | sp\|P01591\|IGJ_HUMAN |
| Inter-alpha-trypsin inhibitor heavy chain H2 OS = Homo sapiens | sp\|P19823\|IT1H2_HUMAN |
| GN = ITIH2 PE = 1 SV = 2 | |
| Inter-alpha-trypsin inhibitor heavy chain H3 OS = Homo sapiens | sp\|Q06033\|IT1H3_HUMAN |
| GN = ITIH3 PE = 1 SV = 2 | |
| Inter-alpha-trypsin inhibitor heavy chain H4 OS = Homo sapiens | sp\|Q14624\|IT1H4_HUMAN |
| GN = ITIH4 PE = 1 SV = 4 | |
| Junction plakoglobin OS = Homo sapiens GN = JUP PE = 1 SV = 3 | sp\|P14923\|PLAK_HUMAN |
| Keratin, type I cytoskeletal 9 OS = Homo sapiens GN = KRT9 PE = 1 SV = 3 | sp\|P35527\|K1C9_HUMAN |
| Keratin, type II cytoskeletal 1 OS = Homo sapiens GN = KRT1 PE = 1 SV = 6 | sp\|P04264\|K2C1_HUMAN |
| Kininogen-1 OS = Homo sapiens GN = KNG1 PE = 1 SV = 2 | sp\|P01042\|KNG1_HUMAN |
| Leucine-rich alpha-2-glycoprotein OS = Homo sapiens GN = LRG1 PE = 1 | sp\|P02750\|A2GL_HUMAN |
| SV = 2 | |
| Peptidyl-prolyl cis-trans isomerase FKBP1A OS = Homo sapiens | sp\|P62942\|FKB1A_HUMAN |
| GN = FKBP1A PE = 1 SV = 2 | |
| Peroxiredoxin-2 OS = Homo sapiens GN = PRDX2 PE = 1 SV = 5 | sp\|P32119\|PRDX2_HUMAN |
| Phosphatidylinositol-glycan-specific phospholipase D OS = Homo | sp\|P80108\|PHLD_HUMAN |
| sapiens GN = GPLD1 PE = 1 SV = 3 | |
| Pigment epithelium-derived factor OS = Homo sapiens GN = SERPINF1 | sp\|P36955\|PEDF_HUMAN |
| PE = 1 SV = 4 | |
| Plasma kallikrein OS = Homo sapiens GN = KLKB1 PE = 1 SV = 1 | sp\|P03952\|KLKB1_HUMAN |
| Plasma protease C1 inhibitor OS = Homo sapiens GN = SERPING1 PE = 1 | sp\|P05155\|IC1_HUMAN |
| SV = 2 | |
| Plasma serine protease inhibitor OS = Homo sapiens GN = SERPINA5 | sp\|P05154\|IPSP_HUMAN |
| PE = 1 SV = 3 | |
| Pregnancy zone protein OS = Homo sapiens GN = PZP PE = 1 SV = 4 | sp\|P20742\|PZP_HUMAN |
| Profilin-1 OS = Homo sapiens GN = PFN1 PE = 1 SV = 2 | sp\|P07737\|PROF1_HUMAN |
| Protein AMBP OS = Homo sapiens GN = AMBP PE = 1 SV = 1 | sp\|P02760\|AMBP_HUMAN |
| Protein S100-A8 OS = Homo sapiens GN = S100A8 PE = 1 SV = 1 | sp\|P05109\|S10A8_HUMAN |
| Prothrombin OS = Homo sapiens GN = F2 PE = 1 SV = 2 | sp\|P00734\|THRB_HUMAN |
| Serotransferrin OS = Homo sapiens GN = TF PE = 1 SV = 3 | sp\|P02787\|TRFE_HUMAN |
| Thyroxine-binding globulin OS = Homo sapiens GN = SERPINA7 PE = 1 | sp\|P05543\|THBG_HUMAN |
| SV = 2 | |
| Transthyretin OS = Homo sapiens GN = TTR PE = 1 SV = 1 | sp\|P02766\|TTHY_HUMAN |
| Vitamin K-dependent protein S OS = Homo sapiens GN = PROS1 PE = 1 | sp\|P07225\|PROS_HUMAN |
| SV = 1 | |
| Zinc-alpha-2-glycoprotein OS = Homo sapiens GN = AZGP1 PE = 1 SV = 2 | sp\|P25311\|ZA2G_HUMAN |

Table 5 provides a profile of abnormalities in pathways and abnormally glycosylated proteins in a pancreatic adenocarcinoma female patient. This profile, including any of protein biomarkers and pathways, can be used for diagnosing, monitoring and treating a pancreatic adenocarcinoma patient by the present methods.

TABLE 5
A profile of abnormalities in pathways and glycosylated proteins in a
pancreatic adenocarcinoma female patient.

| | | | Jaccard |
|---|---|---|---|
| Pathway | Glycosylated Proteins | p-value | similarity |
| Complement Activation in Macular | CFI, C1S, C5, C9, C7, | 2.82682E−7 | 4.48718E−2 |
| Degeneration | CRP, CLU | | |
| Complement Cascade Activation by | C4BPA, C1S, C5, C9, C7, | 3.20201E−6 | 4.02685E−2 |
| Pentraxins | CRP | | |
| Complement Classical Pathway | IGHM, C1S, C5, C9, C7 | 5.81054E−6 | 4.23729E−2 |
| Plasmin Effects in Inflammation | FGA, FN1, KLKB1, | 3.08961E−5 | 2.46154E−2 |
| | SERPING1, KNG1, TF, | | |
| | C1S, SERPINF2 | | |
| Coagulation Cascade | FGA, KLKB1, KNG1, | 7.58898E−5 | 3.20513E−2 |
| | PROS1, F2 | | |
| Positive Acute Phase Proteins | APOA1, APOE, | 8.37736E−5 | 1.81818E−2 |
| Synthesis | SERPINA3, CP, CRP, | | |
| | SERPINA1, FGA, FN1, | | |
| | ORM1, HP | | |
| Complement Alternative Pathway | CFI, C5, C9, C7 | 2.98648E−4 | 3.03030E−2 |
| Complement Activation by Lectin | C5, C9, C7 | 1.35026E−3 | 2.70270E−2 |
| Bradykinin Effects in Inflammation | KLKB1, KNG1, CPN1, | 1.54179E−3 | 2.36686E−2 |
| | AGT | | |
| Histidine-Rich Glycoprotein (HRG) | FGA, HRG, SERPIND1, | 4.26986E−3 | 1.97044E−2 |
| | SERPINA5 | | |

Table 6A discloses glycoproteins differentially expressed in plasma of pancreatic adenocarcinoma male patients. These glycoproteins can be used for diagnosing, monitoring and treating a pancreatic adenocarcinoma patient by the present methods. In these methods, glycoproteins of Table 6A are used as a set of biomarkers indicative of pancreatic adenocarcinoma cancer.

TABLE 6A
Glycoproteins differentially expressed in plasma of pancreatic
adenocarcinoma cancer (PADC) male patients

| Identified Proteins | Accession Number |
|---|---|
| Afamin OS = Homo sapiens GN = AFM PE = 1 SV = 1 | sp\|P43652\|AFAM_HUMAN |
| Alpha-1-acid glycoprotein 1 OS = Homo sapiens GN = ORM1 PE = 1 SV = 1 | sp\|P02763\|A1AG1_HUMAN |
| Alpha-1-antitrypsin OS = Homo sapiens GN = SERPINA1 PE = 1 SV = 3 | sp\|P01009\|A1AT_HUMAN |
| Alpha-2-macroglobulin OS = Homo sapiens GN = A2M PE = 1 SV = 3 | sp\|P01023\|A2MG_HUMAN |
| Apolipoprotein A-I OS = Homo sapiens GN = APOA1 PE = 1 SV = 1 | sp\|P02647\|APOA1_HUMAN |
| Apolipoprotein A-II OS = Homo sapiens GN = APOA2 PE = 1 SV = 1 | sp\|P02652\|APOA2_HUMAN |
| Apolipoprotein B-100 OS = Homo sapiens GN = APOB PE = 1 SV = 2 | sp\|P04114\|APOB_HUMAN |
| Apolipoprotein E OS = Homo sapiens GN = APOE PE = 1 SV = 1 | sp\|P02649\|APOE_HUMAN |
| C4b-binding protein alpha chain OS = Homo sapiens GN = C4BPA PE = 1 | sp\|P04003\|C4BPA_HUMAN |
| SV = 2 | |
| Carboxypeptidase B2 OS = Homo sapiens GN = CPB2 PE = 1 SV = 2 | sp\|Q96IY4\|CBPB2_HUMAN |
| Carboxypeptidase N catalytic chain OS = Homo sapiens GN = CPN1 PE = 1 | sp\|P15169\|CBPN_HUMAN |
| SV = 1 | |
| Carboxypeptidase N subunit 2 OS = Homo sapiens GN = CPN2 PE = 1 | sp\|P22792\|CPN2_HUMAN |
| SV = 3 | |
| Ceruloplasmin OS = Homo sapiens GN = CP PE = 1 SV = 1 | sp\|P00450\|CERU_HUMAN |
| Clusterin OS = Homo sapiens GN = CLU PE = 1 SV = 1 | sp\|P10909\|CLUS_HUMAN |
| Complement C1s subcomponent OS = Homo sapiens GN = C1S PE = 1 | sp\|P09871\|C1S_HUMAN |
| SV = 1 | |
| Complement C5 OS = Homo sapiens GN = C5 PE = 1 SV = 4 | sp\|P01031\|CO5_HUMAN |
| Complement factor B OS = Homo sapiens GN = CFB PE = 1 SV = 2 | sp\|P00751\|CFAB_HUMAN |
| C-reactive protein OS = Homo sapiens GN = CRP PE = 1 SV = 1 | sp\|P02741\|CRP_HUMAN |
| Desmoplakin OS = Homo sapiens GN = DSP PE = 1 SV = 3 | sp\|P15924\|DESP_HUMAN |
| EGF-containing fibulin-like extracellular matrix protein 1 OS = Homo | sp\|Q12805\|FBLN3_HUMAN |
| sapiens GN = EFEMP1 PE = 1 SV = 2 | |
| Fibrinogen alpha chain OS = Homo sapiens GN = FGA PE = 1 SV = 2 | sp\|P02671\|FIBA_HUMAN |
| Fibrinogen beta chain OS = Homo sapiens GN = FGB PE = 1 SV = 2 | sp\|P02675\|FIBB_HUMAN |
| Fibrinogen gamma chain OS = Homo sapiens GN = FGG PE = 1 SV = 3 | sp\|P02679\|FIBG_HUMAN |
| Fibronectin OS = Homo sapiens GN = FN1 PE = 1 SV = 4 | sp\|P02751\|FINC_HUMAN |
| Galectin-3-binding protein OS = Homo sapiens GN = LGALS3BP PE = 1 | sp\|Q08380\|LG3BP_HUMAN |
| SV = 1 | |
| Gelsolin OS = Homo sapiens GN = GSN PE = 1 SV = 1 | sp\|P06396\|GELS_HUMAN |
| Haptoglobin OS = Homo sapiens GN = HP PE = 1 SV = 1 | sp\|P00738\|HPT_HUMAN |
| Hornerin OS = Homo sapiens GN = HRNR PE = 1 SV = 2 | sp\|Q86YZ3\|HORN_HUMAN |
| Identified Proteins (228) | Accession Number |
| Ig alpha-2 chain C region OS = Homo sapiens GN = IGHA2 PE = 1 SV = 3 | sp\|P01877\|IGHA2_HUMAN |
| Ig gamma-4 chain C region OS = Homo sapiens GN = IGHG4 PE = 1 SV = 1 | sp\|P01861\|IGHG4_HUMAN |
| Ig heavy chain V-III region BUR OS = Homo sapiens PE = 1 SV = 1 | sp\|P01773\|HV312_HUMAN |
| Ig kappa chain V-I region WEA OS = Homo sapiens PE = 1 SV = 1 | sp\|P01610\|KV118_HUMAN |
| Ig kappa chain V-II region FR OS = Homo sapiens PE = 1 SV = 1 | sp\|P01615\|KV202_HUMAN |
| Ig lambda chain V-I region NEW OS = Homo sapiens PE = 1 SV = 1 | sp\|P01701\|LV103_HUMAN |
| Ig mu chain C region OS = Homo sapiens GN = IGHM PE = 1 SV = 3 | sp\|P01871\|IGHM_HUMAN |
| IgGFc-binding protein OS = Homo sapiens GN = FCGBP PE = 1 SV = 3 | sp\|Q9Y6R7\|FCGBP_HUMAN |
| Inter-alpha-trypsin inhibitor heavy chain H2 OS = Homo sapiens | sp\|P19823\|ITIH2_HUMAN |
| GN = ITIH2 PE = 1 SV = 2 | |
| Inter-alpha-trypsin inhibitor heavy chain H3 OS = Homo sapiens | sp\|Q06033\|ITIH3_HUMAN |
| GN = ITIH3 PE = 1 SV = 2 | |
| Inter-alpha-trypsin inhibitor heavy chain H4 OS = Homo sapiens | sp\|Q14624\|ITIH4_HUMAN |
| GN = ITIH4 PE = 1 SV = 4 | |
| Kallistatin OS = Homo sapiens GN = SERPINA4 PE = 1 SV = 3 | sp\|P29622\|KAIN_HUMAN |
| Keratin, type I cytoskeletal 9 OS = Homo sapiens GN = KRT9 PE = 1 SV = 3 | sp\|P35527\|K1C9_HUMAN |
| Keratin, type II cytoskeletal 1 OS = Homo sapiens GN = KRT1 PE = 1 SV = 6 | sp\|P04264\|K2C1_HUMAN |
| Keratinocyte proline-rich protein OS = Homo sapiens GN = KPRP PE = 1 | sp\|Q5T749\|KPRP_HUMAN |
| SV = 1 | |
| Kininogen-1 OS = Homo sapiens GN = KNG1 PE = 1 SV = 2 | sp\|P01042\|KNG1_HUMAN |
| Lipopolysaccharide-binding protein OS = Homo sapiens GN = LBP PE = 1 | sp\|P18428\|LBP_HUMAN |
| SV = 3 | |
| Peptidyl-prolyl cis-trans isomerase FKBP1A OS = Homo sapiens | sp\|P62942\|FKB1A_HUMAN |
| GN = FKBP1A PE = 1 SV = 2 | |
| Phosphatidylinositol-glycan-specific phospholipase D OS = Homo | sp\|P80108\|PHLD_HUMAN |
| sapiens GN = GPLD1 PE = 1 SV = 3 | |
| Polymeric immunoglobulin receptor OS = Homo sapiens GN = PIGR PE = 1 | sp\|P01833\|PIGR_HUMAN |
| SV = 4 | |
| Pregnancy zone protein OS = Homo sapiens GN = PZP PE = 1 SV = 4 | sp\|P20742\|PZP_HUMAN |
| Profilin-1 OS = Homo sapiens GN = PFN1 PE = 1 SV = 2 | sp\|P07737\|PROF1_HUMAN |
| Protein AMBP OS = Homo sapiens GN = AMBP PE = 1 SV = 1 | sp\|P02760\|AMBP_HUMAN |
| Serotransferrin OS = Homo sapiens GN = TF PE = 1 SV = 3 | sp\|P02787\|TRFE_HUMAN |
| Serum amyloid A-4 protein OS = Homo sapiens GN = SAA4 PE = 1 SV = 2 | sp\|P35542\|SAA4_HUMAN |
| Sex hormone-binding globulin OS = Homo sapiens GN = SHBG PE = 1 | sp\|P04278\|SHBG_HUMAN |
| SV = 2 | |
| Thyroxine-binding globulin OS = Homo sapiens GN = SERPINA7 PE = 1 | sp\|P05543\|THBG_HUMAN |
| SV = 2 | |
| Vitamin K-dependent protein S OS = Homo sapiens GN = PROS1 PE = 1 | sp\|P07225\|PROS_HUMAN |
| SV = 1 | |
| Zinc-alpha-2-glycoprotein OS = Homo sapiens GN = AZGP1 PE = 1 SV = 2 | sp\|P25311\|ZA2G_HUMAN |

Table 6 provides a profile of abnormalities in pathways and glycosylated proteins in a pancreatic adenocarcinoma male patient. This profile, including any of protein biomarkers and pathways, can be used for diagnosing, monitoring and treating a pancreatic adenocarcinoma patient by the present methods.

TABLE 6
A profile of abnormalities in pathways and glycosylated proteins in a
pancreatic adenocarcinoma male patient.

| | | | Jaccard |
|---|---|---|---|
| Pathway | Glycosylated Proteins | p-value | similarity |
| Positive Acute Phase | APOA1, APOA2, APOE, A2M, | 9.40620E−10 | 2.63653E−2 |
| Proteins Synthesis | CP, CRP, SERPINA1, LBP, | | |
| | FGA, FGB, FGG, FN1, ORM1, | | |
| | HP | | |
| TAM Receptors in Platelet | FGA, FGB, FGG, PROS1 | 2.84129E−6 | 5.26316E−2 |
| Aggregation | | | |
| Plasmin Effects in | FGA, FGB, FGG, FN1, A2M, | 4.87208E−6 | 2.58065E−2 |
| Inflammation | KNG1, TF, C1S | | |
| Complement Cascade | CFB, C4BPA, C1S, C5, CRP | 1.70620E−5 | 3.70370E−2 |
| Activation by Pentraxins | | | |
| Coagulation Cascade | FGA, FGB, FGG, KNG1, | 2.36122E−5 | 3.54610E−2 |
| | PROS1 | | |
| Complement Activation in | CFB, C1S, C5, CRP, CLU | 2.61822E−5 | 3.49650E−2 |
| Macular Degeneration | | | |
| Scavenger Receptors in | APOB, FGA, FGB, FGG | 7.22674E−5 | 3.66972E−2 |
| Platelet Aggregation | | | |
| Scavenger Receptors in | APOB, APOA1, APOA2, APOE | 2.56073E−4 | 3.03030E−2 |
| Platelet Activation | | | |
| Vascular Endothelial Cell | FGA, FGB, EGG, PROS1, | 4.45784E−4 | 2.26244E−2 |
| Activation by Blood | KNG1 | | |
| Coagulation Factors | | | |
| Lipogenesis Regulation in | APOB, APOA1, APOA2, APOE | 4.65075E−4 | 2.73973E−2 |
| Adipocytes | | | |

Table 7A discloses glycoproteins differentially expressed in plasma of liver cancer patients. These glycoproteins can be used for diagnosing, monitoring and treating a liver cancer patient by the present methods. In these methods, glycoproteins of Table 5A are used as a set of biomarkers indicative of liver cancer.

TABLE 7A
Glycoproteins differentially expressed in plasma of liver cancer patients

| Identified Proteins (739) | Accession Number |
|---|---|
| Actin, cytoplasmic 2 OS = Homo sapiens GN = ACTG1 PE = 1 SV = 1 | sp\|P63261\|ACTG_HUMAN |
| Alpha-1B-glycoprotein OS = Homo sapiens GN = A1BG PE = 1 SV = 4 | sp\|P04217\|A1BG_HUMAN |
| Apolipoprotein B-100 OS = Homo sapiens GN = APOB PE = 1 SV = 2 | sp\|P04114\|APOB_HUMAN |
| Apolipoprotein D OS = Homo sapiens GN = APOD PE = 1 SV = 1 | sp\|P05090\|APOD_HUMAN |
| Apolipoprotein E OS = Homo sapiens GN = APOE PE = 1 SV = 1 | sp\|P02649\|APOE_HUMAN |
| Apolipoprotein L1 OS = Homo sapiens GN = APOL1 PE = 1 SV = 5 | sp\|O14791\|APOL1_HUMAN |
| Apolipoprotein(a) OS = Homo sapiens GN = LPA PE = 1 SV = 1 | sp\|P08519\|APOA_HUMAN |
| ATP-binding cassette sub-family B member 9 OS = Homo sapiens GN = ABCB9 | sp\|Q9NP78\|ABCB9_HUMAN |
| PE = 1 SV = 1 | |
| Attractin OS = Homo sapiens GN = ATRN PE = 1 SV = 2 | sp\|O75882\|ATRN_HUMAN |
| C4b-binding protein alpha chain OS = Homo sapiens GN = C4BPA PE = 1 SV = 2 | sp\|P04003\|C4BPA_HUMAN |
| C4b-binding protein beta chain OS = Homo sapiens GN = C4BPB PE = 1 SV = 1 | sp\|P20851\|C4BPB_HUMAN |
| Carbonic anhydrase 1 OS = Homo sapiens GN = CA1 PE = 1 SV = 2 | sp\|P00915\|CAH1_HUMAN |
| Carboxypeptidase N catalytic chain OS = Homo sapiens GN = CPN1 PE = 1 SV = 1 | sp\|P15169\|CBPN_HUMAN |
| Cholesteryl ester transfer protein OS = Homo sapiens GN = CETP PE = 1 SV = 2 | sp\|P11597\|CETP_HUMAN |
| Coagulation factor V OS = Homo sapiens GN = F5 PE = 1 SV = 4 | sp\|P12259\|FA5_HUMAN |
| Coagulation factor X OS = Homo sapiens GN = F10 PE = 1 SV = 2 | sp\|P00742\|FA10_HUMAN |
| Coagulation factor XII OS = Homo sapiens GN = F12 PE = 1 SV = 3 | sp\|P00748\|FA12_HUMAN |
| Coagulation factor XIII A chain OS = Homo sapiens GN = F13A1 PE = 1 SV = 4 | sp\|P00488\|F13A_HUMAN |
| Complement C1q subcomponent subunit B OS = Homo sapiens GN = C1QB PE = 1 | sp\|P02746\|C1QB_HUMAN |
| SV = 3 | |
| Complement C1s subcomponent OS = Homo sapiens GN = C1S PE = 1 SV = 1 | sp\|P09871\|C1S_HUMAN |
| Complement C3 OS = Homo sapiens GN = C3 PE = 1 SV = 2 | sp\|P01024\|CO3_HUMAN |
| Complement C4-A OS = Homo sapiens GN = C4A PE = 1 SV = 2 | sp\|P0C0L4\|CO4A_HUMAN |
| Complement C4-B OS = Homo sapiens GN = C4B PE = 1 SV = 2 | sp\|P0C0L5\|CO4B_HUMAN |
| Complement C5 OS = Homo sapiens GN = C5 PE = 1 SV = 4 | sp\|P01031\|CO5_HUMAN |
| Complement component C8 beta chain OS = Homo sapiens GN = C8B PE = 1 SV = 3 | sp\|P07358\|CO8B_HUMAN |
| Complement component C9 OS = Homo sapiens GN = C9 PE = 1 SV = 2 | sp\|P02748\|CO9_HUMAN |
| Complement factor B OS = Homo sapiens GN = CFB PE = 1 SV = 2 | sp\|P00751\|CFAB_HUMAN |
| Complement factor H OS = Homo sapiens GN = CFH PE = 1 SV = 4 | sp\|P08603\|CFAH_HUMAN |
| Complement factor H-related protein 1 OS = Homo sapiens GN = CFHR1 PE = 1 | sp\|Q03591\|FHR1_HUMAN |
| SV = 2 | |
| EGF-containing fibulin-like extracellular matrix protein 1 OS = Homo sapiens | sp\|Q12805\|FBLN3_HUMAN |
| GN = EFEMP1 PE = 1 SV = 2 | |
| Fermitin family homolog 3 OS = Homo sapiens GN = FERMT3 PE = 1 SV = 1 | sp\|Q86UX7\|URP2_HUMAN |
| Fibrinogen alpha chain OS = Homo sapiens GN = FGA PE = 1 SV = 2 | sp\|P02671\|FIBA_HUMAN |
| Fibrinogen beta chain OS = Homo sapiens GN = FGB PE = 1 SV = 2 | sp\|P02675\|FIBB_HUMAN |
| Fibrinogen gamma chain OS = Homo sapiens GN = FGG PE = 1 SV = 3 | sp\|P02679\|FIBG_HUMAN |
| Fibronectin OS = Homo sapiens GN = FN1 PE = 1 SV = 4 | sp\|P02751\|FINC_HUMAN |
| Glutathione peroxidase 3 OS = Homo sapiens GN = GPX3 PE = 1 SV = 2 | sp\|P22352\|GPX3_HUMAN |
| Glyceraldehyde-3-phosphate dehydrogenase OS = Homo sapiens GN = GAPDH | sp\|P04406\|G3P_HUMAN |
| PE = 1 SV = 3 | |
| Hepatocyte growth factor-like protein OS = Homo sapiens GN = MST1 PE = 1 | sp\|P26927\|HGFL_HUMAN |
| SV = 2 | |
| Hornerin OS = Homo sapiens GN = HRNR PE = 1 SV = 2 | sp\|Q86YZ3\|HORN_HUMAN |
| Hyaluronan-binding protein 2 OS = Homo sapiens GN = HABP2 PE = 1 SV = 1 | sp\|Q14520\|HABP2_HUMAN |
| Ig alpha-2 chain C region OS = Homo sapiens GN = IGHA2 PE = 1 SV = 3 | sp\|P01877\|IGHA2_HUMAN |
| Ig delta chain C region OS = Homo sapiens GN = IGHD PE = 1 SV = 2 | sp\|P01880\|IGHD_HUMAN |
| Ig heavy chain V-I region V35 OS = Homo sapiens PE = 1 SV = 1 | sp\|P23083\|HV103_HUMAN |
| Ig heavy chain V-I region WOL OS = Homo sapiens PE = 1 SV = 1 | sp\|P01760\|HV105_HUMAN |
| Ig heavy chain V-II region MCE OS = Homo sapiens PE = 1 SV = 1 | sp\|P01817\|HV204_HUMAN |
| Ig heavy chain V-III region BUR OS = Homo sapiens PE = 1 SV = 1 | sp\|P01773\|HV312_HUMAN |
| Ig heavy chain V-III region CAM OS = Homo sapiens PE = 1 SV = 1 | sp\|P01768\|HV307_HUMAN |
| Ig heavy chain V-III region KOL OS = Homo sapiens PE = 1 SV = 1 | sp\|P01772\|HV311_HUMAN |
| Ig heavy chain V-III region NIE OS = Homo sapiens PE = 1 SV = 1 | sp\|P01770\|HV309_HUMAN |
| Ig heavy chain V-III region TIL OS = Homo sapiens PE = 1 SV = 1 | sp\|P01765\|HV304_HUMAN |
| Ig kappa chain V-I region Kue OS = Homo sapiens PE = 1 SV = 1 | sp\|P01604\|KV112_HUMAN |
| Ig kappa chain V-I region Lay OS = Homo sapiens PE = 1 SV = 1 | sp\|P01605\|KV113_HUMAN |
| Ig kappa chain V-I region Mev OS = Homo sapiens PE = 1 SV = 1 | sp\|P01612\|KV120_HUMAN |
| Ig kappa chain V-I region Scw OS = Homo sapiens PE = 1 SV = 1 | sp\|P01609\|KV117_HUMAN |
| Ig kappa chain V-I region WEA OS = Homo sapiens PE = 1 SV = 1 | sp\|P01610\|KV118_HUMAN |
| Ig kappa chain V-II region FR OS = Homo sapiens PE = 1 SV = 1 | sp\|P01615\|KV202_HUMAN |
| Ig kappa chain V-II region MIL OS = Homo sapiens PE = 1 SV = 1 | sp\|P01616\|KV203_HUMAN |
| Ig kappa chain V-II region TEW OS = Homo sapiens PE = 1 SV = 1 | sp\|P01617\|KV204_HUMAN |
| Ig kappa chain V-III region CLL OS = Homo sapiens PE = 4 SV = 2 | sp\|P04207\|KV308_HUMAN |
| Ig kappa chain V-III region IARC/BL41 OS = Homo sapiens PE = 4 SV = 1 | sp\|P06311\|KV311_HUMAN |
| Ig kappa chain V-III region SIE OS = Homo sapiens PE = 1 SV = 1 | sp\|P01620\|KV302_HUMAN |
| Ig kappa chain V-III region VG (Fragment) OS = Homo sapiens PE = 1 SV = 1 | sp\|P04433\|KV309_HUMAN |
| Ig lambda chain V-I region NEWM OS = Homo sapiens PE = 1 SV = 1 | sp\|P01703\|LV105_HUMAN |
| Ig lambda chain V-I region NIG-64 OS = Homo sapiens PE = 1 SV = 1 | sp\|P01702\|LV104_HUMAN |
| Ig lambda chain V-I region VOR OS = Homo sapiens PE = 1 SV = 1 | sp\|P01699\|LV101_HUMAN |
| Ig lambda chain V-II region TRO OS = Homo sapiens PE = 1 SV = 1 | sp\|P01707\|LV204_HUMAN |
| Ig lambda chain V-V region DEL OS = Homo sapiens PE = 1 SV = 1 | sp\|P01719\|LV501_HUMAN |
| Ig lambda chain V-VI region SUT OS = Homo sapiens PE = 1 SV = 1 | sp\|P06317\|LV603_HUMAN |
| Ig mu chain C region OS = Homo sapiens GN = IGHM PE = 1 SV = 3 | sp\|P01871\|IGHM_HUMAN |
| Ig mu heavy chain disease protein OS = Homo sapiens PE = 1 SV = 1 | sp\|P04220\|MUCB_HUMAN |
| Insulin-like growth factor-binding protein complex acid labile subunit | sp\|P35858\|ALS_HUMAN |
| OS = Homo sapiens GN = IGFALS PE = 1 SV = 1 | |
| Inter-alpha-trypsin inhibitor heavy chain H1 OS = Homo sapiens GN = ITIH1 PE = 1 | sp\|P19827\|ITIH1_HUMAN |
| SV = 3 | |
| Inter-alpha-trypsin inhibitor heavy chain H2 OS = Homo sapiens GN = ITIH2 PE = 1 | sp\|P19823\|ITIH2_HUMAN |
| SV = 2 | |
| Inter-alpha-trypsin inhibitor heavy chain H3 OS = Homo sapiens GN = ITIH3 PE = 1 | sp\|Q06033\|ITIH3_HUMAN |
| SV = 2 | |
| Inter-alpha-trypsin inhibitor heavy chain H4 OS = Homo sapiens GN = ITIH4 PE = 1 | sp\|Q14624\|ITIH4_HUMAN |
| SV = 4 | |
| Junction plakoglobin OS = Homo sapiens GN = JUP PE = 1 SV = 3 | sp\|P14923\|PLAK_HUMAN |
| Keratin, type I cytoskeletal 9 OS = Homo sapiens GN = KRT9 PE = 1 SV = 3 | sp\|P35527\|K1C9_HUMAN |
| Keratin, type II cytoskeletal 1 OS = Homo sapiens GN = KRT1 PE = 1 SV = 6 | sp\|P04264\|K2C1_HUMAN |
| Keratinocyte proline-rich protein OS = Homo sapiens GN = KPRP PE = 1 SV = 1 | sp\|Q5T749\|KPRP_HUMAN |
| Lipopolysaccharide-binding protein OS = Homo sapiens GN = LBP PE = 1 SV = 3 | sp\|P18428\|LBP_HUMAN |
| Loricrin OS = Homo sapiens GN = LOR PE = 1 SV = 2 | sp\|P23490\|LORI_HUMAN |
| Myosin-9 OS = Homo sapiens GN = MYH9 PE = 1 SV = 4 | sp\|P35579\|MYH9_HUMAN |
| Phosphatidylinositol-glycan-specific phospholipase D OS = Homo sapiens | sp\|P80108\|PHLD_HUMAN |
| GN = GPLD1 PE = 1 SV = 3 | |
| Polymeric immunoglobulin receptor OS = Homo sapiens GN = PIGR PE = 1 SV = 4 | sp\|P01833\|PIGR_HUMAN |
| Prenylcysteine oxidase 1 OS = Homo sapiens GN = PCYOX1 PE = 1 SV = 3 | sp\|Q9UHG3\|PCYOX_HUMAN |
| Profilin-1 OS = Homo sapiens GN = PFN1 PE = 1 SV = 2 | sp\|P07737\|PROF1_HUMAN |
| Properdin OS = Homo sapiens GN = CFP PE = 1 SV = 2 | sp\|P27918\|PROP_HUMAN |
| Protein AMBP OS = Homo sapiens GN = AMBP PE = 1 SV = 1 | sp\|P02760\|AMBP_HUMAN |
| Protein broad-minded OS = Homo sapiens GN = TBC1D32 PE = 2 SV = 4 | sp\|Q96NH3\|BROMI_HUMAN |
| Putative V-set and immunoglobulin domain-containing-like protein | sp\|A6NJ16\|IV4F8_HUMAN |
| IGHV4OR15-8 OS = Homo sapiens GN = IGHV4OR15-8 PE = 5 SV = 2 | |
| Ras-related protein Rap-1b OS = Homo sapiens GN = RAP1B PE = 1 SV = 1 | sp\|P61224\|RAP1B_HUMAN |
| Retinol-binding protein 4 OS = Homo sapiens GN = RBP4 PE = 1 SV = 3 | sp\|P02753\|RET4_HUMAN |
| Selenoprotein P OS = Homo sapiens GN = SEPP1 PE = 1 SV = 3 | sp\|P49908\|SEPP1_HUMAN |
| Talin-1 OS = Homo sapiens GN = TLN1 PE = 1 SV = 3 | sp\|Q9Y490\|TLN1_HUMAN |
| Telomere length regulation protein TEL2 homolog OS = Homo sapiens | sp\|Q9Y4R8\|TELO2_HUMAN |
| GN = TELO2 PE = 1 SV = 2 | |
| Tetranectin OS = Homo sapiens GN = CLEC3B PE = 1 SV = 3 | sp\|P05452\|TETN_HUMAN |
| Thrombospondin-1 OS = Homo sapiens GN = THBS1 PE = 1 SV = 2 | sp\|P07996\|TSP1_HUMAN |
| Thyroxine-binding globulin OS = Homo sapiens GN = SERPINA7 PE = 1 SV = 2 | sp\|P05543\|THBG_HUMAN |
| Transforming growth factor-beta-induced protein ig-h3 OS = Homo sapiens | sp\|Q15582\|BGH3_HUMAN |
| GN = TGFBI PE = 1 SV = 1 | |
| Tubulin alpha-1B chain OS = Homo sapiens GN = TUBA1B PE = 1 SV = 1 | sp\|P68363\|TBA1B_HUMAN |
| Vitamin D-binding protein OS = Homo sapiens GN = GC PE = 1 SV = 1 | sp\|P02774\|VTDB_HUMAN |
| Vitamin K-dependent protein C OS = Homo sapiens GN = PROC PE = 1 SV = 1 | sp\|P04070\|PROC_HUMAN |
| Vitronectin OS = Homo sapiens GN = VTN PE = 1 SV = 1 | sp\|P04004\|VTNC_HUMAN |
| Zinc-alpha-2-glycoprotein OS = Homo sapiens GN = AZGP1 PE = 1 SV = 2 | sp\|P25311\|ZA2G_HUMAN |

Table 7 provides a profile of abnormalities in pathways and glycosylated proteins in a liver cancer male patient. This profile, including any of protein biomarkers and pathways, can be used for diagnosing, monitoring and treating a liver cancer patient by the present methods.

TABLE 7
A profile of abnormalities in pathways and glycosylated
proteins in a liver cancer male patient.

| | | | Jaccard |
|---|---|---|---|
| Pathway | Glycosylated Proteins | p-value | similarity |
| Complement Activation in | CFB, C3, C4B, C1QB, CFP, | 8.32733E−15 | 7.45342E−2 |
| Macular Degeneration | C1S, C5, C9, C8B, VTN, | | |
| | CFHR1, CFH | | |
| Complement Classical | IGHM, C3, C4B, C1QB, | 1.18506E−10 | 6.40000E−2 |
| Pathway | C1S, C5, C9, C8B | | |
| Complement Cascade | CFB, C3, C4B, C4BPA, | 1.71295E−10 | 5.76923E−2 |
| Activation by Pentraxins | C1S, C5, C9, C8B, CFH | | |
| Coagulation Cascade | FGA, FGB, FGG, PROC, | 8.71371E−9 | 4.90798E−2 |
| | F5, F10, F12, F13A1 | | |
| Complement Alternative | CFB, C3, CFP, C5, C9, | 2.29775E−8 | 5.03597E−2 |
| Pathway | C8B, CFH | | |
| Complement Activation by | C3, C4B, C5, C9, C8B | 2.09678E−6 | 4.20168E−2 |
| Lectin | | | |
| Scavenger Receptors in | APOB, FGA, FGB, FGG, | 7.96085E−6 | 3.75940E−2 |
| Platelet Aggregation | TLN1 | | |
| Histidine-Rich Glycoprotein | FGA, FGB, FGG, C1QB, | 4.12511E−5 | 2.84360E−2 |
| (HRG) | THBS1, F12 | | |
| Plasmin Effects in | FGA, FGB, FGG, FN1, | 2.02105E−4 | 2.08333E−2 |
| Inflammation | C1QB, C1S, F12 | | |
| TAM Receptors in Platelet | FGA, FGB, FGG | 2.58384E−4 | 2.94118E−2 |
| Aggregation | | | |

As can be appreciated from the data in Tables 1A-7A and Tables 1-7, some of the protein markers associate specifically with a particular type of GI cancer, while other protein markers are common for several different GI cancers. As can be also appreciated from Tables 1A-7A and Tables 1-7, various pathways are affected in each of the cancers.

The term “biological pathway or pathway” is understood broadly and refers to a set of proteins (and other molecules) that act as a network to initiate, alter or terminate a biological process. Examples of biological pathways include metabolic pathways, gene-regulation pathways, and signal transduction pathways. Dozens and even hundreds of different proteins may comprise a pathway. An activation or inhibition of one protein in a pathway may trigger a chain reaction of activities in the pathway. While two different cancer patients may have a mutation in different proteins, the same pathway may be affected in both patients and lead to the same symptoms. Thus, identifying pathways affected in a cancer patient is a technical advantage of the present methods because it allows to more accurately assess the differences which cause symptoms.

According to some embodiments of the present methods, the first step is to identify proteins abnormally present in a blood or plasma sample of a patient in comparison to a healthy control, as shown in Tables 1A, 2A, 3A, 4A, 5A, 6A, and 7A. The second step is to identify pathways which are enriched (show statistically significantly overlap) with the proteins from the protein profile lists, as shown in Tables 1, 2, 3, 4, 5, 6 and 7.

Using the Fisher's exact test, nonrandom associations between two categorical variables (a pathway and a protein profile list) are determined. Each of the protein profile lists is compared to a pathway database by applying Fisher's exact test consecutively to all pathways in the database to compare the given protein profile and each of the pathways in the database. One pathway database of pathway maps that can be used for this analysis is PATHWAY STUDIO® available from Elsevier, Inc. (Nikitin et al. 2003, Bioinformatics, https://www.elsevier.com/solutions/pathway-studio-biological-research).

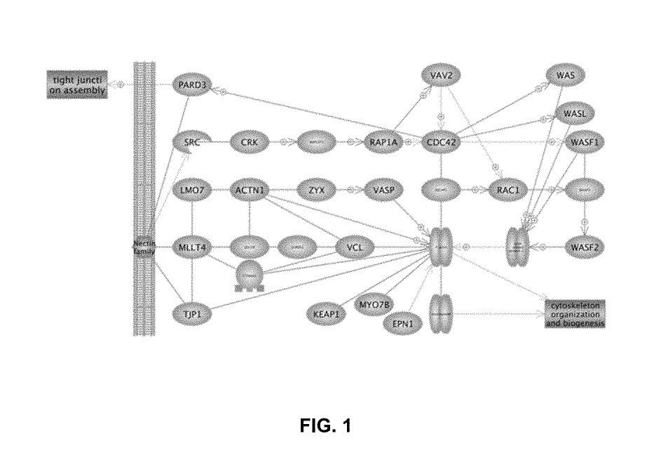

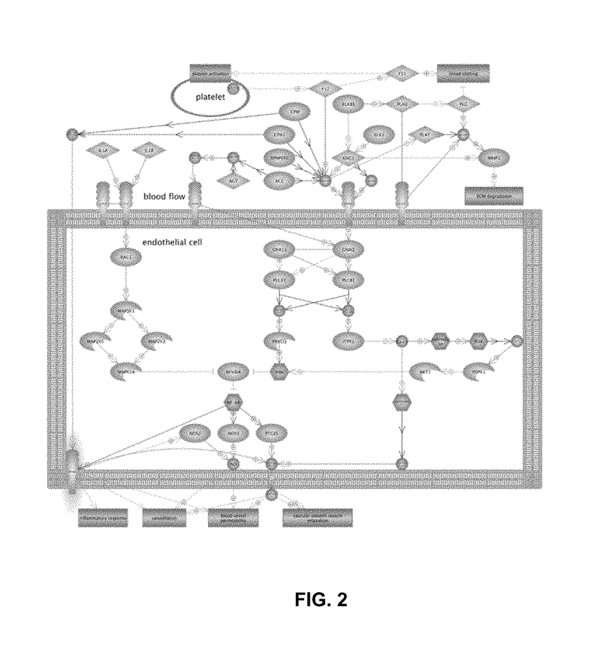

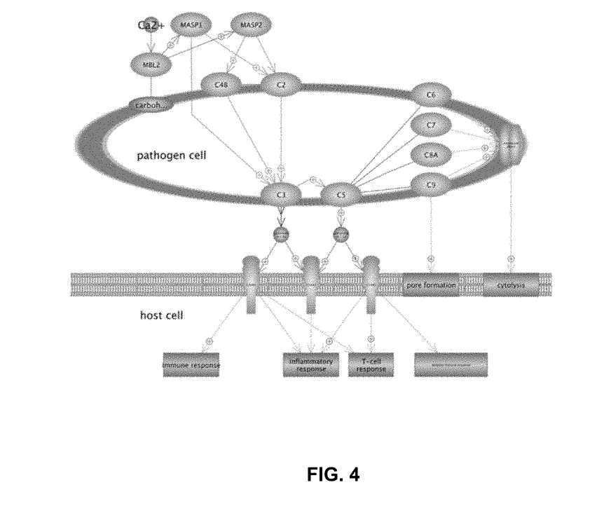

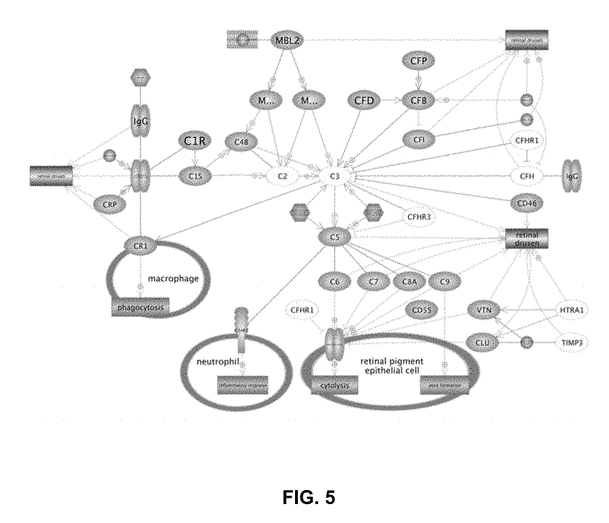

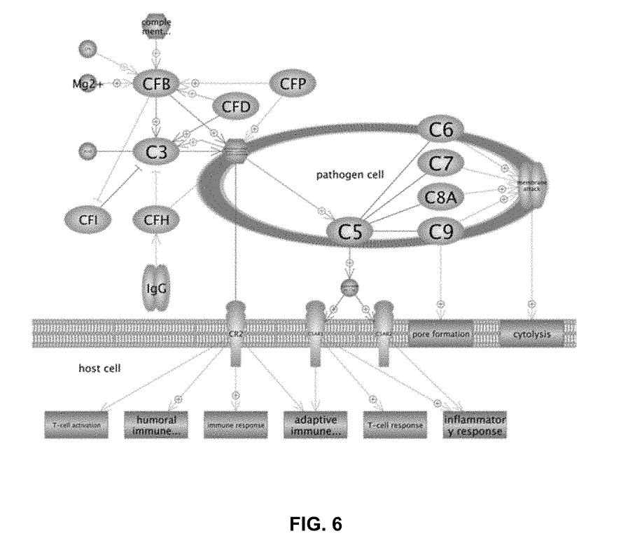

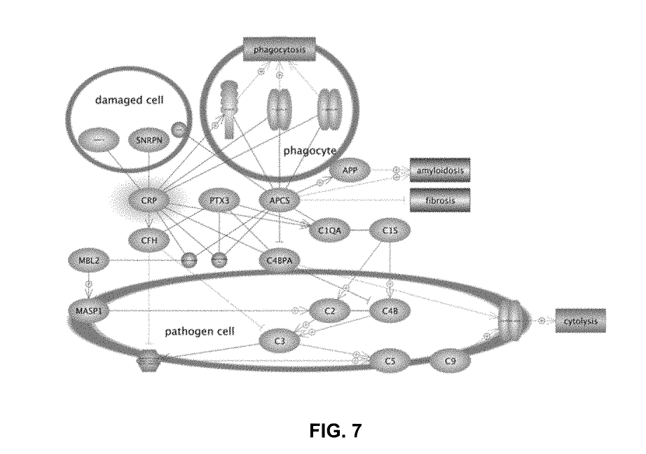

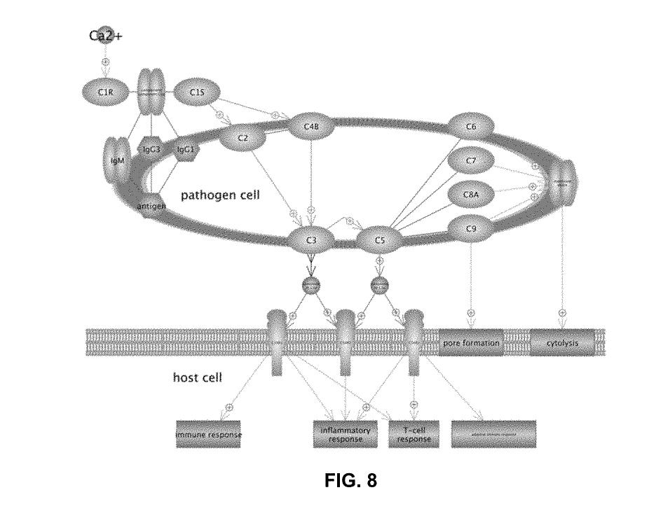

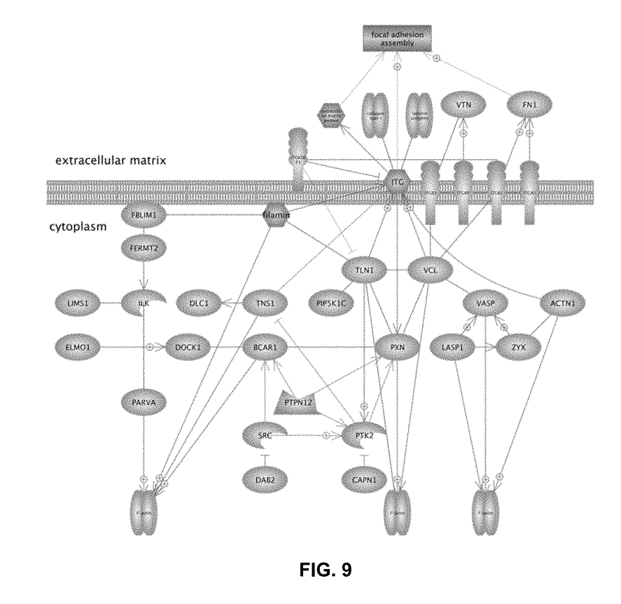

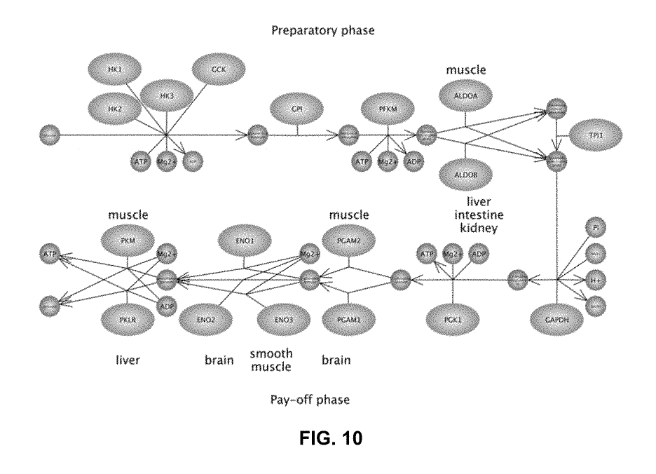

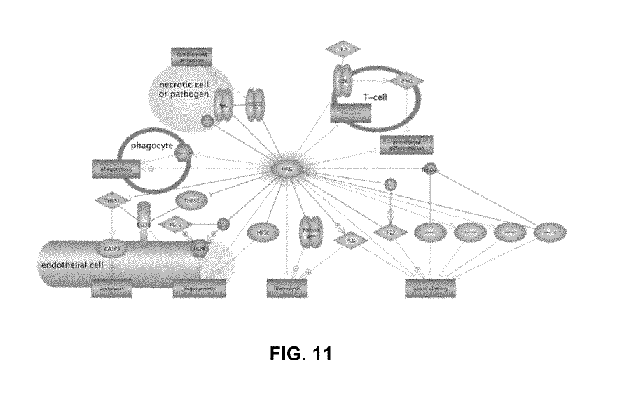

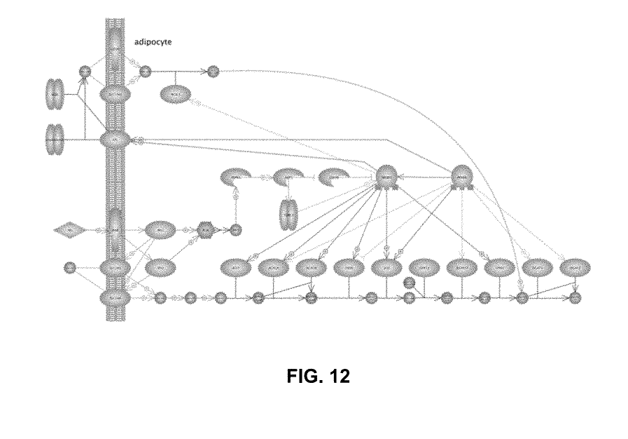

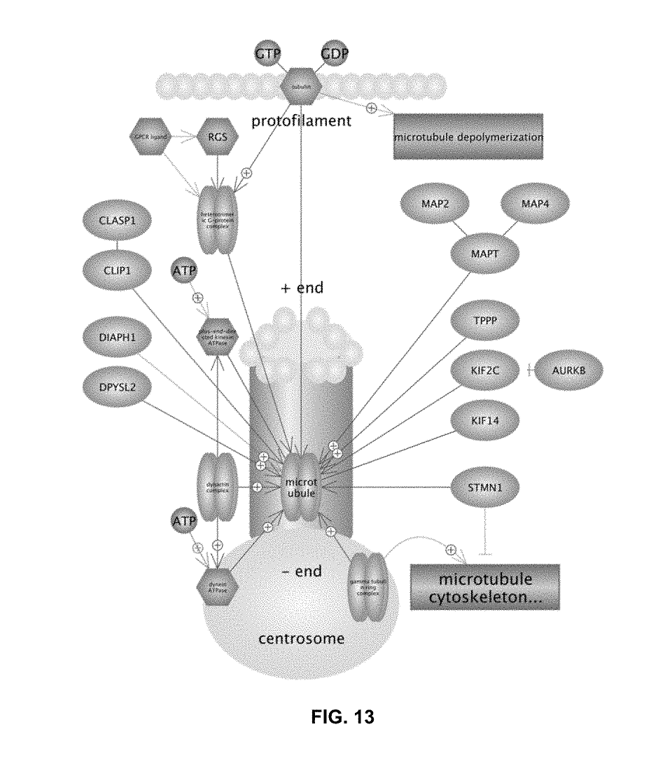

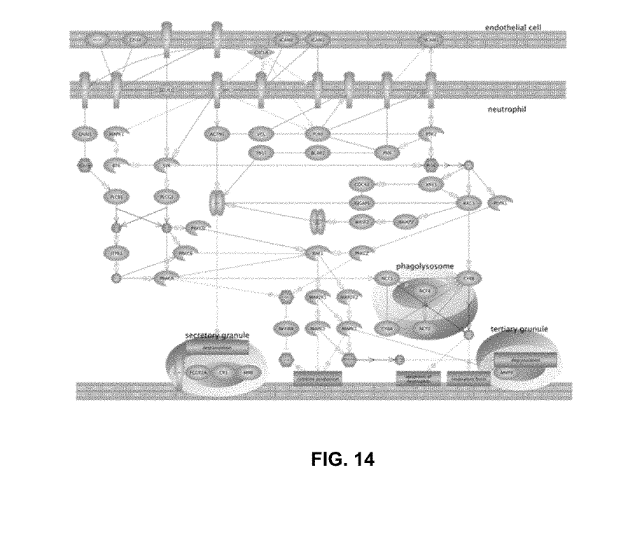

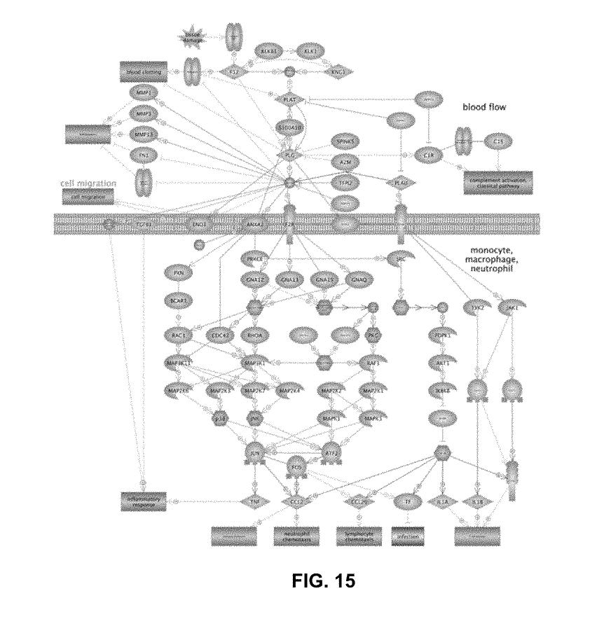

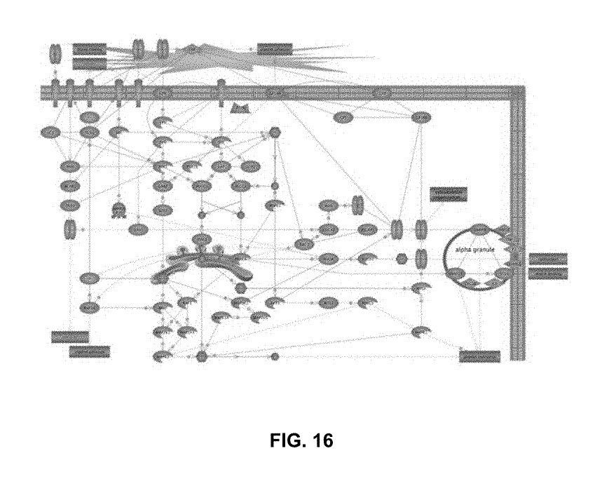

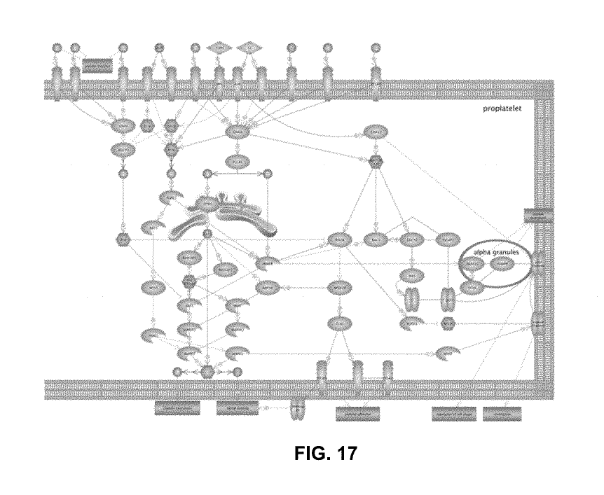

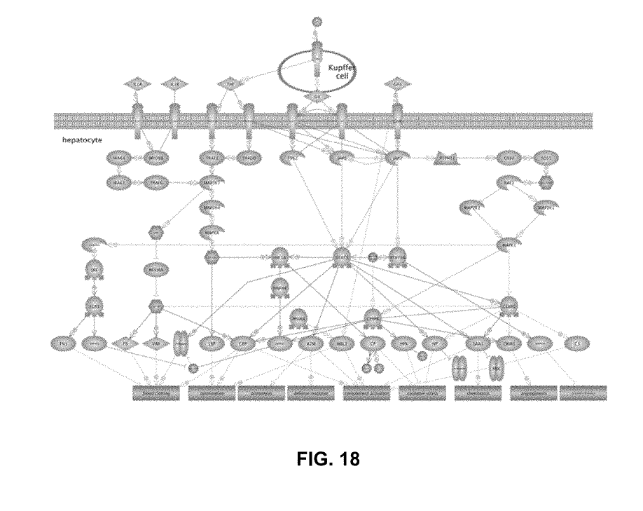

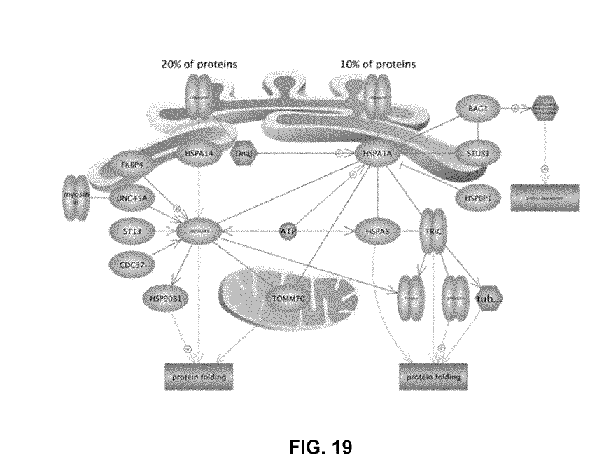

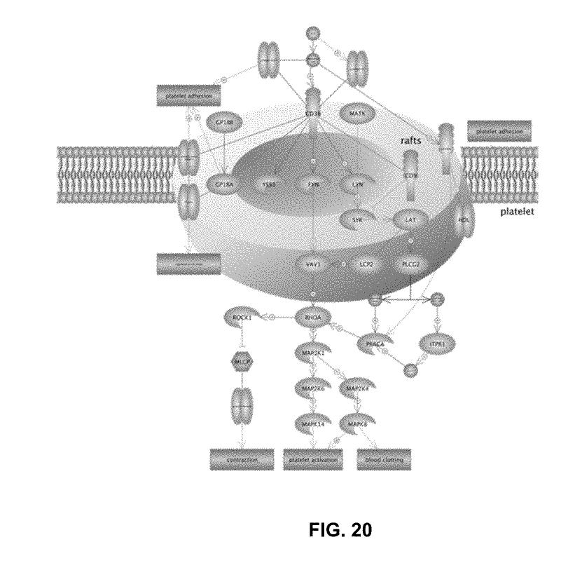

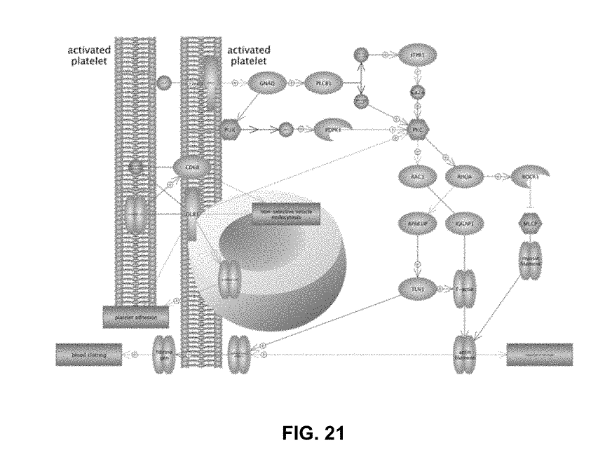

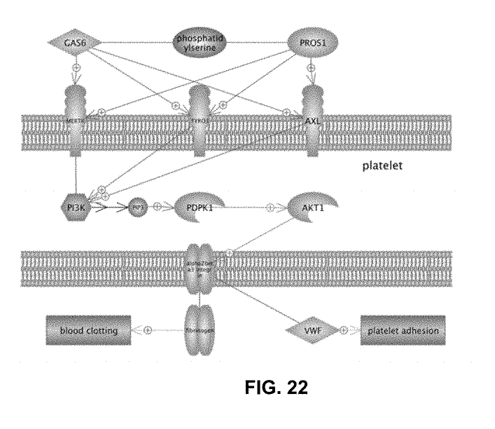

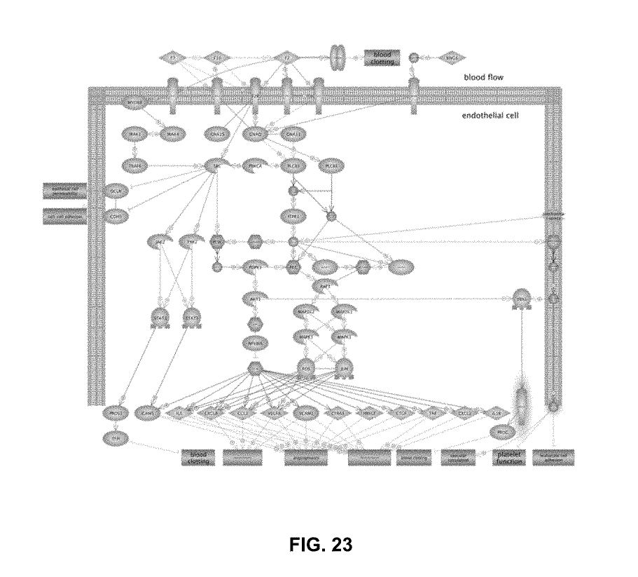

This approach was used to identify pathways for glycosylated protein biomarkers in Tables 1-7. PATHWAY STUDIO® software was also used to generate FIGS. 1-23, each of which defines a relationship between proteins in a pathway affected in at least one of GI cancers. Any of the proteins shown in FIGS. 1-23 can be included as an additional biomarker for diagnosing, monitoring and/or treating a GI cancer together with protein biomarkers from Tables 1-7.

Table 8 provides a list of pathways which can be used as a biomarker in screening, monitoring and/or treating a GI cancer. In Table 8, a pathway that can be used as a biomarker in colorectal cancer, gastric cancer, liver cancer or pancreatic cancer is identified with an XX.

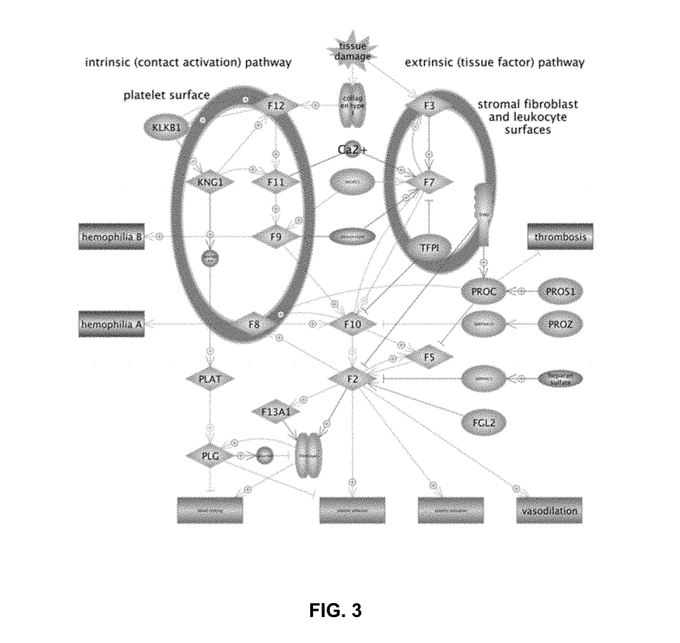

As can be seen from Table 8, some pathways, such as for example, the adherens junction assembly pathway (FIG. 1) is a specific biomarker for a gastric cancer female patient, while other pathways such as the coagulation cascade pathway (FIG. 3) is a biomarker for all GI cancers, including colorectal cancer, gastric cancer, liver cancer and pancreatic cancer. Table 8 matches each of the pathways with a figure from FIGS. 1-23.

TABLE 8
Summary of pathways of glycobiomarkers of GI cancers

| | | | CRC | | | | | Pancreatic | |
|---|---|---|---|---|---|---|---|---|---|
| | | | (Colo- | | | | | Adeno- | |
| | | | rectal | | Gastric | | Liver | carcinoma | |
| | | | cancer) | | cancer | | cancer | (PADC) | |
| # | Pathway Name | FIG# | F | M | F | M | M | F | M |
| 1 | Adherens Junction | 1 | | | XX | | | | |
| | Assembly (Nectin) | | | | | | | | |
| 2 | Bradykinin Effects | 2 | | | | | | XX | |
| | in Inflammation | | | | | | | | |
| 3 | Coagulation | 3 | XX | XX | XX | XX | XX | XX | XX |
| | Cascade | | | | | | | | |
| 4 | Complement | 4 | | | | XX | XX | XX | |
| | Activation by | | | | | | | | |
| | Lectin | | | | | | | | |
| 5 | Complement | 5 | XX | | XX | XX | XX | XX | XX |
| | Activation in | | | | | | | | |
| | Macular | | | | | | | | |
| | Degeneration | | | | | | | | |
| 6 | Complement | 6 | | | | XX | XX | XX | |
| | Alternative | | | | | | | | |
| | Pathway | | | | | | | | |
| 7 | Complement | 7 | XX | | | XX | XX | XX | XX |
| | Cascade | | | | | | | | |
| | Activation by | | | | | | | | |
| | Pentraxins | | | | | | | | |
| 8 | Complement | 8 | XX | | | XX | XX | XX | |
| | Classical Pathway | | | | | | | | |
| 9 | Focal Junction | 9 | XX | XX | XX | XX | | | |
| | Assembly | | | | | | | | |
| 10 | Glycolysis | 10 | XX | | XX | XX | | | |
| 11 | Histidine-Rich | 11 | | XX | | | XX | XX | |
| | Glycoprotein | | | | | | | | |
| | (HRG) | | | | | | | | |
| 12 | Lipogenesis | 12 | | | | | | | XX |
| | Regulation in | | | | | | | | |
| | Adipocytes | | | | | | | | |
| 13 | Microtubule | 13 | | | XX | | | | |
| | Cytoskeleton | | | | | | | | |
| 14 | Neutrophil | 14 | | | XX | | | | |
| | Activation via | | | | | | | | |
| | Adherence on | | | | | | | | |
| | Endothelial Cells | | | | | | | | |
| 15 | Plasmin Effects in | 15 | | XX | | XX | XX | XX | |
| | Inflammation | | | | | | | | |
| 16 | Platelet Activation | 16 | XX | XX | XX | | | | |
| | via Adhesion | | | | | | | | |
| | Molecules | | | | | | | | |
| 17 | Platelet Activation | 17 | | XX | | | | | |
| | via GPCR | | | | | | | | |
| | Signaling | | | | | | | | |
| 18 | Positive Acute | 18 | | | | | | XX | XX |
| | Phase Proteins | | | | | | | | |
| | Synthesis | | | | | | | | |
| 19 | Protein Folding | 19 | | XX | XX | | | | |
| 20 | Scavenger | 20 | XX | XX | XX | XX | | | XX |
| | Receptors in | | | | | | | | |
| | Platelet Activation | | | | | | | | |
| 21 | Scavenger | 21 | XX | XX | | | XX | | XX |
| | Receptors in | | | | | | | | |
| | Platelet | | | | | | | | |
| | Aggregation | | | | | | | | |
| 22 | TAM Receptors in | 22 | XX | XX | | | XX | | XX |
| | Platelet | | | | | | | | |
| | Aggregation | | | | | | | | |
| 23 | Vascular | 23 | | | | | | | XX |
| | Endothelial Cell | | | | | | | | |
| | Activation by | | | | | | | | |
| | Blood Coagulation | | | | | | | | |
| | Factors | | | | | | | | |

M—Male;
F—Female
XX—denotes that the pathway is affected.

Each of the pathways listed in Table 8 (as defined in more detail in FIGS. 1-23 by proteins which play a role in the pathway) can be used as a biomarker in a number of various tests. Various proteins from each of the pathway, including those listed in tables 1- 7 and other proteins as shown in FIGS. 1-23, can be included as representative biomarkers in screening, monitoring and treating GI cancer patients.

Further embodiments provide diagnostic methods based on any of the biomarkers in Tables 1, 2, 3, 4, 5, 6, 7 and/or 8, as may be further modified based on FIGS. 1-23. Various methods are contemplated and may include an immunoassay, biochip assay, nanoassay in which at least one panel with at least one or more biomarkers from Tables 1-8 is used, as may be further modified with any of additional protein biomarkers shown in FIGS. 1-23.

At least in some of these methods, a sample is obtained from a patient. This sample may be a human tissue biopsy or biosample including pancreas biopsy sample, gastrointestinal sample, blood sample, plasma sample, serum sample, circulating tumor cells sample, tear sample, saliva sample, sperm sample, urine sample, fecal sample and hair sample or any other human biospecimen. The sample is then screened to obtain a protein profile and to determine whether the protein profile in the sample matches at least partially a profile from any of Tables 1, 2, 3, 4, 5, 6, or 7. The abnormal proteins in the patient's profile are also analyzed to determine which of the pathways are affected, including the pathways shown in Tables 1-8.

Suitable screening methods may include chromatography, gas chromatography, liquid chromatography, mass spectrometry, ELISA, antibody linkage, immunoassay, biochip assay, microarray, nanoassay, spectroscopy, a multiplex molecular assay or techniques which utilize a fluorescent, enzyme, radioactive, metallic, biotin, chemiluminescent, bioluminescent molecule assay. Suitable methods further include a combination of a detection techniques of nucleic acids and proteins or peptides.

In some methods, at least one biomarker and/or glycobiomarker of Tables 1-8 is immobilized on a solid support. In some methods, the testing is conducted by reacting the patient's sample with at least one anybody or protein chemistry based reagent specific to at least one biomarker and/or glycobiomarker of Tables 1-8.

In further embodiments, the testing is conducted by reacting the patient's sample with at least one protein specific to at least one biomarker and/or glycobiomarker of Tables 1-8. In some embodiments, the testing is conducted by reacting the patient's sample with a synthetic compound or probe which reacts with at least one protein specific to at least one biomarker and/or glycobiomarker of Tables 1-8.

Further embodiments include a method for diagnosing, monitoring and treating a GI cancer, the method comprising obtaining a blood or plasma sample from a patient in need of the treatment, analyzing glycoproteins in the sample, creating a profile of pathways and glycoproteins for the patient, and comparing the profile to the profiles of glycobiomarkers and pathways of Tables 1-8.

In some embodiments, a screening can be conducted with a patient's sample without protein extraction. In further embodiments, proteins are isolated from the patient's sample, such as a blood or plasma sample, and a test is conducted with the isolated proteins. In some embodiments, all proteins in the sample are analyzed. In other embodiments, the analysis is conducted only for proteins which are glycosylated. In further embodiments, only GlcNac glycosylated proteins are analyzed.

The advantages of these screening methods include: these tests are non-invasive and they can be conducted in a very short period of time. In some embodiments, the same test can be repeated several times within a period of time to monitor progression of a GI cancer and/or evaluate the efficiency of a treatment plan.

Further embodiments provide Multiplex Molecular Diagnostic Protein assays, a combination of protein assay and assay based on detection of DNA or RNA, and kits, including an immunoassay, biochip assay, nanoassay and molecular assay. In some embodiments, an assay detects protein biomarker and/or glycobiomarkers or peptides derived from the biomarker and/or glycobiomarkers from any of Tables 1-8 and FIGS. 1-24.

In further embodiments, a patient's sample is reacted with a set of antibodies, each of which is selectively specific for at least one biomarker and/or glycobiomarker from Tables 1, 2, 3, 4, 5, 6, 7 or 8. The complex between an antibody and a glycobiomarker or a biomarker is then may be detected with a second antibody conjugated to a detection molecule.

Further embodiments include methods for detecting and monitoring a GI cancer. In these methods, a patient's sample is tested for expression of at least some biomarker and/or glycobiomarkers listed in Table 2, 3, 4, 5, 6, 7 or 8.

Further embodiments include methods in which patient's response to therapy, such as for example surgery, radiation, immunotherapy or chemotherapy, is monitored with testing a patient's sample for expression of at least some biomarker and/or glycobiomarkers listed in Tables 1, 2, 3, 4, 5, 6, 7 and 8. Other applications include detecting a recurrent or residual GI cancer by testing a patient's sample for expression of at least some biomarker and/or glycobiomarkers listed in Tables 1, 2, 3, 4, 5, 6 and/or 7.

Other applications include screening of genetically predisposed individuals for a GI caner by testing the individual's sample for expression of at least some biomarkers and/or glycobiomarkers listed in Tables 1, 2, 3, 4, 5, 6 and/or 7. Such genetically predisposed individuals include, but not limited, to BRCA mutation carriers; PALB2 mutation carriers; p16 mutation carriers; Lynch syndrome patients; Peutz-Jeghers syndrome patients; and individuals with a family history of a GI cancer.

In some methods, a biochip comprising a set of at least one or more biomarker and/or glycobiomarkers listed in Tables 1, 2, 3, 4, 5, 6 and/or 7 can be used as a robust and sensitive tool to monitor a GI cancer progression and response to therapy. These biochips can be also used as a biomarker or molecular modality for drug development or drug optimization.

In some embodiments, a patient can be screened and evaluated based on a test conducted with the patient's sample and a panel of biomarker and/or glycobiomarkers and pathways which include at least one or more biomarkers listed in Tables 1, 2, 3, 4, 5, 6, 7 and/or 8.

This invention also provides compositions and methods for selective detection of pancreatic diseases and/or disorders of the pancreas, including pancreatic cancer, pancreatitis, acute pancreatitis, chronic pancreatitis, hereditary pancreatitis, autoimmune pancreatitis, and pancreatic neoplasm. It also provides compositions and methods for monitoring progression of a pancreatic disease and/or disorder of the pancreas, including, but not limited to, pancreatic cancer, pancreatitis, and autoimmune pancreatitis

The invention provides a panel of pancreatic disease biomarkers. These biomarkers may include glycosylated biomarkers. In some embodiments, a panel of biomarkers include at least one or more glycosylated biomarkers listed in Tables 5A, 5, 6A, and 6. In other embodiments, a panel of biomarkers include all biomarkers listed in Tables 5A, 5, 6A, and 6. Further embodiments include a panel which comprises at least one or more biomarkers as listed in Table 9. In further embodiments, a panel includes a combination of at least one or more biomarkers from Table 9 and at least one or more biomarkers from any of the tables 5A, 5, 6A, and 6.

In some embodiments, a panel of biomarkers includes at least one or more proteins listed in Table 9. In other embodiments, a panel of biomarkers includes all proteins listed in Table 9. Further embodiments include a panel which comprises at least one or more glycosylated biomarkers as listed in any of the Tables 5A, 5, 6A, and 6.. In further embodiments, a panel includes a combination of at least one or more biomarkers from Tables 5A, 5, 6A, 6 and 9.

TABLE 9
Proteins differentially expressed in human plasma of pancreatic adenocarcinoma patients

| | |
|---|---|
| 14-3-3 protein gamma OS = Homo sapiens GN = YWHAG PE = 1 | sp\|P61981\|1433G_HUMAN |
| SV = 2 | |
| 14-3-3 protein zeta/delta OS = Homo sapiens GN = YWHAZ PE = 1 | sp\|P63104\|1433Z_HUMAN |
| SV = 1 | |
| 78 kDa glucose-regulated protein OS = Homo sapiens | sp\|P11021\|GRP78_HUMAN |
| GN = HSPA5 PE = 1 SV = 2 | |
| Actin, cytoplasmic 2 OS = Homo sapiens GN = ACTG1 PE = 1 SV = 1 | sp\|P63261\|ACTG_HUMAN |
| Alpha-1-acid glycoprotein 2 OS = Homo sapiens GN = ORM2 | sp\|P19652\|A1AG2_HUMAN |
| PE = 1 SV = 2 | |
| Alpha-2-macroglobulin OS = Homo sapiens GN = A2M PE = 1 | sp\|P01023\|A2MG_HUMAN |
| SV = 3 | |
| Alpha-actinin-1 OS = Homo sapiens GN = ACTN1 PE = 1 SV = 2 | sp\|P12814\|ACTN1_HUMAN |
| Alpha-enolase OS = Homo sapiens GN = ENO1 PE = 1 SV = 2 | sp\|P06733\|ENOA_HUMAN |
| Alpha-N-acetylglucosaminidase OS = Homo sapiens | sp\|P54802\|ANAG_HUMAN |
| GN = NAGLU PE = 1 SV = 2 | |
| Aminopeptidase N OS = Homo sapiens GN = ANPEP PE = 1 SV = 4 | sp\|P15144\|AMPN_HUMAN |
| Angiogenin OS = Homo sapiens GN = ANG PE = 1 SV = 1 | sp\|P03950\|ANGI_HUMAN |
| Apolipoprotein A-I OS = Homo sapiens GN = APOA1 PE = 1 SV = 1 | sp\|P02647\|APOA1_HUMAN |
| Apolipoprotein A-II OS = Homo sapiens GN = APOA2 PE = 1 SV = 1 | sp\|P02652\|APOA2_HUMAN |
| Apolipoprotein C-III OS = Homo sapiens GN = APOC3 PE = 1 SV = 1 | sp\|P02656\|APOC3_HUMAN |
| Apolipoprotein C-IV OS = Homo sapiens GN = APOC4 PE = 1 SV = 1 | sp\|P55056\|APOC4_HUMAN |
| Apolipoprotein F OS = Homo sapiens GN = APOF PE = 1 SV = 2 | sp\|Q13790\|APOF_HUMAN |
| Beta-2-microglobulin OS = Homo sapiens GN = B2M PE = 1 SV = 1 | sp\|P61769\|B2MG_HUMAN |
| Beta-enolase OS = Homo sapiens GN = ENO3 PE = 1 SV = 5 | sp\|P13929\|ENOB_HUMAN |
| Cadherin-5 OS = Homo sapiens GN = CDH5 PE = 1 SV = 5 | sp\|P33151\|CADH5_HUMAN |
| Calmodulin OS = Homo sapiens GN = CALM1 PE = 1 SV = 2 | sp\|P62158\|CALM_HUMAN |
| Calreticulin OS = Homo sapiens GN = CALR PE = 1 SV = 1 | sp\|P27797\|CALR_HUMAN |
| Carbonic anhydrase 1 OS = Homo sapiens GN = CA1 PE = 1 SV = 2 | sp\|P00915\|CAH1_HUMAN |
| Carbonic anhydrase 2 OS = Homo sapiens GN = CA2 PE = 1 SV = 2 | sp\|P00918\|CAH2_HUMAN |
| Cartilage oligomeric matrix protein OS = Homo sapiens | sp\|P49747\|COMP_HUMAN |
| GN = COMP PE = 1 SV = 2 | |
| Catalase OS = Homo sapiens GN = CAT PE = 1 SV = 3 | sp\|P04040\|CATA_HUMAN |
| Cathepsin D OS = Homo sapiens GN = CTSD PE = 1 SV = 1 | sp\|P07339\|CATD_HUMAN |
| CD5 antigen-like OS = Homo sapiens GN = CD5L PE = 1 SV = 1 | sp\|O43866\|CD5L_HUMAN |
| Cell surface glycoprotein MUC18 OS = Homo sapiens | sp\|P43121\|MUC18_HUMAN |
| GN = MCAM PE = 1 SV = 2 | |
| Chloride intracellular channel protein 1 OS = Homo sapiens | sp\|O00299\|CLIC1_HUMAN |
| GN = CLIC1 PE = 1 SV = 4 | |
| Clusterin OS = Homo sapiens GN = CLU PE = 1 SV = 1 | sp\|P10909\|CLUS_HUMAN |
| Coagulation factor X OS = Homo sapiens GN = F10 PE = 1 SV = 2 | sp\|P00742\|FA10_HUMAN |
| Coagulation factor XII OS = Homo sapiens GN = F12 PE = 1 SV = 3 | sp\|P00748\|FA12_HUMAN |
| Cofilin-1 OS = Homo sapiens GN = CFL1 PE = 1 SV = 3 | sp\|P23528\|COF1_HUMAN |
| Collectin-10 OS = Homo sapiens GN = COLEC10 PE = 2 SV = 2 | sp\|Q9Y6Z7\|COL10_HUMAN |
| Complement C4-B OS = Homo sapiens GN = C4B PE = 1 SV = 1 | sp\|P0C0L5\|CO4B_HUMAN |
| Coronin-1A OS = Homo sapiens GN = CORO1A PE = 1 SV = 4 | sp\|P31146\|COR1A_HUMAN |
| Corticosteroid-binding globulin OS = Homo sapiens | sp\|P08185\|CBG_HUMAN |
| GN = SERPINA6 PE = 1 SV = 1 | |
| C-reactive protein OS = Homo sapiens GN = CRP PE = 1 SV = 1 | sp\|P02741\|CRP_HUMAN |
| Creatine kinase M-type OS = Homo sapiens GN = CKM PE = 1 | sp\|P06732\|KCRM_HUMAN |
| SV = 2 | |
| Cystatin-C OS = Homo sapiens GN = CST3 PE = 1 SV = 1 | sp\|P01034\|CYTC_HUMAN |
| Cysteine-rich secretory protein 3 OS = Homo sapiens | sp\|P54108\|CRIS3_HUMAN |
| GN = CRISP3 PE = 1 SV = 1 | |
| EGF-containing fibulin-like extracellular matrix protein 1 | sp\|Q12805\|FBLN3_HUMAN |
| OS = Homo sapiens GN = EFEMP1 PE = 1 SV = 2 | |
| Fermitin family homolog 3 OS = Homo sapiens GN = FERMT3 | sp\|Q86UX7\|URP2_HUMAN |
| PE = 1 SV = 1 | |
| Fibrinogen alpha chain OS = Homo sapiens GN = FGA PE = 1 SV = 2 | sp\|P02671\|FIBA_HUMAN |
| Fibrinogen beta chain OS = Homo sapiens GN = FGB PE = 1 SV = 2 | sp\|P02675\|FIBB_HUMAN |
| Fibrinogen gamma chain OS = Homo sapiens GN = FGG PE = 1 | sp\|P02679\|FIBG_HUMAN |
| SV = 3 | |
| Filamin-A OS = Homo sapiens GN = FLNA PE = 1 SV = 4 | sp\|P21333\|FLNA_HUMAN |
| Flavin reductase (NADPH) OS = Homo sapiens GN = BLVRB PE = 1 | sp\|P30043\|BLVRB_HUMAN |
| SV = 3 | |
| Fructose-bisphosphate aldolase A OS = Homo sapiens | sp\|P04075\|ALDOA_HUMAN |
| GN = ALDOA PE = 1 SV = 2 | |
| Galectin-3-binding protein OS = Homo sapiens GN = LGALS3BP | |
| PE = 1 SV = 1 | sp\|Q08380\|LG3BP_HUMAN |
| Gamma-glutamyl hydrolase OS = Homo sapiens GN = GGH PE = 1 | sp\|Q92820\|GGH_HUMAN |
| SV = 2 | |
| Glutathione S-transferase omega-1 OS = Homo sapiens | sp\|P78417\|GSTO1_HUMAN |
| GN = GSTO1 PE = 1 SV = 2 | |
| Glyceraldehyde-3-phosphate dehydrogenase OS = Homo | sp\|P04406\|G3P_HUMAN |
| sapiens GN = GAPDH PE = 1 SV = 3 | |
| Haptoglobin OS = Homo sapiens GN = HP PE = 1 SV = 1 | sp\|P00738\|HPT_HUMAN |
| Hemoglobin subunit alpha OS = Homo sapiens GN = HBA1 PE = 1 | sp\|P69905\|HBA_HUMAN |
| SV = 2 | |
| Hemoglobin subunit beta OS = Homo sapiens GN = HBB PE = 1 | sp\|P68871\|HBB_HUMAN |
| SV = 2 | |
| Hepatocyte growth factor-like protein OS = Homo sapiens | sp\|P26927\|HGFL_HUMAN |
| GN = MST1 PE = 1 SV = 2 | |
| Ig gamma-3 chain C region OS = Homo sapiens GN = IGHG3 | sp\|P01860\|IGHG3_HUMAN |
| PE = 1 SV = 2 | |
| Ig kappa chain C region OS = Homo sapiens GN = IGKC PE = 1 | sp\|P01834\|IGKC_HUMAN |
| SV = 1 | |
| Ig mu chain C region OS = Homo sapiens GN = IGHM PE = 1 SV = 3 | sp\|P01871\|IGHM_HUMAN |
| Immunoglobulin lambda-like polypeptide 5 OS = Homo sapiens | sp\|B9A064\|IGLL5_HUMAN |
| GN = IGLL5 PE = 2 SV = 2 | (+4) |
| Insulin-like growth factor II OS = Homo sapiens GN = IGF2 PE = 1 | sp\|P01344\|IGF2_HUMAN |
| SV = 1 | |
| Insulin-like growth factor-binding protein 3 OS = Homo sapiens | sp\|P17936\|IBP3_HUMAN |
| GN = IGFBP3 PE = 1 SV = 2 | |
| Intercellular adhesion molecule 1 OS = Homo sapiens | sp\|P05362\|ICAM1_HUMAN |
| GN = ICAM1 PE = 1 SV = 2 | |
| Keratin, type II cytoskeletal 1 OS = Homo sapiens GN = KRT1 | sp\|P04264\|K2C1_HUMAN |
| PE = 1 SV = 6 | |
| Keratin, type II cytoskeletal 2 epidermal OS = Homo sapiens | sp\|P35908\|K22E_HUMAN |
| GN = KRT2 PE = 1 SV = 2 | |
| L-lactate dehydrogenase A chain OS = Homo sapiens GN = LDHA | sp\|P00338\|LDHA_HUMAN |
| PE = 1 SV = 2 | |
| L-lactate dehydrogenase B chain OS = Homo sapiens GN = LDHB | sp\|P07195\|LDHB_HUMAN |
| PE = 1 SV = 2 | |
| Lysosome-associated membrane glycoprotein 1 OS = Homo | sp\|P11279\|LAMP1_HUMAN |
| sapiens GN = LAMP1 PE = 1 SV = 3 | |
| Lysozyme C OS = Homo sapiens GN = LYZ PE = 1 SV = 1 | sp\|P61626\|LYSC_HUMAN |
| Mannan-binding lectin serine protease 2 OS = Homo sapiens | sp\|O00187\|MASP2_HUMAN |
| GN = MASP2 PE = 1 SV = 4 | |
| Mannosyl-oligosaccharide 1,2-alpha-mannosidase IA | sp\|P33908\|MA1A1_HUMAN |
| OS = Homo sapiens GN = MAN1A1 PE = 1 SV = 3 | |
| Multiple epidermal growth factor-like domains protein 8 | sp\|Q7Z7M0\|MEGF8_HUMAN |
| OS = Homo sapiens GN = MEGF8 PE = 1 SV = 2 | |
| Neural cell adhesion molecule 1 OS = Homo sapiens | sp\|P13591\|NCAM1_HUMAN |
| GN = NCAM1 PE = 1 SV = 3 | |
| Neural cell adhesion molecule L1-like protein OS = Homo | sp\|O00533\|CHL1_HUMAN |
| sapiens GN = CHL1 PE = 1 SV = 4 | |
| Neutrophil defensin 1 OS = Homo sapiens GN = DEFA1 PE = 1 | sp\|P59665\|DEF1_HUMAN |
| SV = 1 | (+1) |
| Peroxiredoxin-2 OS = Homo sapiens GN = PRDX2 PE = 1 SV = 5 | sp\|P32119\|PRDX2_HUMAN |
| Phosphatidylinositol-glycan-specific phospholipase D | sp\|P80108\|PHLD_HUMAN |
| OS = Homo sapiens GN = GPLD1 PE = 1 SV = 3 | |
| Pigment epithelium-derived factor OS = Homo sapiens | sp\|P36955\|PEDF_HUMAN |
| GN = SERPINF1 PE = 1 SV = 4 | |
| Plasma alpha-L-fucosidase OS = Homo sapiens GN = FUCA2 | sp\|Q9BTY2\|FUCO2_HUMAN |
| PE = 1 SV = 2 | |
| Plasma kallikrein OS = Homo sapiens GN = KLKB1 PE = 1 SV = 1 | sp\|P03952\|KLKB1_HUMAN |
| Platelet glycoprotein Ib alpha chain OS = Homo sapiens | sp\|P07359\|GP1BA_HUMAN |
| GN = GP1BA PE = 1 SV = 1 | |
| Pleckstrin OS = Homo sapiens GN = PLEK PE = 1 SV = 3 | sp\|P08567\|PLEK_HUMAN |
| Pregnancy zone protein OS = Homo sapiens GN = PZP PE = 1 | sp\|P20742\|PZP_HUMAN |
| SV = 4 | |
| Procollagen C-endopeptidase enhancer 1 OS = Homo sapiens | sp\|Q15113\|PCOC1_HUMAN |
| GN = PCOLCE PE = 1 SV = 2 | |
| Protein S100-A8 OS = Homo sapiens GN = S100A8 PE = 1 SV = 1 | sp\|P05109\|S10A8_HUMAN |
| Pyruvate kinase isozymes M1/M2 OS = Homo sapiens GN = PKM | sp\|P14618\|KPYM_HUMAN |
| PE = 1 SV = 4 | |
| Ras suppressor protein 1 OS = Homo sapiens GN = RSU1 PE = 1 | sp\|Q15404\|RSU1_HUMAN |
| SV = 3 | |
| Receptor-type tyrosine-protein phosphatase eta OS = Homo | sp\|Q12913\|PTPRJ_HUMAN |
| sapiens GN = PTPRJ PE = 1 SV = 3 | |
| Retinol-binding protein 4 OS = Homo sapiens GN = RBP4 PE = 1 | sp\|P02753\|RET4_HUMAN |
| SV = 3 | |
| Scavenger receptor cysteine-rich type 1 protein M130 | sp\|Q86VB7\|C163A_HUMAN |
| OS = Homo sapiens GN = CD163 PE = 1 SV = 2 | |
| Secreted phosphoprotein 24 OS = Homo sapiens GN = SPP2 | sp\|Q13103\|SPP24_HUMAN |
| PE = 1 SV = 1 | |
| Serotransferrin OS = Homo sapiens GN = TF PE = 1 SV = 3 | sp\|P02787\|TRFE_HUMAN |
| Sex hormone-binding globulin OS = Homo sapiens GN = SHBG | sp\|P04278\|SHBG_HUMAN |
| PE = 1 SV = 2 | |
| Talin-1 OS = Homo sapiens GN = TLN1 PE = 1 SV = 3 | sp\|Q9Y490\|TLN1_HUMAN |
| Transgelin-2 OS = Homo sapiens GN = TAGLN2 PE = 1 SV = 3 | sp\|P37802\|TAGL2_HUMAN |
| Triosephosphate isomerase OS = Homo sapiens GN = TPI1 PE = 1 | sp\|P60174\|TPIS_HUMAN |
| SV = 3 | |
| Tropomyosin alpha-4 chain OS = Homo sapiens GN = TPM4 | sp\|P67936\|TPM4_HUMAN |
| PE = 1 SV = 3 | |
| Vasodilator-stimulated phosphoprotein OS = Homo sapiens | sp\|P50552\|VASP_HUMAN |
| GN = VASP PE = 1 SV = 3 | |
| Vinculin OS = Homo sapiens GN = VCL PE = 1 SV = 4 | sp\|P18206\|VINC_HUMAN |
| Vitamin K-dependent protein S OS = Homo sapiens GN = PROS1 | sp\|P07225\|PROS_HUMAN |
| PE = 1 SV = 1 | |

Other embodiments provide kits which comprise at least one panel as described above in connection with Tables 5A, 5, 6A, 6 and 9. Such kits may further comprise a biochip.

Various methods are contemplated and may include an immunoassay, biochip assay, nanoassay in which at least one panel with at least one or more biomarkers from

Tables 5A, 5, 6A and 6 or at least one or more biomarkers from Table 9 are used. At least in some of these methods, a sample is obtained from a patient. This sample may be a human tissue biopsy or biosample including pancreas biopsy sample, gastrointestinal sample, blood sample, plasma sample, serum sample, circulating tumor cells sample, tear sample, saliva sample, sperm sample, urine sample, fecal sample and hair sample or any other human biospecimen.

The sample is then screened for presence of at least one or more glycosylated markers listed in Tables 5A, 5, 6A, 6 and/or for presence of at least one or more protein markers listed in Table 9.

Suitable screening methods may include chromatography, gas chromatography, liquid chromatography, mass spectrometry, ELISA, antibody linkage, immunoassay, biochip assay, microarray, nanoassay, spectroscopy, a multiplex molecular assay or techniques which utilize a fluorescent, enzyme, radioactive, metallic, biotin, chemiluminescent, bioluminescent molecule assay. Suitable methods further include a combination of a detection techniques of nucleic acids and proteins or peptides. In some methods, at least one biomarker and/or glycobiomarker of Tables 5A, 5, 6A, 6 and 9 is immobilized on a solid support. In some methods, the testing is conducted by reacting the patient's sample with at least one anybody or protein chemistry based reagent specific to at least one biomarker and/or glycobiomarker of Tables 5A, 5, 6A, 6 and 9. In further embodiments, the testing is conducted by reacting the patient's sample with at least one protein specific to at least one biomarker and/or glycobiomarker of Tables 5A, 5, 6A, 6 and 9.

In some embodiments, the testing is conducted by reacting the patient's sample with a synthetic compound or probe which react with at least one protein specific to at least one biomarker and/or glycobiomarker of Tables 5A, 5, 6A, 6 and 9.

Further embodiments include a method for treating a disorder of the pancreas, the method comprising obtaining a sample from a mammal in need of the treatment and testing the sample for at least one or more biomarker and/or glycobiomarker selected from Tables 5A, 5, 6A, 6 and 9.

A method is provided for determining a state or probability of a pancreatic disorder, the method comprising: (a) determining the level of one or more biomarker and/or glycobiomarker selected from Tables 5A, 5, 6A, 6 and 9, and (b) the level of CA 19-9.

A method is provided for determining a state or probability of a pancreatic disorder, the method comprising: (a) determining the level of one or more biomarker and/or glycobiomarker selected from Tables 5A, 5, 6A, 6 and 9 and (b) the level of amylase.

A method is provided for determining a state or probability of a pancreatic disorder, the method comprising: (a) determining the level of one or more biomarker and/or glycobiomarker selected from Tables 5A, 5, 6A, 6 and 9, and (b) the level of glycosylated protein.

A method is provided for determining a state or probability of a pancreatic disorder, the method comprising: (a) determining the level of one or more biomarker and/or glycobiomarker selected from Tables 5A, 5, 6A, 6 and 9, and (b) the level of RNA or DNA.

A method is provided for determining a state or probability of a pancreatic disorder, the method comprising: (a) determining the level of one or more biomarker and/or glycobiomarker selected from Tables 5A, 5, 6A, 6 and 9, and (b) the level of virus or viral infection of the patient.

In some embodiments, a screening can be conducted with a patient's sample without protein extraction. In further embodiments, proteins are extracted from the patient's sample and a test is conducted with the extracted proteins. In some embodiments, all proteins in the sample are analyzed. In other embodiments, the analysis is conducted only for proteins which are abnormally glycosylated.

The advantages of these screening methods include: these tests are non-invasive and they can be conducted in a very short period of time. In some embodiments, the same test can be repeated several times within a period of time to monitor progression of a pancreatic disease and/or access the efficiency of a treatment plan.

Further embodiments provide Multiplex Molecular Diagnostic Protein assays, a combination of protein assay and assay based on detection of DNA or RNA, and kits, including an immunoassay, biochip assay, nanoassay and molecular assay. In some embodiments an assay detects protein biomarker and/or glycobiomarkers or peptides derived from the biomarker and/or glycobiomarkers from Tables 5A, 5, 6A, and 6. In other embodiments, an assay detects biomarkers or peptides derived from biomarkers from Table 9. In other embodiments, an assay detects biomarkers or peptides derived from biomarkers from Table 9. In other embodiments, an assay detects biomarkers or peptides derived from biomarkers from Table 9.

In further embodiments, a patient's sample is reacted with a set of antibodies, each of which is selectively specific for at least one biomarker and/or glycobiomarker from Tables 5A, 5, 6A, 6 and 9. The complex between an antibody and a glycobiomarker or a biomarker is then may be detected with a second antibody conjugated to a detection molecule.

Further embodiments include methods for detecting and monitoring a pancreatic disease, including pancreatic cancer, pancreatitis, and autoimmune pancreatitis. In these methods, a patient's sample is tested for expression of at least some biomarker and/or glycobiomarkers listed in Tables 5A, 5, 6A, 6 and 9. Further embodiments include methods in which patient's response to therapy is monitored with testing a patient's sample for expression of at least some biomarker and/or glycobiomarkers listed in Tables 5A, 5, 6A, 6 and 9. Other applications include detecting a recurrent or residual pancreatic disease by testing a patient's sample for expression of at least some biomarker and/or glycobiomarkers listed in Tables 5A, 5, 6A, 6 and 9.

Other applications include screening of genetically predisposed individuals for a pancreatic disease by testing the individual's sample for expression of at least some biomarkers and/or glycobiomarkers listed in Tables 5A, 5, 6A, 6 and 9. Such genetically predisposed individuals include, but not limited, to BRCA mutation carriers; PALB2 mutation carriers; p16 mutation carriers; Lynch syndrome patients; Peutz-Jeghers syndrome patients; and individuals with a family history of pancreatic cancer cases.

In some methods, a biochip comprising a set of at least one or more biomarker and/or glycobiomarkers listed in Tables 5A, 5, 6A, 6 and 9 can be used as a robust and sensitive tool to monitor disease progression and response to therapy. These biochips can be also used as a biomarker or molecular modality for drug development or drug optimization.

Other applications include tests conducted for detection and measurement of biomarker and/or glycobiomarkers which include at least one or more biomarkers listed in Tables 5A, 5, 6A, 6 and 9. Such tests may include verification and support of clinical and operative decision-making process and management of pancreatic cyst neoplasms.

Further embodiments provide methods for treating a disorder of the pancreas, the method comprising obtaining a sample from a mammal in need of the treatment and testing the sample for at least one or more biomarker and/or glycobiomarker selected from Tables 5A, 5, 6A, 6 and 9.

The invention will be now further explained by the following non-limiting examples.

### EXAMPLE 1

Human plasma was obtained from cancer patients. Human plasma samples from healthy control individuals were used as controls. All patients and control individuals have provided informed consent, and collection of human samples was approved by the local Review Board. Glycosylated forms of proteins were isolated from human plasma as it has been described before (Khidekel N, Ficarro S B, Peters E C, Hsieh-Wilson L C. “Exploring the O-GlcNAc proteome: direct identification of O-GlcNAc-modified proteins from the brain”. PNAS 2004 Sep. 7; 101(36):13132-7. Yi W, Clark P M, Mason D E, Keenan M C, Hill C, Goddard W A 3rd, Peters E C, Driggers E M, Hsieh-Wilson L C. “Phosphofructokinase 1 glycosylation regulates cell growth and metabolism”. Science. 2012 Aug. 24; 337(6097):975-80.) Then isolated glycosylated forms of proteins from human plasma were used for the proteomics analysis.

Mass Spectrometry of protein expression profiling was performed according to the established protocol. Briefly, each isolated sample was processed by SDS-PAGE, using a 10% Bis-Tris NuPAGE gel (Invitrogen) with the MES buffer system. The entire mobility region was excised and processed by in-gel digestion using a robot (ProGest, DigiLab) with the following protocol: washed with 25 mM ammonium bicarbonate followed by acetonitrile; reduced with 10 mM dithiothreitol at 60° C. followed by alkylation with 50 mM iodoacetamide at RT. digested with trypsin (Promega) at 37° C. for 4 h, quenched with formic acid and the supernatant was analyzed directly without further processing. Mass Spectrometry of each digested sample was analyzed by nano LC-MS/MS with a ThermoFisher EASY-nLC 1000 HPLC system interfaced to a ThermoFisher Q Exactive mass spectrometer.

A sample was then loaded on a trapping column and eluted over a 75 μm×150 mm analytical column (Thermo Fisher P/N 164568) at 300 nL/min using a 4 hr reverse phase gradient; both columns were packed with Acclaim PepMap 100 Å, 3 3 μm resin (Thermo Scientific). The mass spectrometer was operated in the data-dependent mode, with MS and MS/MS performed in the Orbitrap at 70,000 and 17,500 FWHM resolution respectively. The fifteen most abundant ions were selected for MS/MS. The data processing was analyzed using a Mascot. Mascot DAT files were parsed into the Scaffold software for validation, filtering and to create a nonredundant list per sample. The data was filtered using at 1% protein and peptide FDR and requiring at least two unique peptides per protein.

The list of proteins was then additionally analyzed with the ELSEVIER PATHWAY STUDIO software.

### EXAMPLE 2

Human plasma was obtained from cancer patients. Human plasma samples from healthy control individuals were used as controls. All patients and control individuals have provided informed consent, and collection of human samples was approved by the local Review Board. 10 μL of each plasma sample was processed using the Multiple Affinity Removal System (MARS specific for the 14 most abundant human plasma proteins (Agilent (P/N5188-6560)). The sample was processed using the vendor protocol. Depleted samples were buffer exchanged against HPLC grade water and quantified by Qubit fluorometry at a 1:10 dilution and % depletion was assessed. 20 μg of each sample was digested with trypsin using the following protocol: reduced with 10 mM dithiothreitol at 60° C. for 30 minutes in 25 mM Ammonium bicarbonate; alkylated with iodoacetamide for at 60° C. for 45 minutes in 25 mM Ammonium bicarbonate; digested overnight with sequencing grade trypsin at 37° C., enzyme: substrate ratio 1:20; quenched with formic acid. Digested samples were desalted using a Waters HLB μElution plate per the vendor protocol. Desalted samples were suspended in 100 μL of 0.1% TFA for analysis.

Mass Spectrometry of protein expression profiling was performed according to the established protocol. Briefly, each isolated sample was processed by SDS-PAGE, using a 10% Bis-Tris NuPAGE gel (Invitrogen) with the MES buffer system. The entire mobility region was excised and processed by in-gel digestion using a robot (ProGest, DigiLab) with the following protocol: washed with 25 mM ammonium bicarbonate followed by acetonitrile; reduced with 10 mM dithiothreitol at 60° C. followed by alkylation with 50 mM iodoacetamide at RT. digested with trypsin (Promega) at 37° C. for 4 h, quenched with formic acid and the supernatant was analyzed directly without further processing. Mass Spectrometry of each digested sample was analyzed by nano LC-MS/MS with a ThermoFisher EASY-nLC 1000 HPLC system interfaced to a ThermoFisher Q Exactive mass spectrometer.

A sample was then loaded on a trapping column and eluted over a 75 μm×150 mm analytical column (Thermo Fisher P/N 164568) at 300 nL/min using a 4 hr reverse phase gradient; both columns were packed with Acclaim PepMap 100 Å, 3 3 μm resin (Thermo Scientific). The mass spectrometer was operated in the data-dependent mode, with MS and MS/MS performed in the Orbitrap at 70,000 and 17,500 FWHM resolution respectively. The fifteen most abundant ions were selected for MS/MS. The data processing was analyzed using a Mascot. Mascot DAT files were parsed into the Scaffold software for validation, filtering and to create a nonredundant list per sample. The data was filtered using at 1% protein and peptide FDR and requiring at least two unique peptides per protein.

### A List of Abbreviations for FIGS. 1-23 and Tables 1, 2, 3, 4, 5, 6, 7 and 8

### Adherens Junction Assembly (Nectin)

| Name | Description |
|---|---|
| ACTN1 | actinin alpha 1 |
| CDC42 | cell division cycle 42 |
| CRK | v-crk avian sarcoma virus CT10 oncogene homolog |
| CTNNA1 | catenin alpha 1 |
| RAPGEF1 | Rap guanine nucleotide exchange factor 1 |
| LMO7 | LIM domain 7 |
| MLLT4 | myeloid/lymphoid or mixed-lineage leukemia; translocated |
| | to, 4 |
| MYO7B | myosin VIIB |
| RAC1 | ras-related C3 botulinum toxin substrate 1 (rho family, |
| | small GTP binding protein Rac1) |
| RAP1A | RAP1A, member of RAS oncogene family |
| SRC | SRC proto-oncogene, non-receptor tyrosine kinase |
| TJP1 | tight junction protein 1 |
| VASP | vasodilator-stimulated phosphoprotein |
| VCL | vinculin |
| VAV2 | vav guanine nucleotide exchange factor 2 |
| WAS | Wiskott-Aldrich syndrome |
| ZYX | zyxin |
| IQGAP1 | IQ motif containing GTPase activating protein 1 |
| WASF1 | WAS protein family member 1 |
| WASL | Wiskott-Aldrich syndrome like |
| KEAP1 | kelch like ECH associated protein 1 |
| WASF2 | WAS protein family member 2 |
| BAIAP2 | BAI1 associated protein 2 |
| SORBS1 | sorbin and SH3 domain containing 1 |
| EPN1 | epsin 1 |
| PARD3 | par-3 family cell polarity regulator |
| SSX2IP | synovial sarcoma, X breakpoint 2 interacting protein |

### Bradykinin Effects in Inflammation

| Name | Description |
|---|---|
| AGT | angiotensinogen |
| AGTR1 | angiotensin II receptor type 1 |
| AKT1 | v-akt murine thymoma viral oncogene homolog 1 |
| BDKRB2 | bradykinin receptor B2 |
| BDKRB1 | bradykinin receptor B1 |
| CPN1 | carboxypeptidase N subunit 1 |
| CPM | carboxypeptidase M |
| MAPK14 | mitogen-activated protein kinase 14 |
| ACE | angiotensin I converting enzyme |
| F11 | coagulation factor XI |
| F12 | coagulation factor XII |
| GNA11 | G protein subunit alpha 11 |
| GNAQ | G protein subunit alpha q |
| IL1B | interleukin 1 beta |
| IL1RAP | interleukin 1 receptor accessory protein |
| IL1A | interleukin 1 alpha |
| IL1R1 | interleukin 1 receptor type 1 |
| ITPR1 | inositol 1,4,5-trisphosphate receptor type 1 |
| KLK1 | kallikrein 1 |
| KLKB1 | kallikrein B1 |
| KNG1 | kininogen 1 |
| MAP3K1 | mitogen-activated protein kinase kinase kinase 1 |
| MMP1 | matrix metallopeptidase 1 |
| NFKBIA | NFKB inhibitor alpha |
| NOS3 | nitric oxide synthase 3 |
| NOS2 | nitric oxide synthase 2 |
| PDPK1 | 3-phosphoinositide dependent protein kinase 1 |
| PLAT | plasminogen activator, tissue type |
| PLAU | plasminogen activator, urokinase |
| PLAUR | plasminogen activator, urokinase receptor |
| PLG | plasminogen |
| PLCB3 | phospholipase C beta 3 |
| PRKCQ | protein kinase C theta |
| MAP2K3 | mitogen-activated protein kinase kinase 3 |
| MAP2K6 | mitogen-activated protein kinase kinase 6 |
| RAC1 | ras-related C3 botulinum toxin substrate 1 (rho family, |
| | small GTP binding protein Rac1) |
| XPNPEP2 | X-prolyl aminopeptidase (aminopeptidase P) 2, membrane- |
| | bound |
| PTGES | prostaglandin E synthase |
| PLCB1 | phospholipase C beta 1 |

### Coagulation Cascade

| Name | Description |
|---|---|
| SERPINC1 | serpin family C member 1 |
| F2 | coagulation factor II, thrombin |
| F5 | coagulation factor V |
| F9 | coagulation factor IX |
| F11 | coagulation factor XI |
| F10 | coagulation factor X |
| F3 | coagulation factor III, tissue factor |
| F12 | coagulation factor XII |
| F13A1 | coagulation factor XIII A chain |
| F8 | coagulation factor VIII |
| F7 | coagulation factor VII |
| KLKB1 | kallikrein B1 |
| KNG1 | kininogen 1 |
| PLAT | plasminogen activator, tissue type |
| PLG | plasminogen |
| PROC | protein C, inactivator of coagulation factors Va and |
| | VIIIa |
| PROS1 | protein S (alpha) |
| TFPI | tissue factor pathway inhibitor |
| THBD | thrombomodulin |
| PROZ | protein Z, vitamin K dependent plasma glycoprotein |
| FGL2 | fibrinogen like 2 |
| SERPINA10 | serpin family A member 10 |
| VKORC1 | vitamin K epoxide reductase complex subunit 1 |

### Complement Activation by Lectin

| Name | Description |
|---|---|
| C3 | complement component 3 |
| C4B | complement component 4B (Chido blood group) |
| C2 | complement component 2 |
| C3AR1 | complement component 3a receptor 1 |
| C5 | complement component 5 |
| C5AR1 | complement component 5a receptor 1 |
| C8A | complement component 8 alpha subunit |
| C6 | complement component 6 |
| C9 | complement component 9 |
| C7 | complement component 7 |
| MBL2 | mannose binding lectin 2 |
| MASP1 | mannan binding lectin serine peptidase 1 |
| MASP2 | mannan binding lectin serine peptidase 2 |
| C5AR2 | complement component 5a receptor 2 |

### Complement Activation in Macular Degeneration

| Name | Description |
|---|---|
| CFB | complement factor B |
| C3 | complement component 3 |
| C4B | complement component 4B (Chido blood group) |
| C2 | complement component 2 |
| C1R | complement C1r subcomponent |
| C1S | complement component 1, s subcomponent |
| C5 | complement component 5 |
| C5AR1 | complement component 5a receptor 1 |
| C8A | complement component 8 alpha subunit |
| C6 | complement component 6 |
| C9 | complement component 9 |
| C7 | complement component 7 |
| CLU | clusterin |
| CR1 | complement component 3b/4b receptor 1 (Knops blood group) |
| CRP | C-reactive protein, pentraxin-related |
| CD55 | CD55 molecule (Cromer blood group) |
| CFD | complement factor D (adipsin) |
| CFHR1 | complement factor H related 1 |
| CFH | complement factor H |
| CFI | complement factor I |
| MBL2 | mannose binding lectin 2 |
| CD46 | CD46 molecule |
| CFP | complement factor properdin |
| MASP1 | mannan binding lectin serine peptidase 1 |
| HTRA1 | HtrA serine peptidase 1 |
| TIMP3 | TIMP metallopeptidase inhibitor 3 |
| VTN | vitronectin |
| MASP2 | mannan binding lectin serine peptidase 2 |
| CFHR3 | complement factor H related 3 |

### Complement Alternative Pathway

| Name | Description |
|---|---|
| CFB | complement factor B |
| C3 | complement component 3 |
| C5 | complement component 5 |
| C5AR1 | complement component 5a receptor 1 |
| C8A | complement component 8 alpha subunit |
| C6 | complement component 6 |
| C9 | complement component 9 |
| C7 | complement component 7 |
| CR2 | complement component 3d receptor 2 |
| CFD | complement factor D (adipsin) |
| CFH | complement factor H |
| CFI | complement factor I |
| CFP | complement factor properdin |
| C5AR2 | complement component 5a receptor 2 |

### Complement Cascade Activation by Pentraxins

| Name | Description |
|---|---|
| APCS | amyloid P component, serum |
| APP | amyloid beta precursor protein |
| C1QA | complement component 1, q subcomponent, A chain |
| C3 | complement component 3 |
| C4B | complement component 4B (Chido blood group) |
| C2 | complement component 2 |
| C4BPA | complement component 4 binding protein alpha |
| C1S | complement component 1, s subcomponent |
| C5 | complement component 5 |
| C9 | complement component 9 |
| CRP | C-reactive protein, pentraxin-related |
| FCGR3A | Fc fragment of IgG receptor IIIa |
| CFH | complement factor H |
| MBL2 | mannose binding lectin 2 |
| MASP1 | mannan binding lectin serine peptidase 1 |
| PTX3 | pentraxin 3 |
| SNRNP70 | small nuclear ribonucleoprotein U1 subunit 70 |
| SNRPN | small nuclear ribonucleoprotein polypeptide N |

### Complement Classical Pathway

| Name | Description |
|---|---|
| C3 | complement component 3 |
| C4B | complement component 4B (Chido blood group) |
| C2 | complement component 2 |
| C1R | complement C1r subcomponent |
| C1S | complement component 1, s subcomponent |
| C3AR1 | complement component 3a receptor 1 |
| C5 | complement component 5 |
| C5AR1 | complement component 5a receptor 1 |
| C8A | complement component 8 alpha subunit |
| C6 | complement component 6 |
| C9 | complement component 9 |
| C7 | complement component 7 |
| C5AR2 | complement component 5a receptor 2 |

### Focal Junction Assembly

| Name | Description |
|---|---|
| ACTN1 | actinin alpha 1 |
| CAPN1 | calpain 1 |
| DAB2 | DAB2, clathrin adaptor protein |
| DOCK1 | dedicator of cytokinesis 1 |
| FN1 | fibronectin 1 |
| ILK | integrin linked kinase |
| ITGA5 | integrin subunit alpha 5 |
| ITGB1 | integrin subunit beta 1 |
| ITGAV | integrin subunit alpha V |
| ITGB3 | integrin subunit beta 3 |
| LASP1 | LIM and SH3 protein 1 |
| LIMS1 | LIM zinc finger domain containing 1 |
| PTK2 | protein tyrosine kinase 2 |
| PTPN12 | protein tyrosine phosphatase, non-receptor type 12 |
| PXN | paxillin |
| SRC | SRC proto-oncogene, non-receptor tyrosine kinase |
| TLN1 | talin 1 |
| TNS1 | tensin 1 |
| VASP | vasodilator-stimulated phosphoprotein |
| VCL | vinculin |
| VTN | vitronectin |
| ZYX | zyxin |
| ITGB1BP1 | integrin subunit beta 1 binding protein 1 |
| BCAR1 | BCAR1, Cas family scaffolding protein |
| ELMO1 | engulfment and cell motility 1 |
| DLC1 | DLC1 Rho GTPase activating protein |
| FERMT2 | fermitin family member 2 |
| PIP5K1C | phosphatidylinositol-4-phosphate 5-kinase type 1 gamma |
| FBLIM1 | filamin binding LIM protein 1 |
| PARVA | parvin alpha |

### Glycolysis

| Name | Description |
|---|---|
| ALDOA | aldolase, fructose-bisphosphate A |
| ALDOB | aldolase, fructose-bisphosphate B |
| ENO1 | enolase 1 |
| ENO3 | enolase 3 (beta, muscle) |
| ENO2 | enolase 2 (gamma, neuronal) |
| GAPDH | glyceraldehyde-3-phosphate dehydrogenase |
| GCK | glucokinase |
| GPI | glucose-6-phosphate isomerase |
| HK2 | hexokinase 2 |
| HK3 | hexokinase 3 |
| HK1 | hexokinase 1 |
| PGAM2 | phosphoglycerate mutase 2 |
| PFKM | phosphofructokinase, muscle |
| PGAM1 | phosphoglycerate mutase 1 |
| PGK1 | phosphoglycerate kinase 1 |
| PKM | pyruvate kinase, muscle |
| PKLR | pyruvate kinase, liver and RBC |
| TPI1 | triosephosphate isomerase 1 |

### Histidine-Rich Glycoprotein (HRG)

| Name | Description |
|---|---|
| SERPINC1 | serpin family C member 1 |
| CASP3 | caspase 3 |
| CD36 | CD36 molecule |
| F12 | coagulation factor XII |
| FGF2 | fibroblast growth factor 2 |
| SERPIND1 | serpin family D member 1 |
| HRG | histidine rich glycoprotein |
| IFNG | interferon, gamma |
| IL2 | interleukin 2 |
| SERPINA5 | serpin family A member 5 |
| PLG | plasminogen |
| THBS1 | thrombospondin 1 |
| THBS2 | thrombospondin 2 |
| HPSE | heparanase |
| ADAMTSL1 | ADAMTS like 1 |

### Lipogenesis Regulation in Adipocytes

| Name | Description |
|---|---|
| ACACA | acetyl-CoA carboxylase alpha |
| ACACB | acetyl-CoA carboxylase beta |
| ACLY | ATP citrate lyase |
| AKT1 | v-akt murine thymoma viral oncogene homolog 1 |
| CD36 | CD36 molecule |
| ACSL1 | acyl-CoA synthetase long-chain family member 1 |
| FASN | fatty acid synthase |
| GSK3B | glycogen synthase kinase 3 beta |
| INS | insulin |
| INSR | insulin receptor |
| IRS1 | insulin receptor substrate 1 |
| LPL | lipoprotein lipase |
| PDPK1 | 3-phosphoinositide dependent protein kinase 1 |
| PPARA | peroxisome proliferator activated receptor alpha |
| SCD | stearoyl-CoA desaturase (delta-9-desaturase) |
| SLC2A1 | solute carrier family 2 member 1 |
| SLC2A4 | solute carrier family 2 member 4 |
| SREBF1 | sterol regulatory element binding transcription factor 1 |
| IRS2 | insulin receptor substrate 2 |
| DGAT1 | diacylglycerol O-acyltransferase 1 |
| AGPAT2 | 1-acylglycerol-3-phosphate O-acyltransferase 2 |
| LPIN1 | lipin 1 |
| DGAT2 | diacylglycerol O-acyltransferase 2 |
| GPAT3 | glycerol-3-phosphate acyltransferase 3 |
| SLC27A1 | solute carrier family 27 member 1 |

### Microtubule Cytoskeleton

| Name | Description |
|---|---|
| DIAPH1 | diaphanous related formin 1 |
| DPYSL2 | dihydropyrimidinase like 2 |
| STMN1 | stathmin 1 |
| MAP2 | microtubule associated protein 2 |
| MAP4 | microtubule associated protein 4 |
| MAPT | microtubule associated protein tau |
| CLIP1 | CAP-Gly domain containing linker |
| | protein 1 |
| AURKB | aurora kinase B |
| KIF14 | kinesin family member 14 |
| KIF2C | kinesin family member 2C |
| TPPP | tubulin polymerization promoting |
| | protein |
| CLASP1 | cytoplasmic linker associated protein 1 |

### Neutrophil Activation via Adherence on Endothelial Cells

| Name | Description |
|---|---|
| ACTN1 | actinin alpha 1 |
| BTK | Bruton tyrosine kinase |
| CD34 | CD34 molecule |
| CDC42 | cell division cycle 42 |
| CR1 | complement component 3b/4b receptor 1 |
| | (Knops blood group) |
| CYBA | cytochrome b-245 alpha chain |
| CYBB | cytochrome b-245, beta polypeptide |
| FCGR2A | Fc fragment of IgG receptor IIa |
| FPR1 | formyl peptide receptor 1 |
| GNAI1 | G protein subunit alpha i1 |
| ICAM1 | intercellular adhesion molecule 1 |
| ICAM2 | intercellular adhesion molecule 2 |
| CXCL8 | C—X—C motif chemokine ligand 8 |
| CXCR2 | C—X—C motif chemokine receptor 2 |
| ITGB1 | integrin subunit beta 1 |
| ITGAM | integrin subunit alpha M |
| ITGB2 | integrin subunit beta 2 |
| ITGA9 | integrin subunit alpha 9 |
| ITGAL | integrin subunit alpha L |
| ITGB3 | integrin subunit beta 3 |
| ITPR1 | inositol 1,4,5-trisphosphate receptor type 1 |
| MME | membrane metallo-endopeptidase |
| MMP9 | matrix metallopeptidase 9 |
| NCF4 | neutrophil cytosolic factor 4 |
| NCF2 | neutrophil cytosolic factor 2 |
| NFKBIA | NFKB inhibitor alpha |
| PDPK1 | 3-phosphoinositide dependent protein |
| | kinase 1 |
| PLCG1 | phospholipase C gamma 1 |
| PRKCA | protein kinase C alpha |
| PRKCB | protein kinase C beta |
| PRKCZ | protein kinase C zeta |
| PRKCD | protein kinase C delta |
| MAPK3 | mitogen-activated protein kinase 3 |
| MAPK1 | mitogen-activated protein kinase 1 |
| MAP2K1 | mitogen-activated protein kinase kinase 1 |
| MAP2K2 | mitogen-activated protein kinase kinase 2 |
| PTK2 | protein tyrosine kinase 2 |
| PXN | paxillin |
| RAC1 | ras-related C3 botulinum toxin substrate 1 |
| | (rho family, small GTP binding protein |
| | Rac1) |
| RAF1 | Raf-1 proto-oncogene, serine/threonine |
| | kinase |
| SELE | selectin E |
| SELL | selectin L |
| SELPLG | selectin P ligand |
| SELP | selectin P |
| SYK | spleen tyrosine kinase |
| TLN1 | talin 1 |
| TNS1 | tensin 1 |
| VCAM1 | vascular cell adhesion molecule 1 |
| VCL | vinculin |
| VAV1 | vav guanine nucleotide exchange factor 1 |
| MADCAM1 | mucosal vascular addressin cell adhesion |
| | molecule 1 |
| IQGAP1 | IQ motif containing GTPase activating |
| | protein 1 |
| BCAR1 | BCAR1, Cas family scaffolding protein |
| WASF2 | WAS protein family member 2 |
| BAIAP2 | BAI1 associated protein 2 |
| PLCB1 | phospholipase C beta 1 |
| NCF1 | neutrophil cytosolic factor 1 |

### Plasmin Effects in Inflammation

| Name | Description |
|---|---|
| A2M | alpha-2-macroglobulin |
| AKT1 | v-akt murine thymoma viral oncogene |
| | homolog 1 |
| ANXA2 | annexin A2 |
| RHOA | ras homolog family member A |
| SERPING1 | serpin family G member 1 |
| C1R | complement C1r subcomponent |
| C1S | complement component 1, s |
| | subcomponent |
| CD40 | CD40 molecule |
| CDC42 | cell division cycle 42 |
| ATF2 | activating transcription factor 2 |
| ENO1 | enolase 1 |
| F2R | coagulation factor II thrombin receptor |
| F12 | coagulation factor XII |
| FN1 | fibronectin 1 |
| FOS | FBJ murine osteosarcoma viral |
| | oncogene homolog |
| GNA15 | G protein subunit alpha 15 |
| GNA12 | G protein subunit alpha 12 |
| GNAQ | G protein subunit alpha q |
| IL1B | interleukin 1 beta |
| IKBKB | inhibitor of kappa light polypeptide gene |
| | enhancer in B-cells, kinase beta |
| IL1A | interleukin 1 alpha |
| JAK1 | Janus kinase 1 |
| JUN | jun proto-oncogene |
| KLK1 | kallikrein 1 |
| KLKB1 | kallikrein B1 |
| KNG1 | kininogen 1 |
| MAP3K1 | mitogen-activated protein kinase kinase |
| | kinase 1 |
| MAP3K11 | mitogen-activated protein kinase kinase |
| | kinase 11 |
| MMP3 | matrix metallopeptidase 3 |
| MMP13 | matrix metallopeptidase 13 |
| MMP1 | matrix metallopeptidase 1 |
| NFKBIA | NFKB inhibitor alpha |
| SERPINE1 | serpin family E member 1 |
| PDPK1 | 3-phosphoinositide dependent protein |
| | kinase 1 |
| PLAT | plasminogen activator, tissue type |
| PLAU | plasminogen activator, urokinase |
| PLAUR | plasminogen activator, urokinase |
| | receptor |
| PLG | plasminogen |
| SERPINF2 | serpin family F member 2 |
| PRKCE | protein kinase C epsilon |
| MAPK3 | mitogen-activated protein kinase 3 |
| MAPK1 | mitogen-activated protein kinase 1 |
| MAP2K3 | mitogen-activated protein kinase kinase 3 |
| MAP2K1 | mitogen-activated protein kinase kinase 1 |
| MAP2K7 | mitogen-activated protein kinase kinase 7 |
| MAP2K6 | mitogen-activated protein kinase kinase 6 |
| MAP2K2 | mitogen-activated protein kinase kinase 2 |
| PXN | paxillin |
| RAC1 | ras-related C3 botulinum toxin substrate |
| | 1 (rho family, small GTP binding protein |
| | Rac1) |
| RAF1 | Raf-1 proto-oncogene, serine/threonine |
| | kinase |
| RASGRF1 | Ras protein specific guanine nucleotide |
| | releasing factor 1 |
| S100A10 | S100 calcium binding protein A10 |
| CCL2 | C-C motif chemokine ligand 2 |
| CCL20 | C-C motif chemokine ligand 20 |
| MAP2K4 | mitogen-activated protein kinase kinase 4 |
| SRC | SRC proto-oncogene, non-receptor |
| | tyrosine kinase |
| STAT1 | signal transducer and activator of |
| | transcription 1 |
| STAT3 | signal transducer and activator of |
| | transcription 3 |
| TF | transferrin |
| TGFB1 | transforming growth factor beta 1 |
| TNF | tumor necrosis factor |
| TYK2 | tyrosine kinase 2 |
| TFPI2 | tissue factor pathway inhibitor 2 |
| BCAR1 | BCAR1, Cas family scaffolding protein |
| RASGRP1 | RAS guanyl releasing protein 1 |
| GNA13 | G protein subunit alpha 13 |
| SPINK5 | serine peptidase inhibitor, Kazal type 5 |
| PLGRKT | plasminogen receptor with a C-terminal |
| | lysine |

### Platelet Activation via Adhesion Molecules

| Name | Description |
|---|---|
| AKT1 | v-akt murine thymoma viral oncogene |
| | homolog 1 |
| RHOA | ras homolog family member A |
| BRAF | B-Raf proto-oncogene, serine/threonine |
| | kinase |
| CD40LG | CD40 ligand |
| CDC42 | cell division cycle 42 |
| MAPK14 | mitogen-activated protein kinase 14 |
| HBEGF | heparin binding EGF like growth factor |
| F3 | coagulation factor III, tissue factor |
| PTK2B | protein tyrosine kinase 2 beta |
| FCER1G | Fc fragment of IgE receptor Ig |
| FYN | FYN proto-oncogene, Src family tyrosine |
| | kinase |
| GP1BB | glycoprotein Ib platelet beta subunit |
| GP5 | glycoprotein V platelet |
| GP1BA | glycoprotein Ib platelet alpha subunit |
| GP9 | glycoprotein IX platelet |
| GRB2 | growth factor receptor bound protein 2 |
| ARHGAP35 | Rho GTPase activating protein 35 |
| CXCL8 | C—X—C motif chemokine ligand 8 |
| IL6 | interleukin 6 |
| ITGA6 | integrin subunit alpha 6 |
| ITGA2 | integrin subunit alpha 2 |
| ITGA2B | integrin subunit alpha 2b |
| ITGB1 | integrin subunit beta 1 |
| ITGB3 | integrin subunit beta 3 |
| ITPR1 | inositol 1,4,5-trisphosphate receptor type 1 |
| LCP2 | lymphocyte cytosolic protein 2 |
| LYN | LYN proto-oncogene, Src family tyrosine |
| | kinase |
| MAP3K1 | mitogen-activated protein kinase kinase |
| | kinase 1 |
| MYLK | myosin light chain kinase |
| NOS3 | nitric oxide synthase 3 |
| PDPK1 | 3-phosphoinositide dependent protein |
| | kinase 1 |
| PLCG1 | phospholipase C gamma 1 |
| PLCG2 | phospholipase C gamma 2 |
| PRKCA | protein kinase C alpha |
| MAPK3 | mitogen-activated protein kinase 3 |
| MAPK1 | mitogen-activated protein kinase 1 |
| PRKG1 | protein kinase, cGMP-dependent, type I |
| MAP2K1 | mitogen-activated protein kinase kinase 1 |
| MAP2K6 | mitogen-activated protein kinase kinase 6 |
| MAP2K2 | mitogen-activated protein kinase kinase 2 |
| PTK2 | protein tyrosine kinase 2 |
| PTPRJ | protein tyrosine phosphatase, receptor type J |
| PXN | paxillin |
| RAC1 | ras-related C3 botulinum toxin substrate 1 |
| | (rho family, small GTP binding protein |
| | Rac1) |
| RAF1 | Raf-1 proto-oncogene, serine/threonine |
| | kinase |
| RAP1A | RAP1A, member of RAS oncogene family |
| ROCK1 | Rho associated coiled-coil containing |
| | protein kinase 1 |
| SOS1 | SOS Ras/Rac guanine nucleotide exchange |
| | factor 1 |
| SRC | SRC proto-oncogene, non-receptor tyrosine |
| | kinase |
| STX4 | syntaxin 4 |
| SYK | spleen tyrosine kinase |
| TLN1 | talin 1 |
| TNF | tumor necrosis factor |
| TNS1 | tensin 1 |
| VCL | vinculin |
| VAV1 | vav guanine nucleotide exchange factor 1 |
| WAS | Wiskott-Aldrich syndrome |
| VWF | von Willebrand factor |
| VAMP8 | vesicle associated membrane protein 8 |
| SNAP23 | synaptosome associated protein 23 kDa |
| IQGAP1 | IQ motif containing GTPase activating |
| | protein 1 |
| BCAR1 | BCAR1, Cas family scaffolding protein |
| PIP5K1C | phosphatidylinositol-4-phosphate 5-kinase |
| | type 1 gamma |
| LAT | linker for activation of T-cells |
| GP6 | glycoprotein VI platelet |
| APBB1IP | amyloid beta precursor protein binding |
| | family B member 1 interacting protein |

### Platelet Activation via GPCR Signaling

| Name | Description |
|---|---|
| ADCY3 | adenylate cyclase 3 |
| ADORA2A | adenosine A2a receptor |
| AKT1 | v-akt murine thymoma viral oncogene |
| | homolog 1 |
| RHOA | ras homolog family member A |
| BRAF | B-Raf proto-oncogene, serine/threonine |
| | kinase |
| CDC42 | cell division cycle 42 |
| F2 | coagulation factor II, thrombin |
| F2R | coagulation factor II thrombin receptor |
| GNAS | GNAS complex locus |
| GNAQ | G protein subunit alpha q |
| HTR2A | 5-hydroxytryptamine receptor 2A |
| ITGA2 | integrin subunit alpha 2 |
| ITGB1 | integrin subunit beta 1 |
| ITGB3 | integrin subunit beta 3 |
| ITPR1 | inositol 1,4,5-trisphosphate receptor type 1 |
| MPL | MPL proto-oncogene, thrombopoietin |
| | receptor |
| MYLK | myosin light chain kinase |
| NOS3 | nitric oxide synthase 3 |
| P2RY1 | purinergic receptor P2Y1 |
| PDPK1 | 3-phosphoinositide dependent protein |
| | kinase 1 |
| PRKCA | protein kinase C alpha |
| MAPK3 | mitogen-activated protein kinase 3 |
| MAPK1 | mitogen-activated protein kinase 1 |
| PRKG1 | protein kinase, cGMP-dependent, type I |
| MAP2K1 | mitogen-activated protein kinase kinase 1 |
| MAP2K2 | mitogen-activated protein kinase kinase 2 |
| PTAFR | platelet activating factor receptor |
| PTGER4 | prostaglandin E receptor 4 |
| PTGIR | prostaglandin I2 (prostacyclin) receptor (IP) |
| PTGFR | prostaglandin F receptor |
| RAC1 | ras-related C3 botulinum toxin substrate 1 |
| | (rho family, small GTP binding protein |
| | Rac1) |
| RAF1 | Raf-1 proto-oncogene, serine/threonine |
| | kinase |
| RAP1A | RAP1A, member of RAS oncogene family |
| RASGRF1 | Ras protein specific guanine nucleotide |
| | releasing factor 1 |
| ROCK1 | Rho associated coiled-coil containing |
| | protein kinase 1 |
| STX4 | syntaxin 4 |
| TBXA2R | thromboxane A2 receptor |
| THPO | thrombopoietin |
| TLN1 | talin 1 |
| WAS | Wiskott-Aldrich syndrome |
| VAMP8 | vesicle associated membrane protein 8 |
| SNAP23 | synaptosome associated protein 23 kDa |
| IQGAP1 | IQ motif containing GTPase activating |
| | protein 1 |
| F2RL3 | F2R like thrombin/trypsin receptor 3 |
| RASGRP1 | RAS guanyl releasing protein 1 |
| GNA13 | G protein subunit alpha 13 |
| PLCB1 | phospholipase C beta 1 |
| APBB1IP | amyloid beta precursor protein binding |
| | family B member 1 interacting protein |
| P2RY12 | purinergic receptor P2Y12 |

### Positive Acute Phase Proteins Synthesis

| Name | Description |
|---|---|
| A2M | alpha-2-macroglobulin |
| SERPINA3 | serpin family A member 3 |
| C3 | complement component 3 |
| CEBPB | CCAAT/enhancer binding protein beta |
| CEBPD | CCAAT/enhancer binding protein delta |
| CP | ceruloplasmin (ferroxidase) |
| CRP | C-reactive protein, pentraxin-related |
| EGR1 | early growth response 1 |
| F8 | coagulation factor VIII |
| FN1 | fibronectin 1 |
| GHR | growth hormone receptor |
| GH1 | growth hormone 1 |
| GRB2 | growth factor receptor bound protein 2 |
| HIF1A | hypoxia inducible factor 1 alpha subunit |
| HNF4A | hepatocyte nuclear factor 4 alpha |
| HP | haptoglobin |
| HPX | hemopexin |
| IL1B | interleukin 1 beta |
| IL1RAP | interleukin 1 receptor accessory protein |
| IL1A | interleukin 1 alpha |
| IL1R1 | interleukin 1 receptor type 1 |
| IL6ST | interleukin 6 signal transducer |
| IL6R | interleukin 6 receptor |
| IL6 | interleukin 6 |
| IRAK1 | interleukin 1 receptor associated kinase 1 |
| JAK1 | Janus kinase 1 |
| JAK2 | Janus kinase 2 |
| LBP | lipopolysaccharide binding protein |
| MBL2 | mannose binding lectin 2 |
| MYD88 | myeloid differentiation primary response 88 |
| NFKBIA | NFKB inhibitor alpha |
| ORM1 | orosomucoid 1 |
| SERPINE2 | serpin family E member 2 |
| SERPINA1 | serpin family A member 1 |
| PPARA | peroxisome proliferator activated receptor |
| | alpha |
| MAPK8 | mitogen-activated protein kinase 8 |
| MAPK1 | mitogen-activated protein kinase 1 |
| MAP2K1 | mitogen-activated protein kinase kinase 1 |
| MAP2K2 | mitogen-activated protein kinase kinase 2 |
| PTPN11 | protein tyrosine phosphatase, non-receptor |
| | type 11 |
| RAF1 | Raf-1 proto-oncogene, serine/threonine |
| | kinase |
| SAA1 | serum amyloid A1 |
| MAP2K4 | mitogen-activated protein kinase kinase 4 |
| SOS1 | SOS Ras/Rac guanine nucleotide exchange |
| | factor 1 |
| SRF | serum response factor |
| STAT3 | signal transducer and activator of |
| | transcription 3 |
| STAT5A | signal transducer and activator of |
| | transcription 5A |
| MAP3K7 | mitogen-activated protein kinase kinase |
| | kinase 7 |
| TLR4 | toll like receptor 4 |
| TNF | tumor necrosis factor |
| TNFRSF1B | tumor necrosis factor receptor superfamily |
| | member 1B |
| TNFRSF1A | tumor necrosis factor receptor superfamily |
| | member 1A |
| TRAF6 | TNF receptor associated factor 6 |
| TRAF2 | TNF receptor associated factor 2 |
| TYK2 | tyrosine kinase 2 |
| VWF | von Willebrand factor |
| TRADD | TNFRSF1A associated via death domain |
| MAPKAPK2 | mitogen-activated protein kinase-activated |
| | protein kinase 2 |
| IRAK4 | interleukin 1 receptor associated kinase 4 |

### Protein Folding

| Name | Description |
|---|---|
| BAG1 | BCL2 associated athanogene 1 |
| FKBP4 | FK506 binding protein 4 |
| HSPA1A | heat shock protein family A (Hsp70) member |
| | 1A |
| HSP90AA1 | heat shock protein 90 kDa alpha family class A |
| | member 1 |
| HSPA8 | heat shock protein family A (Hsp70) member 8 |
| ST13 | suppression of tumorigenicity 13 (colon |
| | carcinoma) (Hsp70 interacting protein) |
| HSP90B1 | heat shock protein 90 kDa beta family member 1 |
| TOMM70 | translocase of outer mitochondrial membrane |
| | 70 |
| STUB1 | STIP1 homology and U-box containing protein 1 |
| CDC37 | cell division cycle 37 |
| HSPBP1 | HSPA (heat shock 70 kDa) binding protein, |
| | cytoplasmic cochaperone 1 |
| HSPA14 | heat shock protein family A (Hsp70) member |
| | 14 |
| UNC45A | unc-45 myosin chaperone A |

### Scavenger Receptors in Platelet Activation

| Name | Description |
|---|---|
| RHOA | ras homolog family member A |
| CD9 | CD9 molecule |
| CD36 | CD36 molecule |
| SCARB1 | scavenger receptor class B member 1 |
| MAPK14 | mitogen-activated protein kinase 14 |
| FYN | FYN proto-oncogene, Src family tyrosine |
| | kinase |
| GP1BB | glycoprotein Ib platelet beta subunit |
| GP1BA | glycoprotein Ib platelet alpha subunit |
| ITPR1 | inositol 1,4,5-trisphosphate receptor type 1 |
| LCP2 | lymphocyte cytosolic protein 2 |
| LYN | LYN proto-oncogene, Src family tyrosine |
| | kinase |
| MATK | megakaryocyte-associated tyrosine kinase |
| MAP3K1 | mitogen-activated protein kinase kinase |
| | kinase 1 |
| PLCG2 | phospholipase C gamma 2 |
| PRKCA | protein kinase C alpha |
| MAPK8 | mitogen-activated protein kinase 8 |
| MAP2K6 | mitogen-activated protein kinase kinase 6 |
| ROCK1 | Rho associated coiled-coil containing protein |
| | kinase 1 |
| MAP2K4 | mitogen-activated protein kinase kinase 4 |
| SYK | spleen tyrosine kinase |
| VAV1 | vav guanine nucleotide exchange factor 1 |
| YES1 | YES proto-oncogene 1, Src family tyrosine |
| | kinase |
| LAT | linker for activation of T-cells |

### Scavenger Receptors in Platelet Aggregation

| Name | Description |
|---|---|
| RHOA | ras homolog family member A |
| CD68 | CD68 molecule |
| GNAQ | G protein subunit alpha q |
| ITPR1 | inositol 1,4,5-trisphosphate receptor type 1 |
| OLR1 | oxidized low density lipoprotein receptor 1 |
| P2RY1 | purinergic receptor P2Y1 |
| PDPK1 | 3-phosphoinositide dependent protein |
| | kinase 1 |
| RAC1 | ras-related C3 botulinum toxin substrate |
| | 1 (rho family, small GTP binding protein |
| | Rac1) |
| ROCK1 | Rho associated coiled-coil containing |
| | protein kinase 1 |
| TLN1 | talin 1 |
| IQGAP1 | IQ motif containing GTPase activating |
| | protein 1 |
| PLCB1 | phospholipase C beta 1 |
| APBB1IP | amyloid beta precursor protein binding |
| | family B member 1 interacting protein |

### TAM Receptors in Platelet Aggregation

| Name | Description |
|---|---|
| AKT1 | v-akt murine thymoma viral oncogene |
| | homolog 1 |
| AXL | AXL receptor tyrosine kinase |
| GAS6 | growth arrest specific 6 |
| PDPK1 | 3-phosphoinositide dependent protein |
| | kinase 1 |
| PROS1 | protein S (alpha) |
| TYRO3 | TYRO3 protein tyrosine kinase |
| VWF | von Willebrand factor |
| MERTK | MER proto-oncogene, tyrosine kinase |

### Vascular Endothelial Cell Activation by Blood Coagulation Factors

| Name | Description |
|---|---|
| AKT1 | v-akt murine thymoma viral oncogene |
| | homolog 1 |
| BDKRB2 | bradykinin receptor B2 |
| CDH5 | cadherin 5 |
| CTGF | connective tissue growth factor |
| HBEGF | heparin binding EGF like growth factor |
| F2 | coagulation factor II, thrombin |
| F2RL2 | coagulation factor II thrombin receptor like 2 |
| F2RL1 | F2R like trypsin receptor 1 |
| F2R | coagulation factor II thrombin receptor |
| F10 | coagulation factor X |
| F7 | coagulation factor VII |
| FOS | FBJ murine osteosarcoma viral oncogene |
| | homolog |
| GNA15 | G protein subunit alpha 15 |
| GNA11 | G protein subunit alpha 11 |
| GNAQ | G protein subunit alpha q |
| CXCL2 | C—X—C motif chemokine ligand 2 |
| ICAM1 | intercellular adhesion molecule 1 |
| CYR61 | cysteine rich angiogenic inducer 61 |
| IL1B | interleukin 1 beta |
| CXCL8 | C—X—C motif chemokine ligand 8 |
| IL6 | interleukin 6 |
| IRAK1 | interleukin 1 receptor associated kinase 1 |
| ITPR1 | inositol 1,4,5-trisphosphate receptor type 1 |
| JAK2 | Janus kinase 2 |
| JUN | jun proto-oncogene |
| KNG1 | kininogen 1 |
| MYD88 | myeloid differentiation primary response |
| | 88 |
| NFKBIA | NFKB inhibitor alpha |
| YBX1 | Y-box binding protein 1 |
| PDPK1 | 3-phosphoinositide dependent protein |
| | kinase 1 |
| PLCB3 | phospholipase C beta 3 |
| PRKCA | protein kinase C alpha |
| MAPK3 | mitogen-activated protein kinase 3 |
| MAPK1 | mitogen-activated protein kinase 1 |
| MAP2K1 | mitogen-activated protein kinase kinase 1 |
| MAP2K2 | mitogen-activated protein kinase kinase 2 |
| PROC | protein C, inactivator of coagulation |
| | factors Va and VIIIa |
| PROS1 | protein S (alpha) |
| RAF1 | Raf-1 proto-oncogene, serine/threonine |
| | kinase |
| RASGRF1 | Ras protein specific guanine nucleotide |
| | releasing factor 1 |
| CCL2 | C-C motif chemokine ligand 2 |
| SRC | SRC proto-oncogene, non-receptor |
| | tyrosine kinase |
| STAT1 | signal transducer and activator of |
| | transcription 1 |
| STAT3 | signal transducer and activator of |
| | transcription 3 |
| TFPI | tissue factor pathway inhibitor |
| TLR4 | toll like receptor 4 |
| TNF | tumor necrosis factor |
| TRAF6 | TNF receptor associated factor 6 |
| TYK2 | tyrosine kinase 2 |
| VCAM1 | vascular cell adhesion molecule 1 |
| VEGFA | vascular endothelial growth factor A |
| F2RL3 | F2R like thrombin/trypsin receptor 3 |
| RASGRP1 | RAS guanyl releasing protein 1 |
| PROCR | protein C receptor |
| PLCB1 | phospholipase C beta 1 |
| IRAK4 | interleukin 1 receptor associated kinase 4 |
| OCLN | occludin |

1. A method for screening, monitoring and/or treating a gastrointestinal (GI) cancer patient, the method comprising:
obtaining a sample from the patient,
isolating glycosylated proteins from the sample,
analyzing the isolated glycosylated proteins for biomarkers listed in Tables 1A, 2A, 3A, 4A, 5A, 6A, 7A, or any combination thereof, and/or
grouping the isolated glycosylated proteins into a profile of pathways, and
matching the obtained profile at least partially with at least one profile selected from the group of profiles of Tables 1, 2, 3, 4, 5, 6, 7, 8, and any combination thereof.

2. The method of claim 1, wherein the GI cancer is selected from the group consisting of colorectal cancer, gastric cancer, liver cancer, and pancreatic cancer.

3. The method of claim 1, wherein the sample is selected from the group consisting of a human tissue biopsy or biosample including pancreas biopsy sample, gastrointestinal sample, blood sample, plasma sample, serum sample, circulating tumor cells sample, tear sample, saliva sample, sperm sample, urine sample, fecal sample and hair sample.

4. The method of claim 1, wherein the sample is analyzed using one or more techniques selected from the group consisting of chromatography, gas chromatography, liquid chromatography, mass spectrometry, ELISA, antibody linkage, immunoassay, biochip assay, microarray, nanoassay, spectroscopy, a multiplex molecular assay or techniques which utilize a fluorescent, enzyme, radioactive, metallic, biotin, chemiluminescent, bioluminescent molecule assay.

5. The method of claim 1, wherein the sample is analyzed using a combination of a detection techniques of nucleic acids and proteins or peptides.

6. The method of claim 1, wherein biomarkers of Tables 1, 2, 3, 4, 5, 6, 7, 1A, 2A, 3A, 4A, 5A, 6A and/or 7A are immobilized on a solid support.

7. The method of claim 1, wherein the analyzing and/or matching is conducted by reacting the patient's sample with at least one antibody and/or a synthetic compound, probe or protein-chemistry based reagent specific to at least one biomarker and/or glycobiomarker of Tables 1, 2, 3, 4, 5, 6, 7, 1A, 2A, 3A, 4A, 5A, 6A and/or 7A.

8. (canceled)

9. (canceled)

10. (canceled)

11. A method for detecting or monitoring a disorder of the pancreas, the method comprising obtaining a sample from a patient and testing the sample for at least one or more biomarker and/or glycobiomarker selected from Tables 5A, 5, 6A, 6 and 9.

12. The method of claim 11, wherein the disorder of the pancreas is selected from the group consisting of acute pancreatitis, chronic pancreatitis, hereditary pancreatitis, pancreatic neoplasm, and pancreatic cancer.

13. The method of claim 11, wherein the sample is selected from the group consisting of a human tissue biopsy or biosample including pancreas biopsy sample, gastrointestinal sample, blood sample, plasma sample, serum sample, circulating tumor cells sample, tear sample, saliva sample, sperm sample, urine sample, fecal sample and hair sample.

14. The method of claim 11, wherein the sample is analyzed using one or more techniques selected from the group consisting of chromatography, gas chromatography, liquid chromatography, mass spectrometry, ELISA, antibody linkage, immunoassay, biochip assay, microarray, nanoassay, spectroscopy, a multiplex molecular assay or techniques which utilize a fluorescent, enzyme, radioactive, metallic, biotin, chemiluminescent, bioluminescent molecule assay.

15. The method of claim 11, wherein the sample is analyzed using a combination of a detection techniques of nucleic acids and proteins or peptides.

16. (canceled)

17. The method of claim 11, wherein the testing is conducted by reacting the patient's sample with at least one antibody or protein chemistry-based reagent specific to at least one biomarker and/or glycobiomarker of Tables 5A, 5, 6A, 6 and 9.

18. The method of claim 11, wherein the testing is conducted by reacting the patient's sample with at least one protein specific to at least one biomarker and/or glycobiomarker of Tables 5A, 5, 6A, 6 and 9.

19. The method of claim 11, wherein the testing is conducted by reacting the patient's sample with a synthetic compound or probe which react with at least one protein specific to at least one biomarker and/or glycobiomarker of Tables 5A, 5, 6A, 6 and 9.

20. A method for treating a disorder of the pancreas, the method comprising obtaining a sample from a mammal in need of the treatment and testing the sample for at least one or more biomarker and/or glycobiomarker selected from Tables 5A, 5, 6A, 6 and 9.

21.-28. (canceled)
